SEQUENCE LISTING (17 sequences)

SEQ ID NO: 1 - skipped

SEQ ID NO: 2 - DNA, 19 bp, synthetic construct
  misc_feature 1..19: note = Description of Artificial Sequence: Synthetic oligonucleotide
  source 1..19: mol_type = other DNA
acggaccggaaatccggtt

SEQ ID NO: 3 - DNA, 18 bp, synthetic construct
  misc_feature 1..18: note = Description of Artificial Sequence: Synthetic oligonucleotide
  source 1..18: mol_type = other DNA
tacaaagatcaaagggtt

SEQ ID NO: 4 - DNA, 31 bp, synthetic construct
  misc_feature 1..31: note = Description of Artificial Sequence: Synthetic oligonucleotide
  source 1..31: mol_type = other DNA
tggggactttccagtttctggaaagtcccca

SEQ ID NO: 5 - DNA, 14 bp, synthetic construct
  misc_feature 1..14: note = Description of Artificial Sequence: Synthetic oligonucleotide
  source 1..14: mol_type = other DNA
ctagatttcccgcg

SEQ ID NO: 6 - DNA, 14 bp, synthetic construct
  misc_feature 1..14: note = Description of Artificial Sequence: Synthetic oligonucleotide
  source 1..14: mol_type = other DNA
ctagcgcgggaaat

SEQ ID NO: 7 - DNA, 13 bp, synthetic construct
  misc_feature 1..13: note = Description of Artificial Sequence: Synthetic oligonucleotide
  source 1..13: mol_type = other DNA
ctagcgcggaaat

SEQ ID NO: 8 - DNA, 19 bp, unidentified
  misc_feature 1..19: note = Description of Unknown: TNF alpha sequence
  source 1..19: mol_type = unassigned DNA
aacagggggctttccctcc

SEQ ID NO: 9 - DNA, 19 bp, Human immunodeficiency virus 1
  source 1..19: mol_type = other DNA
cgctggggactttccacgg

SEQ ID NO: 10 - DNA, 31 bp, synthetic construct; identical to SEQ ID NO: 4
  misc_feature 1..31: note = Description of Artificial Sequence: Synthetic oligonucleotide
  source 1..31: mol_type = other DNA

SEQ ID NO: 11 - DNA, 31 bp, synthetic construct; identical to SEQ ID NO: 4
  misc_feature 1..31: note = Description of Artificial Sequence: Synthetic oligonucleotide
  source 1..31: mol_type = other DNA

SEQ ID NO: 12 - DNA, 31 bp, synthetic construct; identical to SEQ ID NO: 4
  misc_feature 1..31: note = Description of Artificial Sequence: Synthetic oligonucleotide
  source 1..31: mol_type = other DNA

SEQ ID NO: 13 - DNA, 14 bp, synthetic construct
  misc_feature 1..14: note = Description of Artificial Sequence: Synthetic oligonucleotide
  source 1..14: mol_type = other DNA
ctagatttcccgcg

SEQ ID NO: 14 - DNA, 14 bp, synthetic construct
  misc_feature 1..14: note = Description of Artificial Sequence: Synthetic oligonucleotide
  source 1..14: mol_type = other DNA
ctagatttcccgcg

SEQ ID NO: 15 - DNA, 31 bp, synthetic construct; identical to SEQ ID NO: 4
  misc_feature 1..31: note = Description of Artificial Sequence: Synthetic oligonucleotide
  source 1..31: mol_type = other DNA

SEQ ID NO: 16 - DNA, 31 bp, synthetic construct; identical to SEQ ID NO: 4
  misc_feature 1..31: note = Description of Artificial Sequence: Synthetic oligonucleotide
  source 1..31: mol_type = other DNA

SEQ ID NO: 17 - DNA, 14 bp, synthetic construct
  misc_feature 1..14: note = Description of Artificial Sequence: Synthetic oligonucleotide
  source 1..14: mol_type = other DNA
ctagatttcccgcg

(19) United States
(12) Patent Application Publication
(10) Pub. No.: US 2024/0131173 A1
(43) Pub. Date: Apr. 25, 2024

(54) PROTACS WITH TRANSCRIPTION FACTOR TARGETING MOIETIES

(71) Applicants: Beth Israel Deaconess Medical Center, Inc., Boston, MA (US); Icahn School of Medicine at Mount Sinai, New York, NY (US)

(72) Inventors: Wenyi WEI, Boston, MA (US); Jian JIN, New York, NY (US); Jing LIU, Boston, MA (US); He CHEN, New York, NY (US); Husnu Ümit KANISKAN, New York, NY (US)

(73) Assignees: Beth Israel Deaconess Medical Center, Inc., Boston, MA (US); Icahn School of Medicine at Mount Sinai, New York, NY (US)

(21) Appl. No.: 18/520,481

(22) Filed: Nov. 27, 2023

Related U.S. Application Data
(63) Continuation of application No. PCT/US2022/031317, filed on May 27, 2022.
(60) Provisional application No. 63/194,917, filed on May 28, 2021.
(60) Provisional application No. 63/245,506, filed on Sep. 17, 2021.

(51) Int. Cl. A61K 47/64; A61K 47/54; A61K 47/55

(52) U.S. Cl. CPC A61K 47/64; A61K 47/545; A61K 47/55

(57) ABSTRACT

Transcription factors (TFs) represent a major class of therapeutic targets for the treatment of human diseases including cancer. Although the biological function and even crystal structure of many TFs have been clearly elucidated, there is still no viable approach to target the majority of TFs, thus rendering them undruggable for decades. PROTACs (PROteolysis TArgeting Chimeras) have emerged as a powerful tool for the pharmaceutical development since the effect of PROTACs largely relies on engineered protein-protein interaction to aid the degradation of targets by the ubiquitin-proteasome system (UPS). The present disclosure provides a DNA-PROTAC platform for targeted degraders of individual TFs of interest. These DNA based Transcription Factor targetting PROTACS (or “TF-PROTACS”) may provide specificity to TF degradation based on the conserved DNA-binding motifs of respective TFs. We have synthesized two series of VHL-based TF-PROTACs lead compounds and measured their degradation of proteins in transcription factors: NF-κB-PROTAC (or dNF-κB) and E2F-PROTAC (or dE2F). NF-κB-PROTAC efficiently degrades p65 protein subunit in the NF-κB transcription factors in cells. E2F-PROTAC efficiently degrades E2F1 protein subunit in the E2F transcription factor in cells. Taken together, this design provides a generalizable platform of TF-PROTACs to achieve selective degradation of TFs in cells.

### CROSS-REFERENCE TO RELATED APPLICATIONS

This application is a continuation under 35 U.S.C. § 111(a) of PCT International Patent Application No. PCT/US2022/031317, filed May 27, 2022, designating the United States and published in English, which claims priority to and the benefit of U.S. Provisional Application No. 63/194,917, filed May 28, 2021, and U.S. Provisional Application No. 63/245,506, filed Sep. 17, 2021, the entire contents of each of which are incorporated by reference herein.

### STATEMENT OF RIGHTS TO INVENTIONS MADE UNDER FEDERALLY SPONSORED RESEARCH

This invention was made with government support under Grant No. CA253027 awarded by the National Institutes of Health. The government has certain rights in this invention.

### SEQUENCE LISTING

The present application contains a Sequence Listing which has been submitted electronically in XML format following conversion from the originally filed TXT format.

The entire contents of the electronic XML Sequence Listing, (Date of creation: Nov. 22, 2023; Size: 21,545 bytes; Name: 167688-020503US-Sequence_Listing.xml), is incorporated by reference herein.

### BACKGROUND OF THE INVENTION

Genetic regulation relies largely on the sequence-specific transcription factors (TFs) that recognize short DNA segments, also known as TF binding motifs, which typically locate in the enhancer and promotor regions of respective genes. These TFs promote or repress genetic transcription and play pivotal roles in the development of various human diseases, including cancer. The cancer dependency map project (DepMap) uncovered that TFs represent a major class of essential genes that maintain the proliferation and tumorigenesis of cancer cells, indicating a high potential of TFs as therapeutic targets. To date, ample attempts have attributed to develop TFs-targeting therapy using small molecular inhibitors (SMI), including NF-kB, STAT3/5, and MYC, as well as the well-defined AR and ER inhibitors. However, the majority of TFs are still lack of available SMI for possible therapeutic intervention.

In the human genome, there are approximately 1600 putative TFs, which could be classified into tens of families largely based on their distinct DNA binding domains, such as C2H2, bZIP, bHLH and Homeodomain 21. Several low-throughput and high-throughput methods including protein binding microarray (PBM), SELEX, DAP-seq, HITS-FLIP, EMSA and CHIP, have been used to define the specific DNA binding motif for each individual TF as described in Gerstein, M. B. et al. Nature 489 (2012): 91-100; Berger M. F. et al Nat Biotechnol 24.11 (2006): 1429-35; Weirauch, M. T. et al Cell 158.6 (2014): 1431-1443, and Jolma, A. et al ('ell 152.1-2 (2013): 327-39, each of which are hereby incorporated by reference in their entirety and particularly in reference to the TF binding motifs described therein. To date, the DNA binding motif have been successfully defined for more than 600 putative human TFs by experimental approaches and more than 1300 by theoretical methods, which presents valueless treasury that may aid the development of TFs-targeting therapies.

TFs typically lack active sites or allosteric regulatory pockets that normally exist in kinases or other types of enzymes, thereby making it difficult to design and screen for small molecule inhibitors (SMIs) of TFs. Given the key role of TFs in binding with specific DNA sequences to regulate genetic transcription, the DNA binding motif in theory may define the biological and potentially biochemical specificity of different TFs. Indeed, a large amount of studies have theoretically and/or experimentally defined the unique DNA binding motif for most of TFs as described in Pujato, M et al., Nucleic Acids Res 42 (2014): 13500-12, which is hereby incorporated by reference in its entirety and particularly in reference to the TF2DNA algorithm and TF binding motifs described therein. Nevertheless, identification of compounds capable of binding to TFs to modify their transcriptional activity has proved problematic such that TFs are often considered “undruggable.”

### SUMMARY

It is an object of the present disclosure to provide compounds capable of binding to transcription factors and promoting their degradation. As shown herein, by hijacking endogenous E3 ubiquitin ligase and the ubiquitin-proteasome system (UPS), PROteolysis

TArgeting Chimera (PROTAC) may incorporate DNA sequences into their protein binding moiety allowing the PROTAC to bind to TFs following endocytosis and thereby afford targeted degradation of transcription factors.

A typical PROTAC molecule consists three functional parts, a warhead to recruit and bind the protein of interest (POI), a ligand to recruit the E3 ligase, and a linker between these two moieties. The compounds of the present disclosure target TFs via the incorporation of a DNA oligomer into the PROTAC as one of the chimera.

These PROTACs may be compounds having the structure of formula (I):

ODN—L₁—ULB (I)

wherein ULB is a ubiquitin ligase binding moiety;
L₁ is absent or a linker;
ODN is a DNA oligomer protein comprising a transcription factor binding motif that binds to a transcription factor or subunit thereof (e.g., a DNA enhancer or promoter sequence);
or pharmaceutically acceptable salts of the foregoing. Typically, conjugation occurs between the DNA oligomer protein and the ULB moiety via an azide/alkynyl cycloaddition reaction to form the functioning PROTAC. Accordingly, L₁ often comprises an optionally substituted heteroarylene group (e.g., a five membered heteroarylene, a six membered heteroarylene, a divalent triazole, a divalent imidazole, a divalent pyrrole, a multicyclic (e.g., bicyclic, tricyclic) heteroarylene such as a five or six membered membered heteroaryl including triazolyl, imidazolyl, pyrrolyl which may be optionally fused to an mono or bicyclic cycloalkyl such as C₃-C₁₂ monocyclic cycloalkyl or C₄-C₁₂ bicyclic cycloalkyl including bicyclo[6.1.0] nonane). In certain aspects, L₁ comprises a triazole fused to a C₆-C₁₂ monocyclic or bicyclic cylcoalkyl (e.g., azide/alkynyl SPAAC reaction products). In some embodiments, L₁ comprises an optionally substituted mono or multicyclic heteroarylene group or an optionally substituted mono or multicyclic heterocyclene group (e.g., a five membered heterocyclene, a six membered heterocycene, a mono or multicyclic heteroarylene (e.g., dihydroisoxazolyl which may be optionally fused to a mono or bicyclic cycloalkyl such as C₃-C₁₂ monocyclic cycloalkyl or C₄-C₁₂ bicyclic cycloalkyl including bicyclo[6.1.0] nonane).

In some embodiments, the ULB binds to an E3 ubiquitin ligase. For example, the E3 ubiquitin ligase may be selected from the group consisting of von Hippel Lindau (VHL) E3 ubiquitin ligase, β-Transducin Repeat Containing (β-TRCP) E3 Ubiquitin Protein Ligase, Mouse Double Minute 2 (Mdm2) E3 Ubiquitin Protein Ligase, and a Cereblon (CRBN) E3 Ubiquitin ligase. For example, in some embodiments, the compound may conjugated through any number of conjugation opints in the E3 ubiquitin ligase. For example, the compound may have the structure of Formula (IIA), (IIB), (IIC), or (IID):

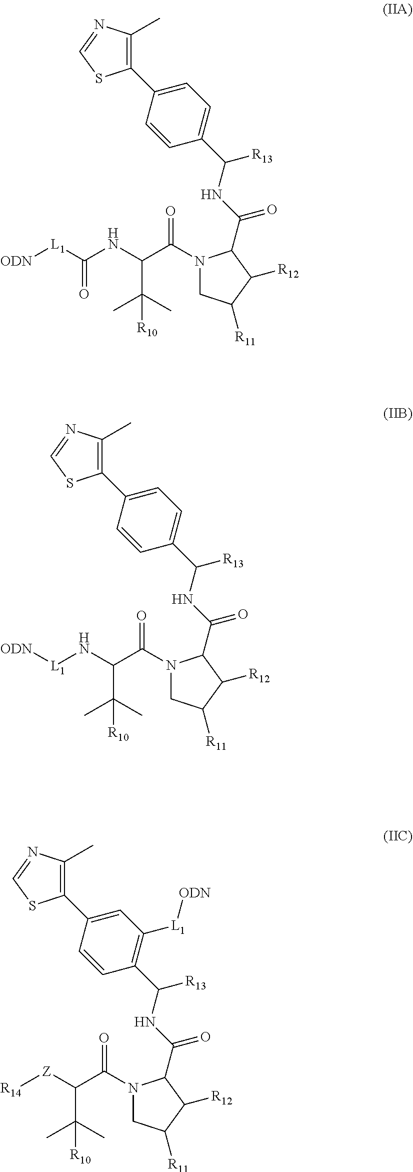

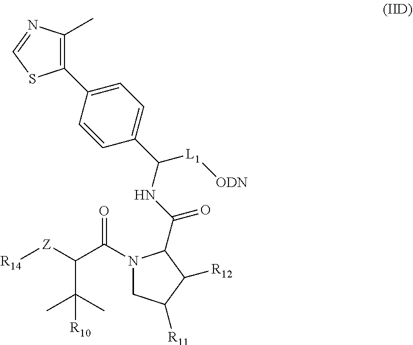

wherein Z is absent, —N(R^{a})—, —C(O)—, —C(O)N(R^{a})—, or —N(R^{a})C(O)—;
R₁₀ is hydrogen or alkyl (e.g., C₁-C₁₂ alkyl, C₁-C₈ alkyl, C₁-C₄ alkyl, methyl);
R₁₁ and R₁₂ are independently selected at each occurrence from hydrogen, alkyl (e.g., C₁-C₁₂ alkyl, C₁-C₈ alkyl, C₁-C₄ alkyl, methyl), halogen (e.g., F, Cl), and hydroxy;

R₁₃ is hydrogen or alkyl (e.g., C₁-C₁₂ alkyl, C₁-C₈ alkyl, C₁-C₄ alkyl, methyl);

R₁₄ is hydrogen, alkyl (e.g., C₁-C₁₂ alkyl, C₁-C₈ alkyl, C₁-C₄ alkyl, methyl), monocyclic or bicyclic C₃-C₁₂ cycloalkylene, monocyclic or bicyclic Cs-C₁₂ arylene, monocyclic or bicyclic 4 to 12 membered heteroalkylene, monocyclic or bicyclic 4 to 12 membered heteroarylene, each of which may be optionally substituted one or more times (e.g., with halogen, cyano, hydroxy, lower alkyl); and
R^{a} is independently at each occurrence hydrogen or lower alkyl (e.g., C₁-C₄ alkyl). In particular embodiments, R₁₄ is C₃-C₆ cycloalkyl optionally substituted with cyano or halogen (e.g., 1-cyanocyclopropyl), lower alkyl (e.g., C₁-C₄ alkyl, methyl), five or six membered aryl optionally substituted with, for example, alkyl, five or six membered heteroaryl (e.g., isoxazolyl) optionally substituted with, for example, alkyl (e.g., C₁-C₈ alkyl, C₁-C₄ alkyl, methyl), or a five or six membered heteroalkyl optionally substited with, for example, C═O fused to a fix or six membered aryl (e.g., oxoisoindolinyl such as 1-oxoisoindolin-2-yl) or a monocyclic or bicyclic heterocycle optionally unsaturated and optionally substituted with lower alkyl, halogen, C═O, cyano or hydroxy. In particular implementations, R₁₀ is hydrogen and/or Z is absent. In other embodiments, R₁₀ is lower alkyl (e.g., C₁-C₄ alkyl, methyl) and Z is —C(O)N(R^{a})—, or —N(R^{a})C(O)—. In various implementations the compound may have the structure of:

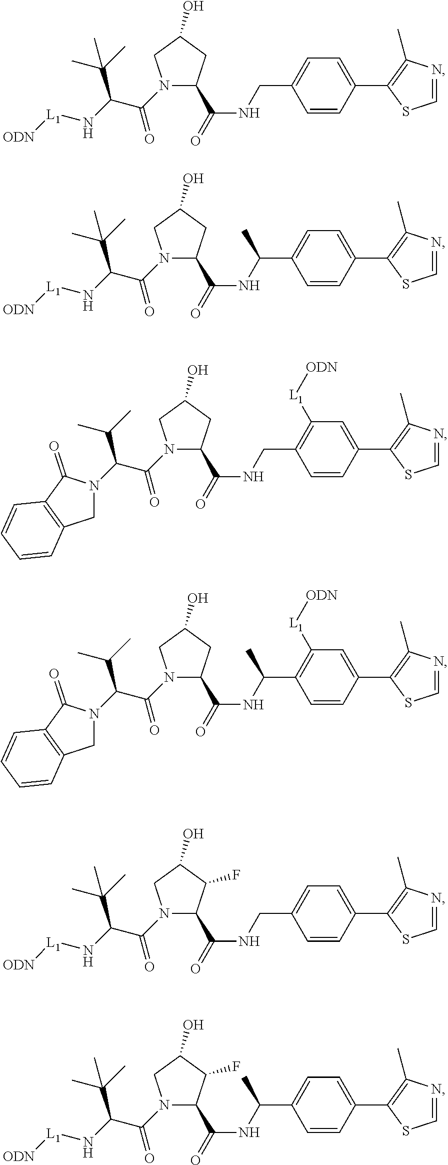

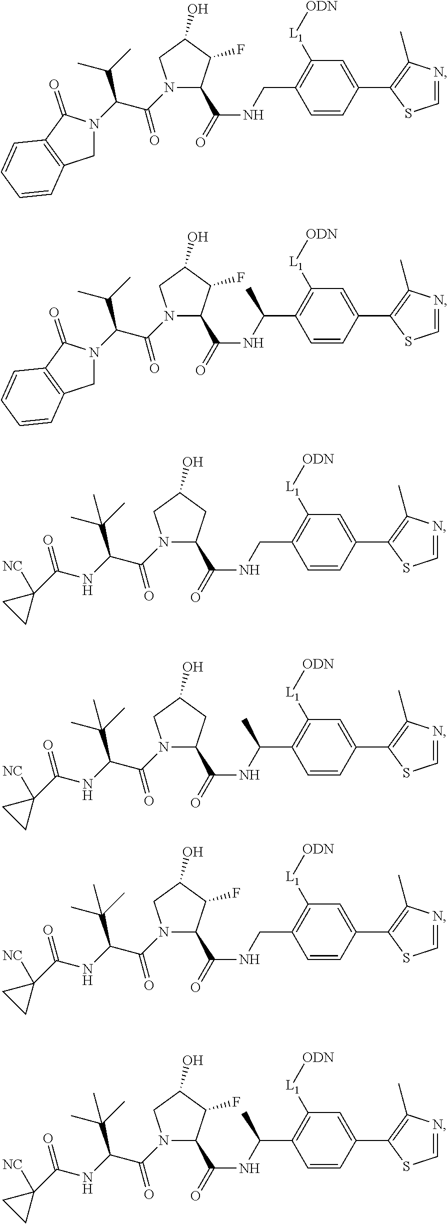

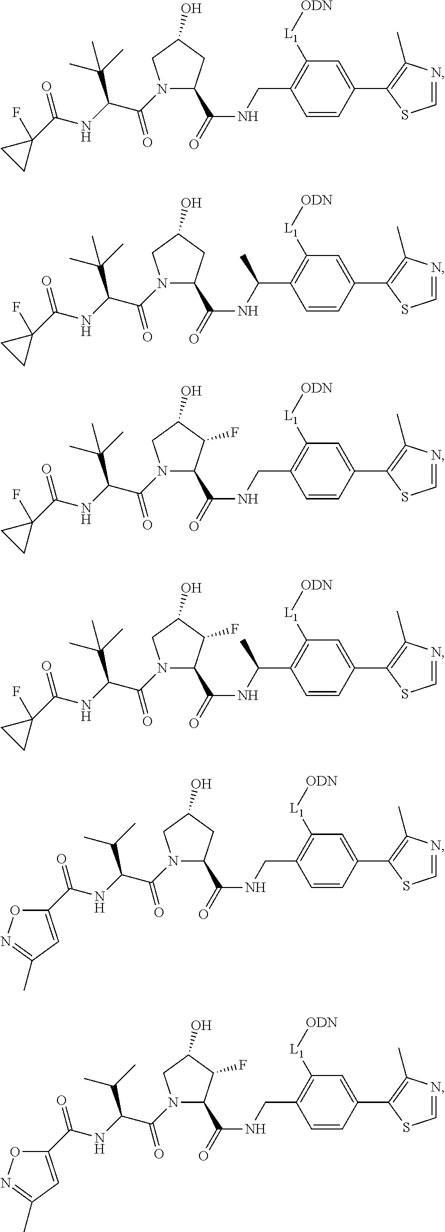

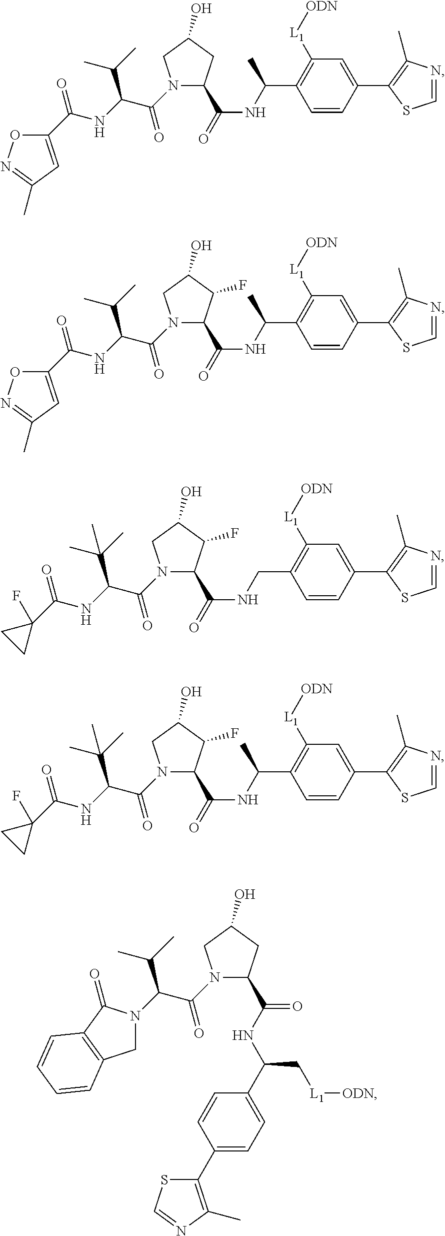

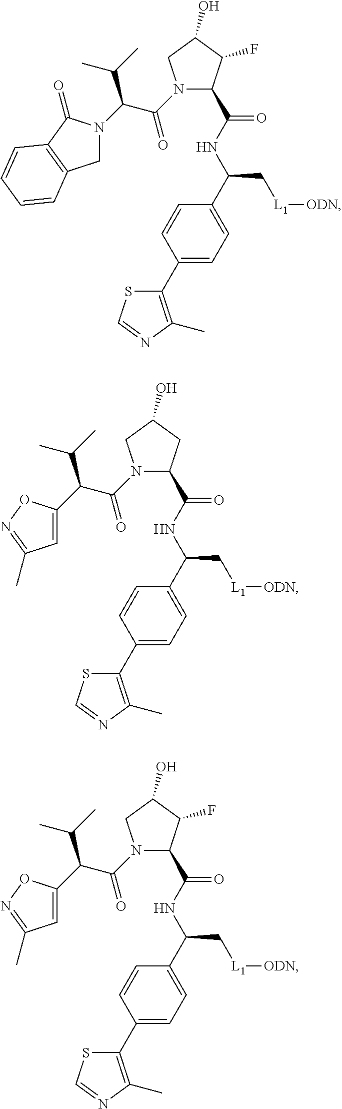

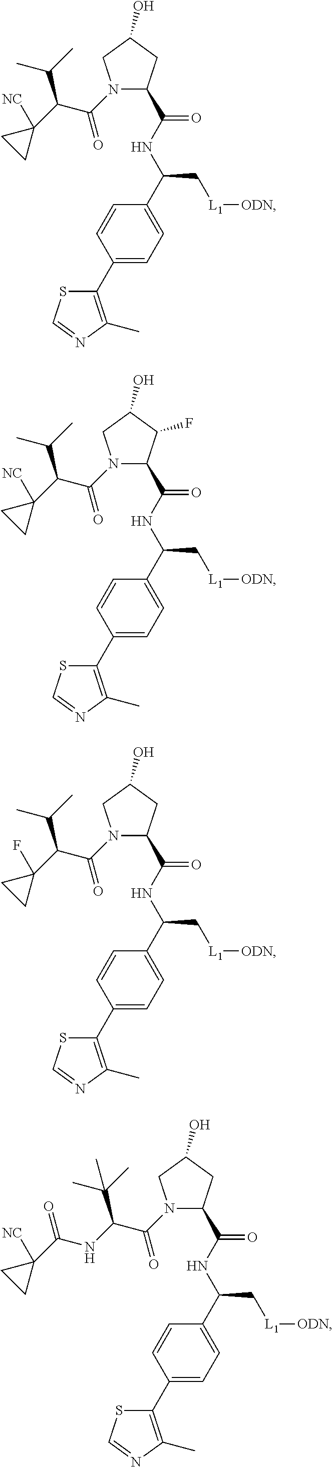

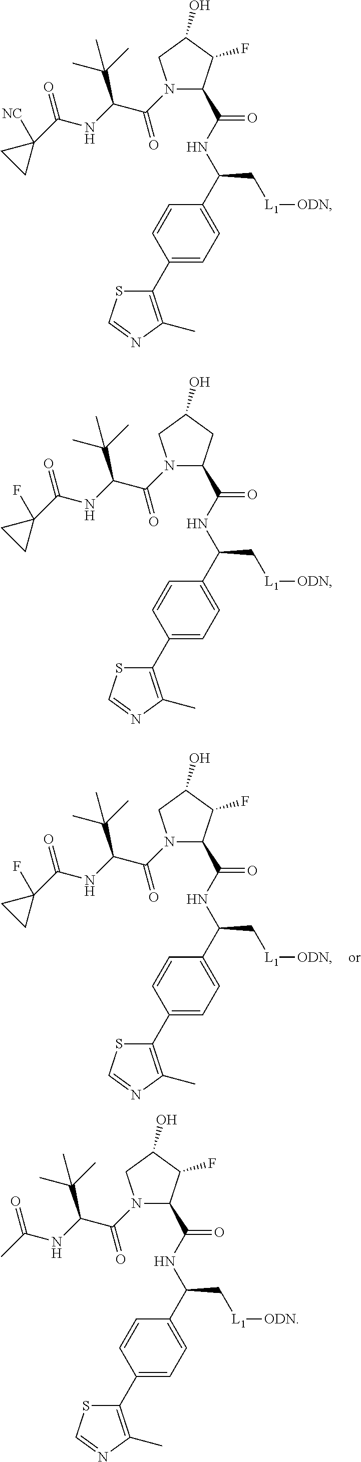

In some embodiments, the ULB has the structure of one of:

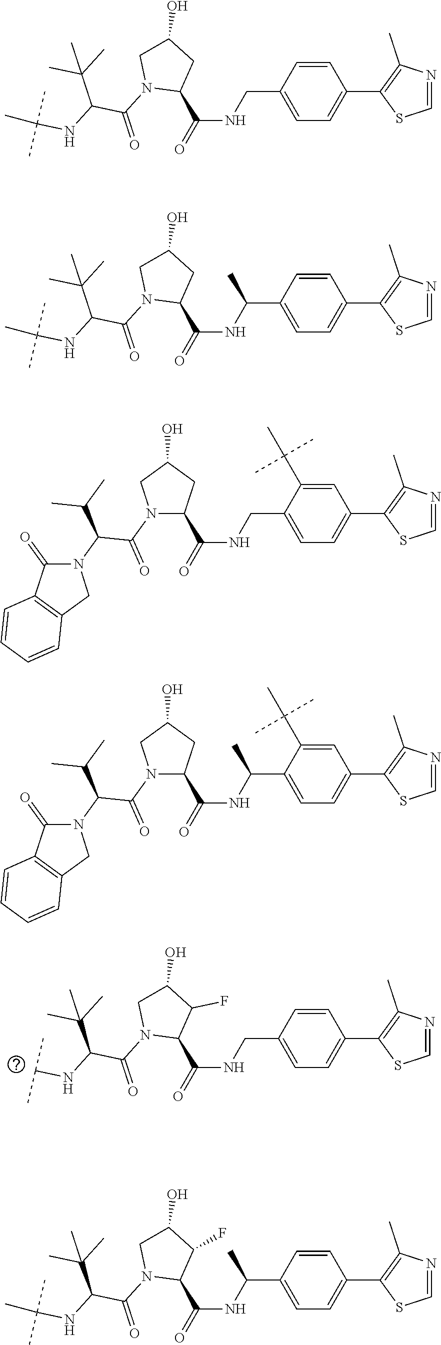

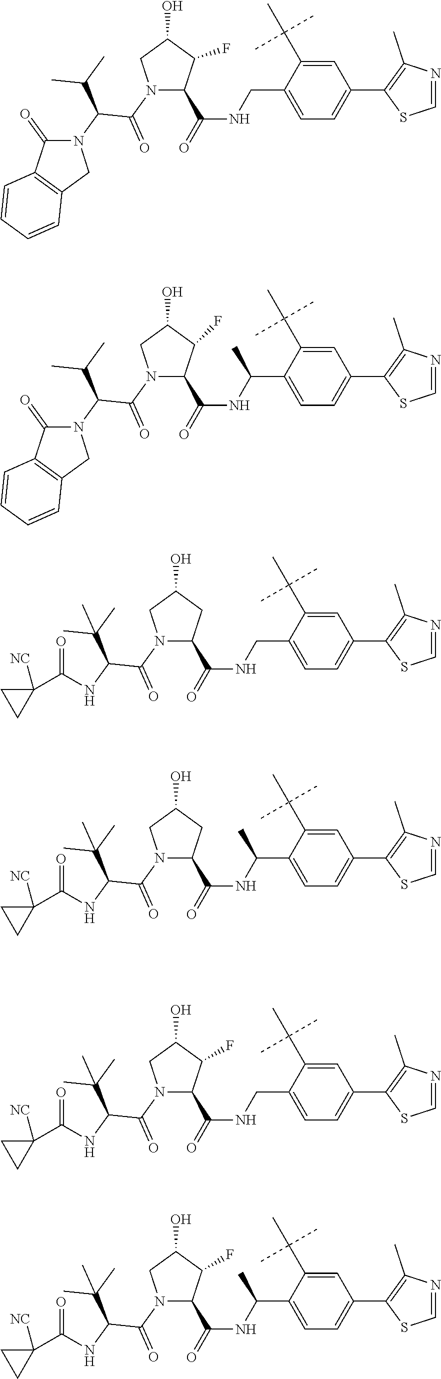

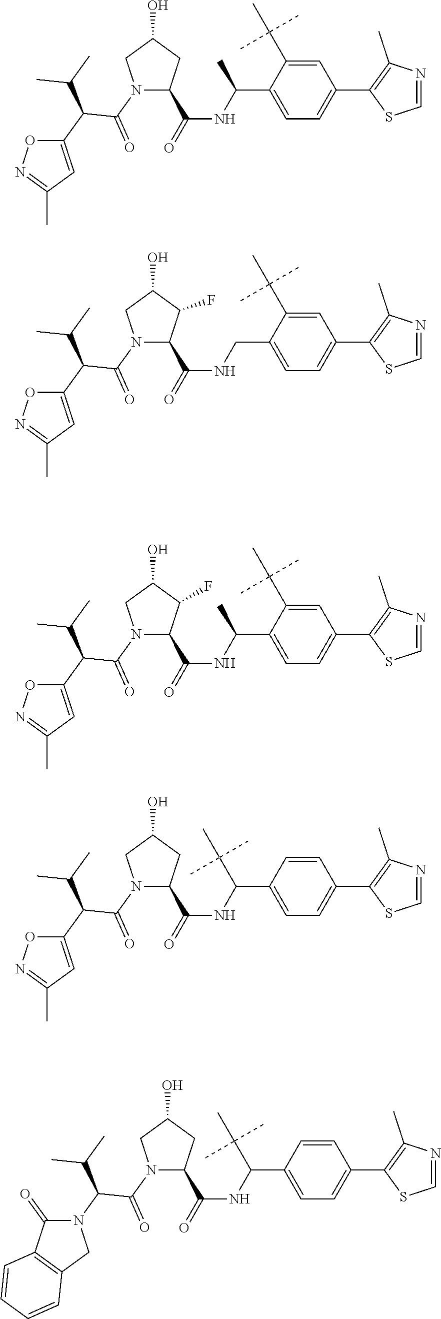

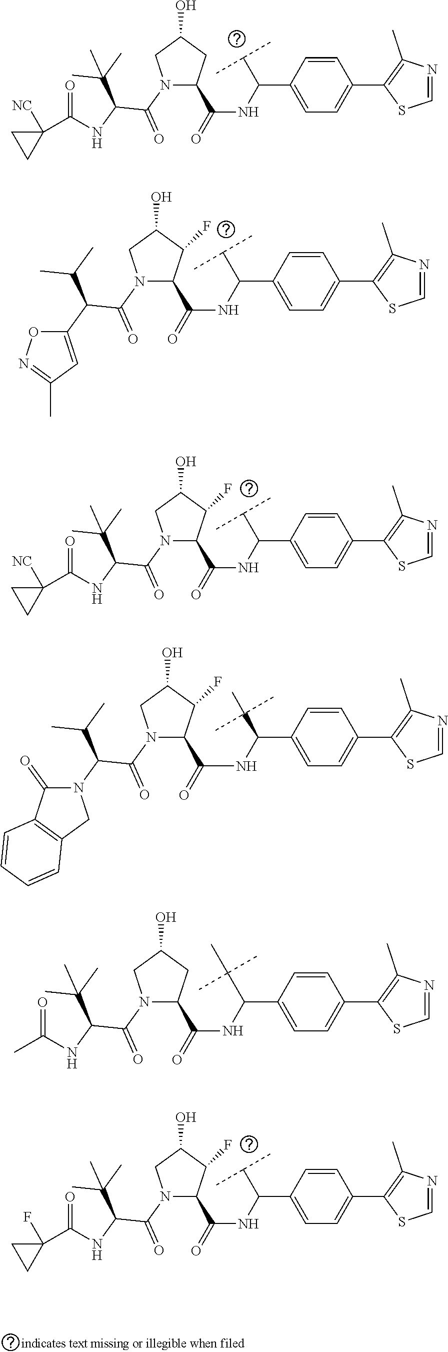

wherein

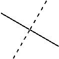

indicates the point of attachment to the L₁ group of formula (IV) or (I) (e.g., (formula (IVa), (IVb), (IVc), (IVd), (IVf), (IVg), (IVh), (IVi), (IVj), (IVk), (IVI), (IVm), (IVn), (V), (Va), (Vb), (Vc), (Vd), or (Ve)). In particular implementations, the compound has the structure of formula (II):

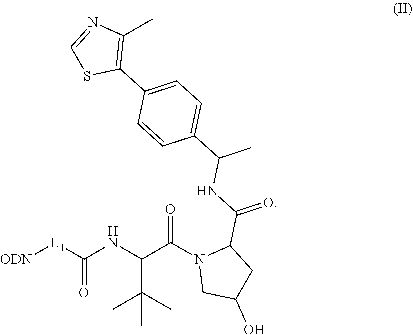

For example, the compound may have the structure of formula (IIa), (IIb), (IIc), (IId), or (IIe):

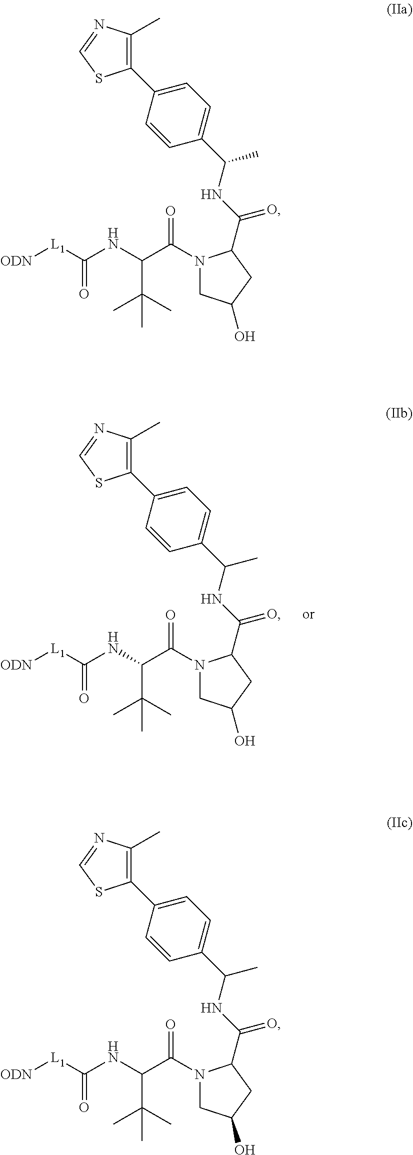

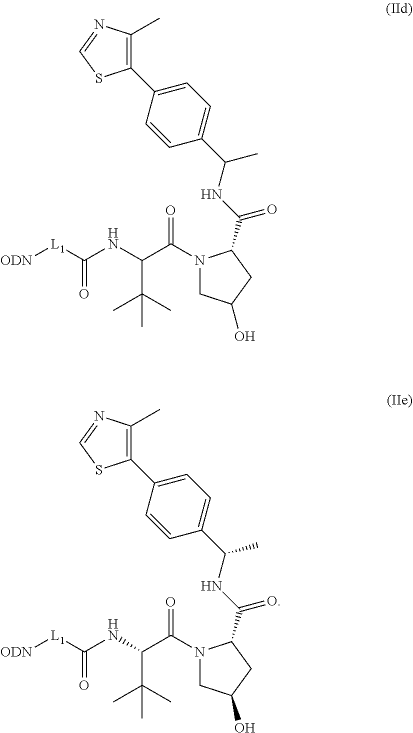

In some embodiments, the ULB is an immunomodulatory imide drug (IMiD) such as those and derivatives of those described in Ito, T. et al Science 327 (2010): 1345-1350, Kronke, J et al Science 343 (2014): 301-305, and Lu, G. et al Science 343 (2014): 305-309, Han, T. et al Science 356 (2017), and Uehara, T. et al Nat Chem Biol 13 (2017): 675-680, each of which are hereby incorporated by reference in their entirety and particularly in relation to sulfonamide based degraders. In some embodiments, the ULB is a cyclin K degrader such as those and derivatives of those described in Slabicki, M. et al Nature 585 (2020): 293-297, Mayor-Ruiz, C. et al. Nat Chem Biol 16 (2020): 1199-1207, and Lv, L. et al Elife 9 (2020): e59994, each of which are hereby incorporated by reference in their entirety and particularly in relation to cyclin K degraders. In some embodiments, ULB is lenalidomide derived (e.g., conjugated at two hydrogen positions of 3-(4-amino-1-oxoisoindolin-2-yl)piperidine-2,6-dione), pomalidomide derived (e.g., conjugated at two hydrogen positions of optionally substituted 4 -amino-2-(2,6-dioxopiperidin-3-yl)isoindoline-1,3-dione), or thalidomide derived (e.g., conjugated at two hydrogen positions of optionally substituted 2-(2,6-dioxopiperidin-3-yl)isoindoline-1,3-dione). In some embodiments, ULB comprises a piperidine dione.

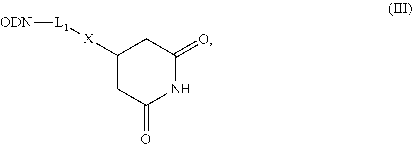

wherein X is absent (i.e, it is a bond) or may comprise the remaining portions of the ULB moiety including a five to ten membered mono or bicyclic optioanlly aromatic heterocycl optionally substituted with at least one or more (e.g., one, two) C═O groups (e.g., X may be optionally substituted isoindolin-1-one such as 4-aminoisoindolin-1-one, optionally substituted isoindolin, 1-3-dione such as 4-aminoisoindolin1,3,dione), wherein X is conjugated to the L₁ moiety (conjugation typically occurs through a hydrogen position of an unconjugated ULB). In some embodiments X is —R^{C}— or —R^{C}—R^{C}—, wherein R^{C} is independently selected at each occurrence from monocyclic or bicyclic C₃-C₁₂ cycloalkylene, monocyclic or bicyclic C₅-C₁₂ arylene, monocyclic or bicyclic 4 to 12 membered heteroalkylene, monocyclic or bicyclic 4 to 12 membered heteroarylene, each of which may be optionally substituted one or more times (e.g., with halogen, cyano, hydroxy, lower alkyl). In certain implementations, the compound has the structure of formula (IIIa):

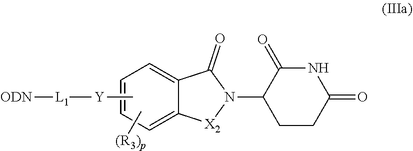

wherein p is 0, 1, 2, or, 3;
R₃ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.);
X₂ is C(O), CH₂, C(R^{a})₂, or NR^{a};
Y is absent (i.e, a bond), —C═C—, —O—, —C(O)—, —N(R^{a})—, —C(R^{a})₂—, OC(R^{a})₂—, —OC(O)—, —C(O)O—, —NR^{a}C(O)—, or —C(O)NR^{a}—;
R^{a} is independently selected at each occurrence from hydrogen, or alkyl (e.g., C₁-C₇ alkyl, C₁-C₄ alkyl, etc.). In some embodiments, Y is —N(R^{a})— and R^{a} is hydrogen or methyl. In various implementations, the ULB is lenalidomide derived, pomalidomide derived, or thalidomide derived. In various aspects, the ULB is lenalidomide derived and the compound has the structure of formula (IIIb) or (IIIc):

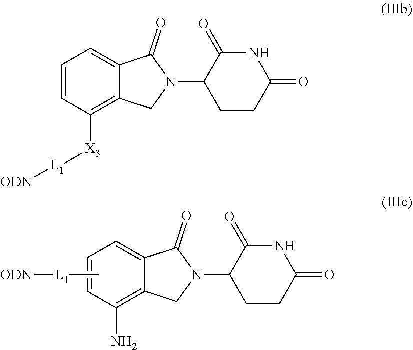

wherein X₃ is absent, —NH—, —N(R^{a})-(e.g., —N(CH₃)—), —C≡C—, or —O—.
In some embodiments, the ULB moiety is thalidomide derived and the compound has the structure of formula (IIId):

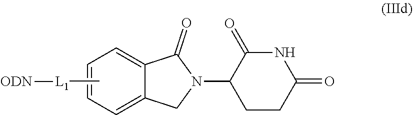

In specific embodiments, ULB moiety is pomalidomide derived and the compound has the structure of formula (IIIe) or (IIIf):

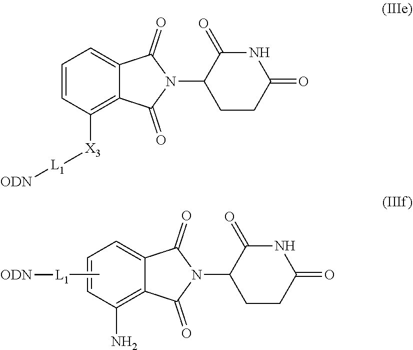

wherein X₃ is absent, —NH—, —N(R^{a})-(e.g., —N(CH₃)—), —C≡C—, or —O—. In some embodiments, the compound has the structure of formula (IIIg), (IIIh), (IIIi), or (IIj).

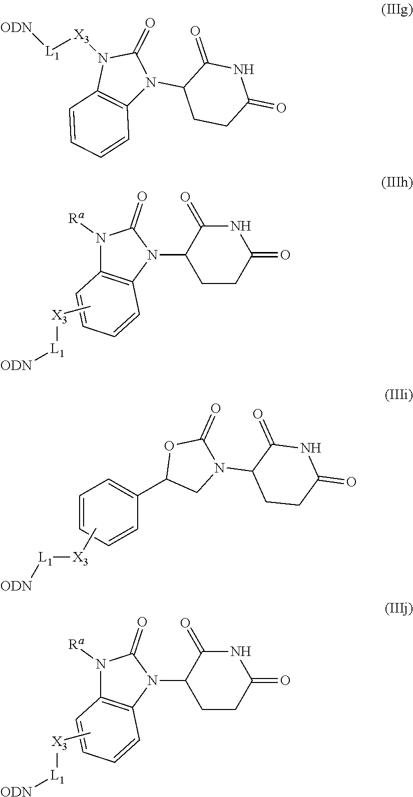

wherein X₃ is absent, —NH—, —N(R^{a})— (e.g., —N(CH₃)—), —C≡C—, or —O— and R^{a} is hydrogen or lower alkyl (e.g., C₁-C₄ alkyl).
In various implementations, the compound may have the structure of:

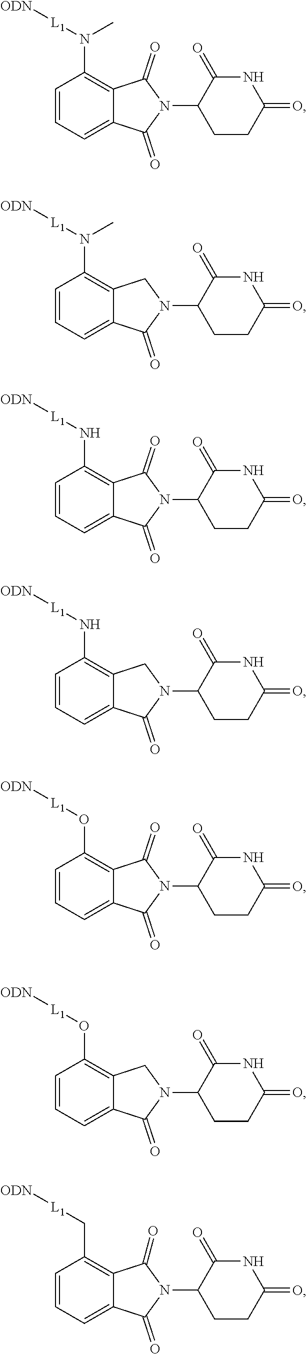

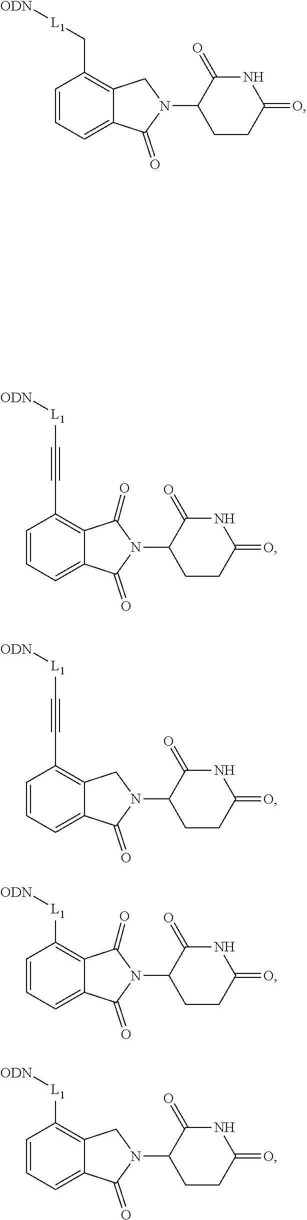

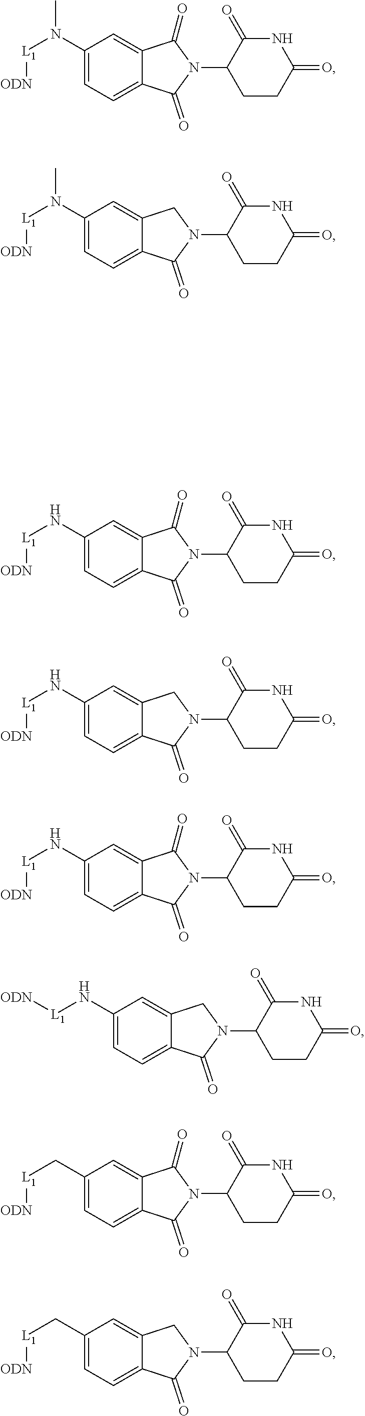

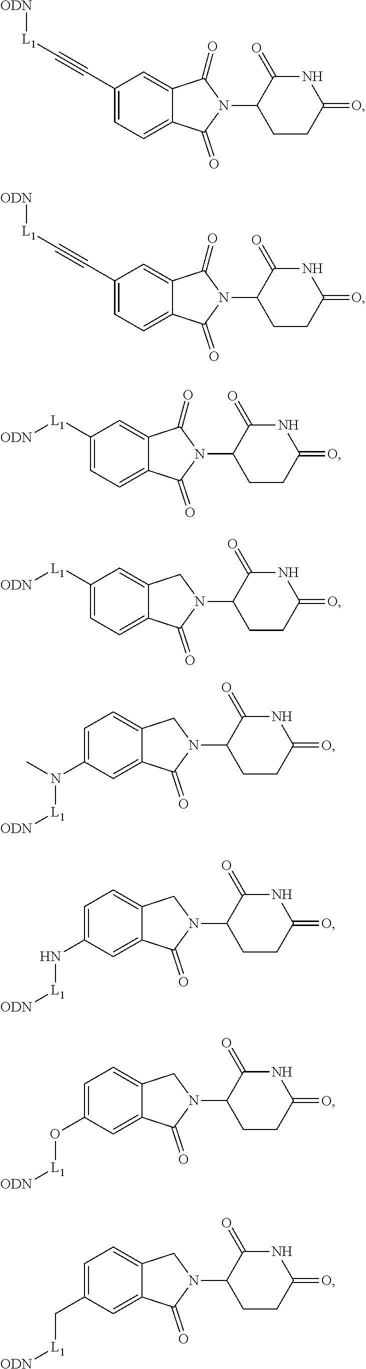

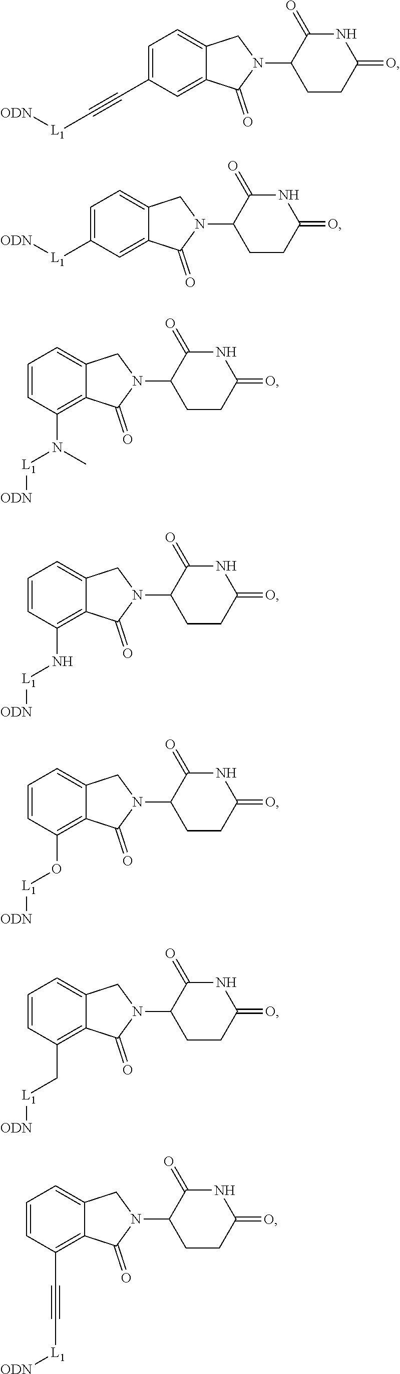

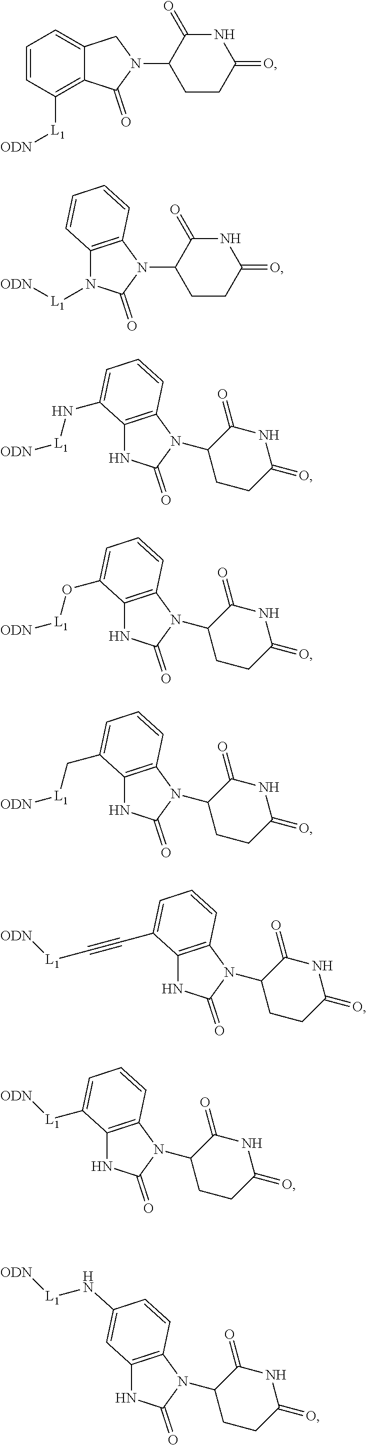

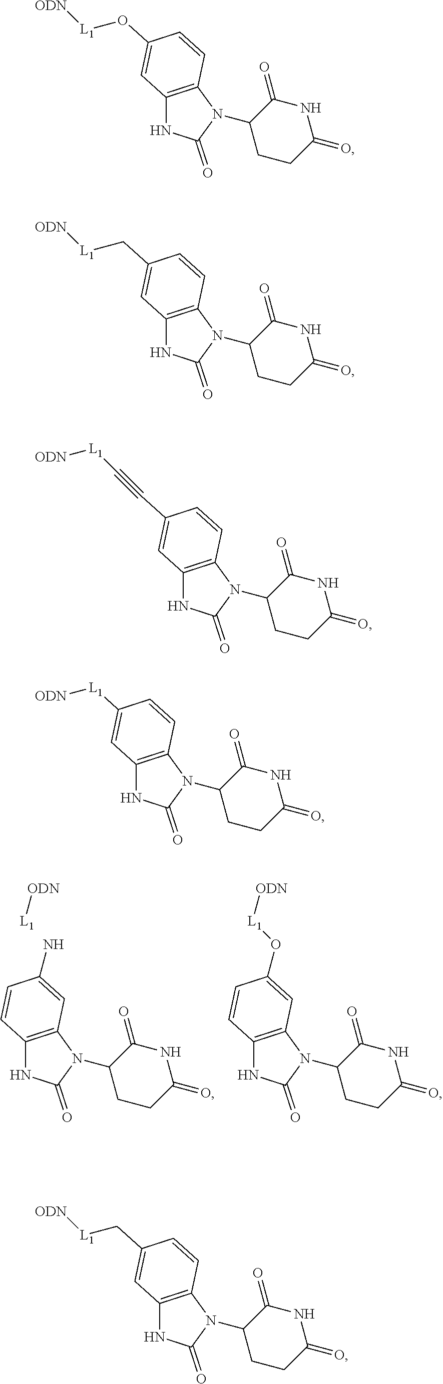

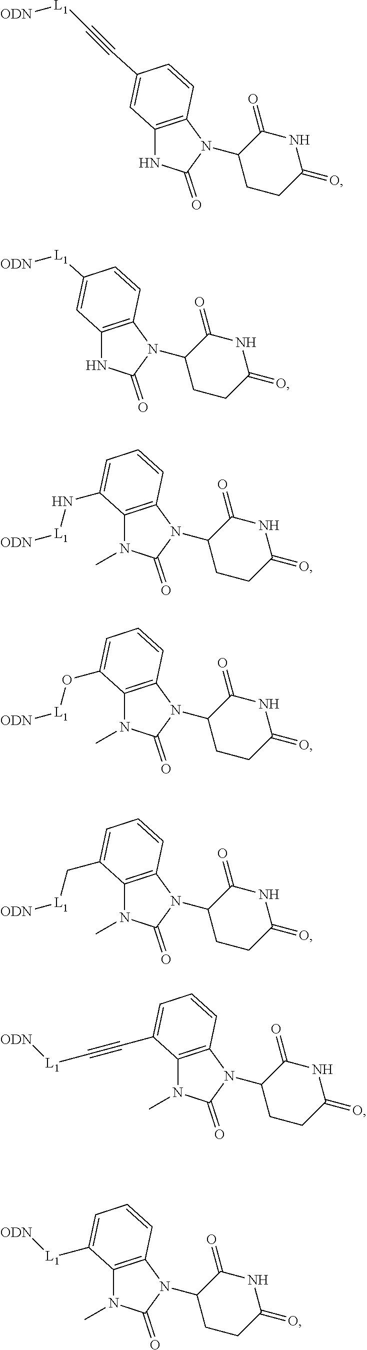

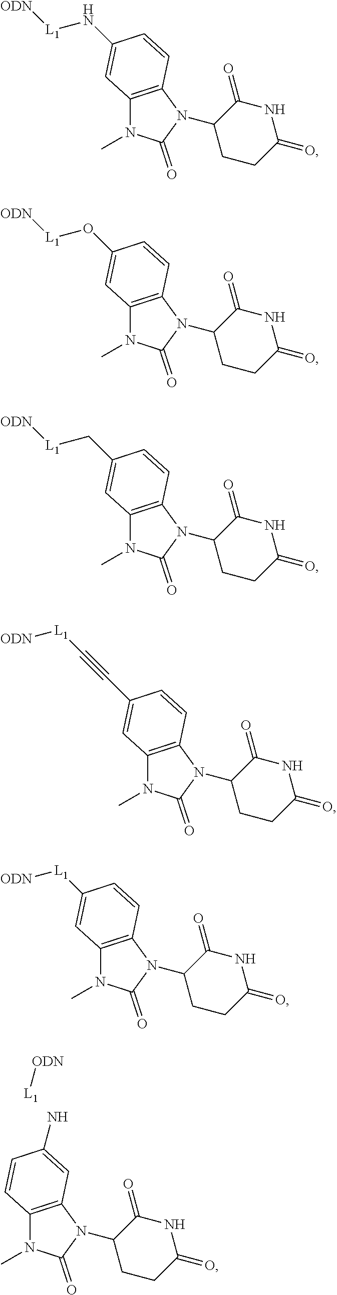

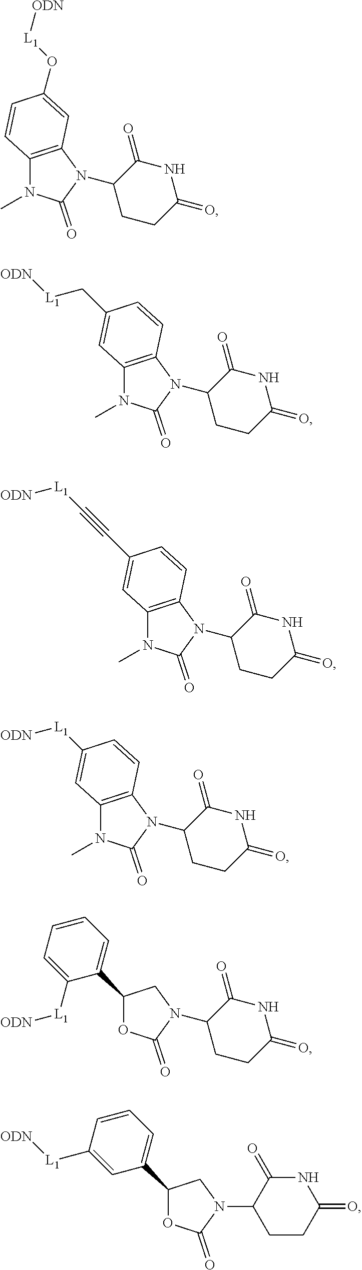

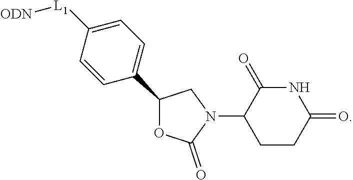

In various implementations, ULB has the structure of any one of:

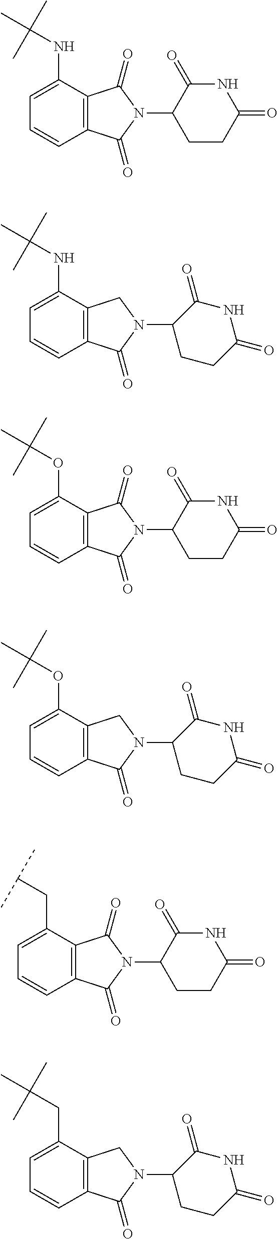

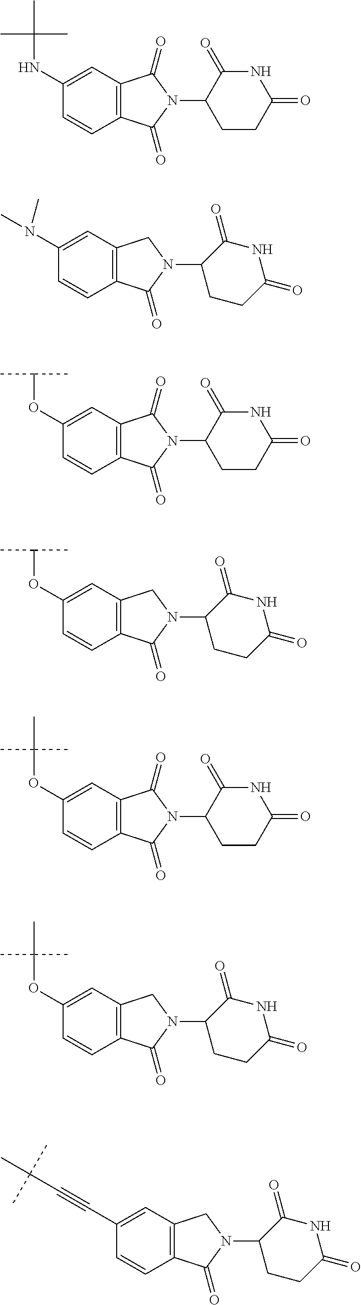

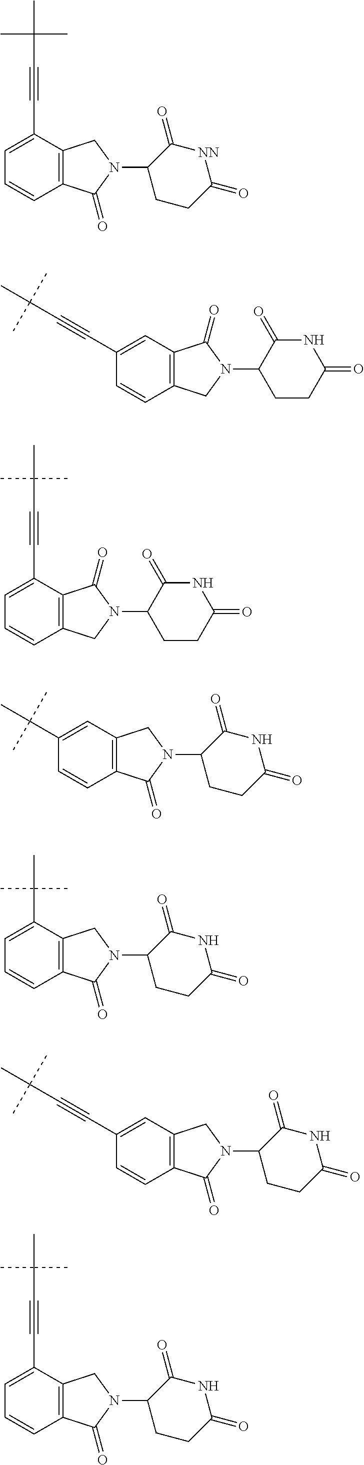

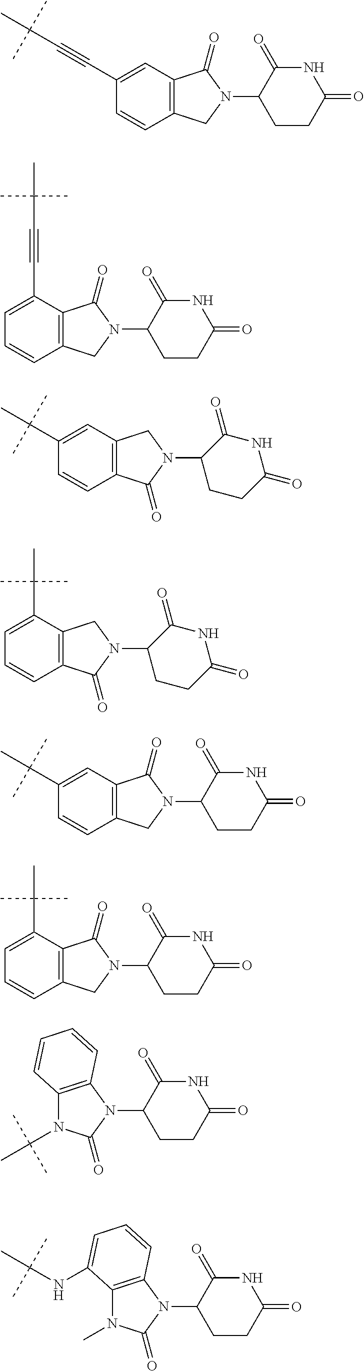

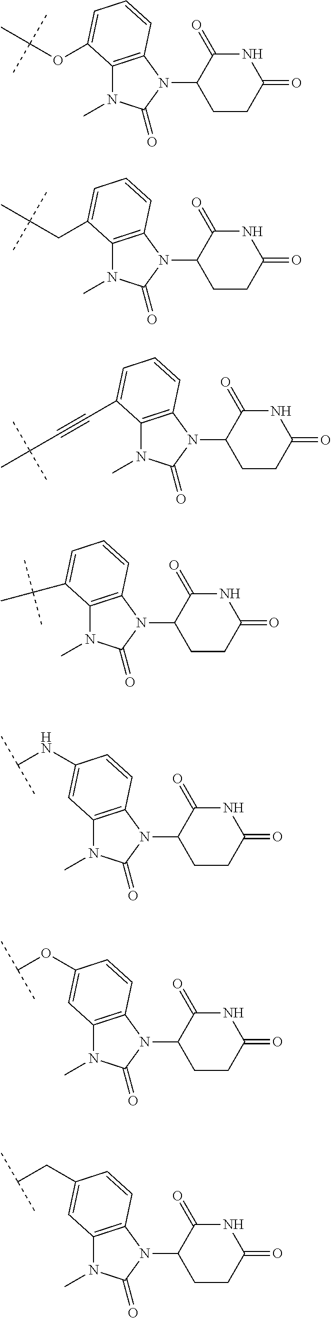

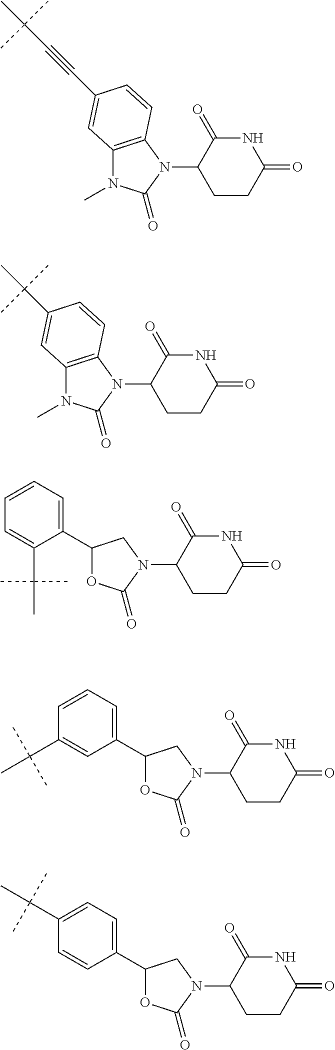

wherein

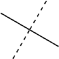

indicates the point of attachment to the L₁ group of formula (IV) or (I) (e.g., (formula (IVa), (IVb), (IVc), (IVd), (IVf), (IVg), (IVh), (IVi), (IVj), (IVk), (IVI), (IVm), (IVn), (V), (Va), (Vb), (Vc), (Vd), or (Ve)).

The ability to degrade the transcription factor may be strongly dependent on the linker between the chimera. In some embodiments, L₁ has the structure of formula (IV):

—Y₁—Y₂—Y₃—Y₄—Y₅—Y₆—Y₇—Y₈— (IV)

wherein Y₁-Y₈ are independently selected from absent, an optionally substituted heteroarylene group or optionally substituted heterocycloalkylene (e.g., a five membered heteroarylene, a six membered heteroarylene, a triazolylene, a imidazole, a divalent pyrrole) optionally fused to an mono or bicyclic cycloalkyl (e.g., C₃-C₁₀ monocyclic cycloalkyl, C₄-C₁₀ bicyclic cycloalkyl such as bicyclo[6.1.0] nonane) —C(O)—, —O—, —OC(O)—, —NR^{a}, —N(R^{a})C(O)—, —(C(R^{a})(R^{a}))₁₋₁₂, —(C(R^{a})(R^{a})C(R^{a})(R^{a})O)₁₋₁₂—, —S—S—; and
R^{a} is independently selected at each occurrence from hydrogen and optionally substitued alkyl (e.g., C₁-C₄ alkyl). In some embodiments, at least one of Y₁-Y₈ (e.g., Y_{2,} Y₃) comprises a linker moiety that is a click chemistry reaction product including click chemistry reaction products synthesized via another reaction mechanism. For example, at least one of Y₁-Y₈ may include a moiety that could be formed from Copper(I)-catalyzed azide-alkyne cycloaddition (CuAAC) reaction product (e.g., a divalent triazole), a strain-promoted azide-alkyne cycloaddition (SPAAC) product (e.g., a divalent fused triazole), a strain-promoted alkyne-nitrone cycloaddition (SPANC) product (e.g., a divalent fused dihydroisoxazole), or another product of strained alkene reactions such as alkene-azide cycloaddition. Click-chemistry compatible reactions (and products thereof for use as linker moieties in one of Y₁-Y₈) may also be considered to include alkene-tetrazine inverse-demand Diers-Alder reactions, alkene-tetrazole photoclick reactions, Michael additions of thiols, nucleophilic substitution of thiols with amines, and certain Diels-Alder reactions, such as those disclosed by Becer, et al. Angew. Chem. Int. Ed. 48 (2009): 4900-4908, which is hereby incorporated by reference in its entirety.

In some embodiments, one of Y₁-Y₈ includes a triazole product (e.g., similar to those produced from a Copper(I)-catalyzed azide-alkyne cycloaddition (CuAAC) reaction, or a Strain-promoted azide-alkyne cycloaddition (SPAAC)). In some embodiments, one of Y₁-Y₈ includes an isoxazole product such as fused dihydroisoxazole (e.g., similar to those produced from a Strain-promoted alkyne-nitrone cycloaddition (SPANC) reaction). In some embodiments, the compound has the structure of formula (IVa), (IVb), (IVc), (IVd), (IVf), (IVg), (IVh), (IVi), (IVj), (IVk), (IVI), (IVm), (IVn), (IVa1), (IVb1), (IVc1), (IVd1), (IVf1), (IVg1), (IVh1), (IVi1), (IVj1), (IVk1), (IVI1), (IVm1), (IVn1):

ODN—Y₁—Y₂—Y₃—NH—(CH₂)₁₋₁₂—NH—C(O)—ULB (IVa)

ODN—Y₁—Y₂—Y₃—(CH₂)₁₋₁₂—NH—C(O)—(CH₂)₁₋₁₂—ULB (IVb)

ODN—Y₁—Y₂—Y₃—(CH₂)₁₋₁₂—NH—(CH₂)₁₋₁₂—ULB (IVc)

ODN—Y₁—Y₂—Y₃—NH—(CH₂)₁₋₁₂—NH—C(O)—ULB (IVd)

ODN—Y₁—Y₂—Y₃—C(O)—(CH₂)₁₋₁₂—ULB (IVf)

ODN—Y₁—Y₂—Y₃—NH—(CH₂)₁₋₁₂—ULB (IVg)

ODN—Y₁—Y₂—Y₃—NH—(CH₂CH₂O)₁₋₁₂—NH—C(O)—ULB (IVh)

ODN—Y₁—Y₂—Y₃—(CH₂CH₂O)₁₋₁₂—NH—C(O)—(CH₂CH₂O)₁₋₁₂—ULB (IVi)

ODN—Y₁—Y₂—Y₃—(CH₂CH₂O)₁₋₁₂—NH—(CH₂CH₂O)₁₋₁₂—ULB (IVj)

ODN—Y₁—Y₂—Y₃—NH—(CH₂CH₂O)₁₋₁₂—NH—C(O)—ULB (IVk)

ODN—Y₁—Y₂—Y₃—C(O)—(CH₂CH₂O)₁₋₁₂—ULB (IVl)

ODN—Y₁—Y₂—Y₃—NH—(CH₂CH₂O)₁₋₁₂—ULB (IVm)

ODN—Y₁—Y₂—Y₃—(CH₂)₁₋₁₂—C(O)—NH—(CH₂)₁₋₁₂—ULB (IVn)

ODN—Y₁—Y₂—Y₃—(CH₂)₁₋₁₂—OC(O)—NH—(CH₂)₁₋₁₂—ULB (IVo)

ODN—Y₁—Y₂—Y₃—(CH₂)₁₋₁₂—OC(O)—NH—(CH₂CH₂O)₁₋₁₂—ULB (IVp)

ODN—Y₁—Y₂—Y₃—(CH₂)₁₋₁₂—OC(O)—NH—(CH₂)₁₋₁₂—(CH₂CH₂O)₁₋₁₂—ULB (IVq)

ODN—Y₁—Y₂—Y₃—(CH₂)₁₋₁₂—OC(O)—NH—(CH₂CH₂O)₁₋₁₂—(CH₂)₁₋₁₂—ULB (IVr)

wherein Y₁-Y₈ are independently selected from absent, an optionally substituted heterocycloalkylene or heteroarylene group (e.g., a five membered heteroarylene, a six membered heteroarylene, a divalent triazole, a divalent imidazole, a divalent pyrrole) optionally fused to an mono or bicyclic cycloalkyl (e.g., C₃-C₁₀ monocyclic cycloalkyl, C₄-C₁₀ bicyclic cycloalkyl such as bicyclo[6.1.0] nonane), —C(O)—, —O—, —OC(O)—, —NR^{a}—, —N(R^{a})C(O)—, —(C(R^{a})(R^{a}))1-12, -(C(R^{a})(R^{a})C(R^{a})(R^{a})O)₁₋₁₂—, and —S—S—; and
R^{a} is independently selected at each occurrence from hydrogen and optionally substitued alkyl (e.g., C₁-C₄ alkyl). In various implementations, the compound has the structure of formula (IVa1), (IVb1), (IVc1), (IVd1), (IVf1), (IVg1), (IVh1), (IVil), (IVj1), (IVk1), (IVl1), (IVm1), or (IVn1):

ODN—NHCO—(CH₂)₁₋₁₂—Y₃—NH—(CH₂)₁₋₁₂—NH—C(O)—ULB (IVa1)

ODN—NHCO—(CH₂)₁₋₁₂—Y₃—(CH₂)₁₋₁₂—NH—C(O)—(CH₂)₁₋₁₂—ULB (IVb1)

ODN—NHCO—(CH₂)₁₋₁₂—Y₃—(CH₂)₁₋₁₂—NH—(CH₂)₁₋₁₂—ULB (IVc1)

ODN—NH—(CH₂)₁₋₁₂—Y₃—NH—(CH₂)₁₋₁₂—NH—C(O)—ULB (IVd1)

ODN—NH—(CH₂)₁₋₁₂—Y₃—C(O)—(CH₂)₁₋₁₂—ULB (IVf1)

ODN—NH—(CH₂)₁₋₁₂—Y₃—NH—(CH₂)₁₋₁₂—ULB (IVg1)

ODN—NHCO—(CH₂)₁₋₁₂—Y₃—NH—(CH₂CH₂₀)₁₋₁₂—NH—C(O)—ULB (IVh1)

ODN—NHCO—(CH₂)₁₋₁₂—Y₃—(CH₂CH₂₀)₁₋₁₂—NH—C(O)—(CH₂CH₂₀)₁₋₁₂—ULB (IVi1)

ODN—NHCO—(CH₂)₁₋₁₂—Y₃-(CH₂CH20)₁₋₁₂—NH—(CH₂CH20)1-12-ULB (IVj1)

ODN—NHCO—(CH₂)₁₋₁₂—Y₃—NH—(CH₂CH₂₀)₁₋₁₂—NH—C(O)—ULB (IVk1)

ODN—NHCO—(CH₂)₁₋₁₂—Y₃—C(O)—(CH₂CH₂O)₁₋₁₂—ULB (IVl1)

ODN—NHCO—(CH₂)₁₋₁₂—Y₃—NH—(CH₂CH₂₀)₁₋₁₂—ULB (IVm1)

ODN—NHCO—(CH₂)₁₋₁₂—Y₃—(CH₂)₁₋₁₂—C(O)—NH—(CH₂)₁₋₁₂—ULB (IVn1)

ODN—NHCO—(CH₂)₁₋₁₂—Y₃—(CH₂)₁₋₁₂—OC(O)—NH—(CH₂)₁₋₁₂—ULB (IVo1)

ODN—NHCO—(CH₂)₁₋₁₂—Y₃—(CH₂)₁₋₁₂—OC(O)—NH—(CH₂CH20)₁₋₁₂—ULB (IVp1)

ODN—NHCO—(CH₂)₁₋₁₂—Y₃—(CH₂)₁₋₁₂—OC(O)—NH—(CH₂)₁₋₁₂-(CH₂CH₂₀)₁₋₁₂—ULB (IVq1)

ODN—NHCO—(CH₂)₁₋₁₂—Y₃—(CH₂)₁₋₁₂—OC(O)—NH—(CH₂CH20)₁₋₁₂—(CH₂)₁₋₁₂—ULB (IVr1)

Particularly due to synthetic routes involving azide/alkynyl cycloaddition, in certain embodiments, Y₂ or Y₃ may be optionally substituted heterocycloalkylene or heteroarylene group (e.g., a five membered heteroarylene, a six membered heteroarylene, a divalent triazole, a divalent imidazole, a divalent pyrrole) optionally fused to an mono or multicyclic cycloalkyl (e.g., C₃-C₁₀ monocyclic cycloalkyl, C₄-C₁₀ bicyclic cycloalkyl such as bicyclo[6.1.0] nonane).

For example, at least one of Y₁—Y₇ (e.g., Y₂, Y₃) may be a linker thaty may be formed from a strained click chemistry reaction (also referred to herein as —L_{SCC}—) such as those having the structure:

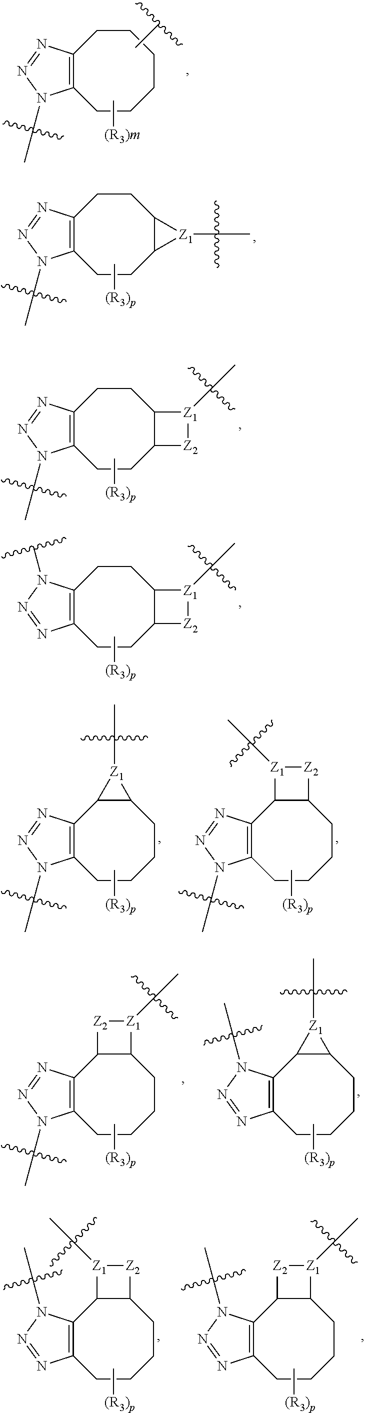

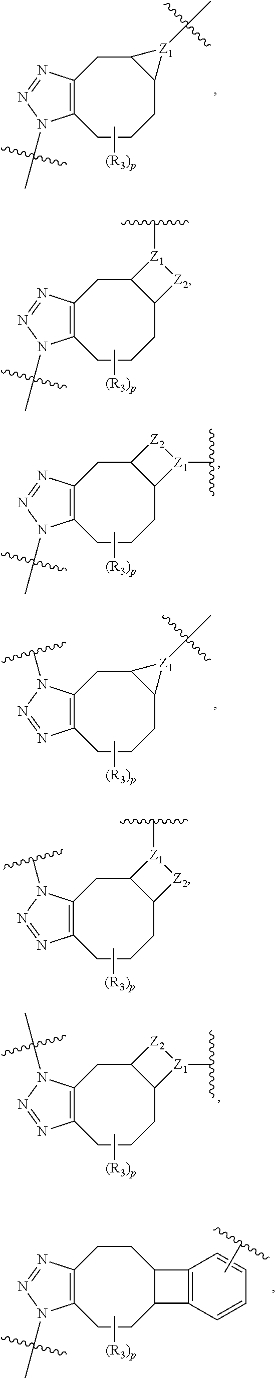

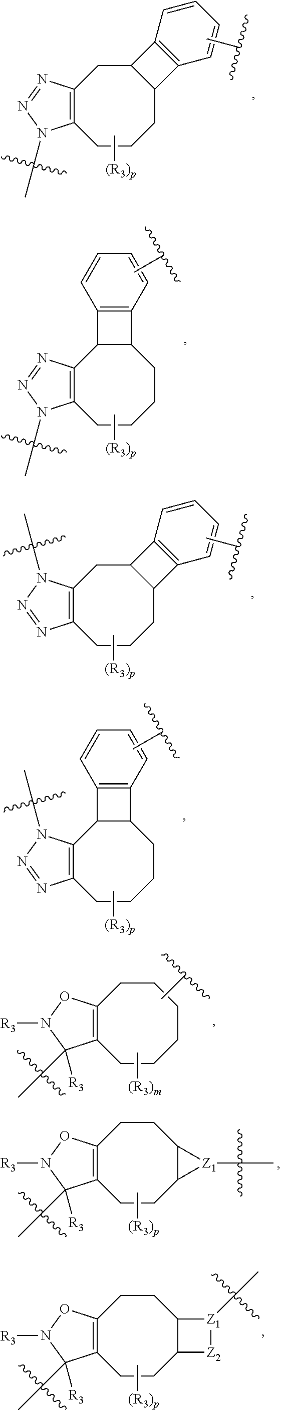

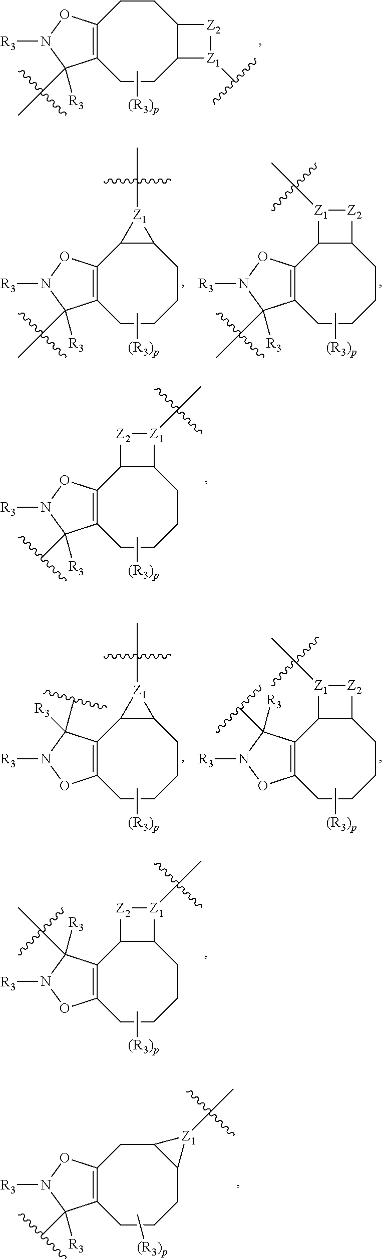

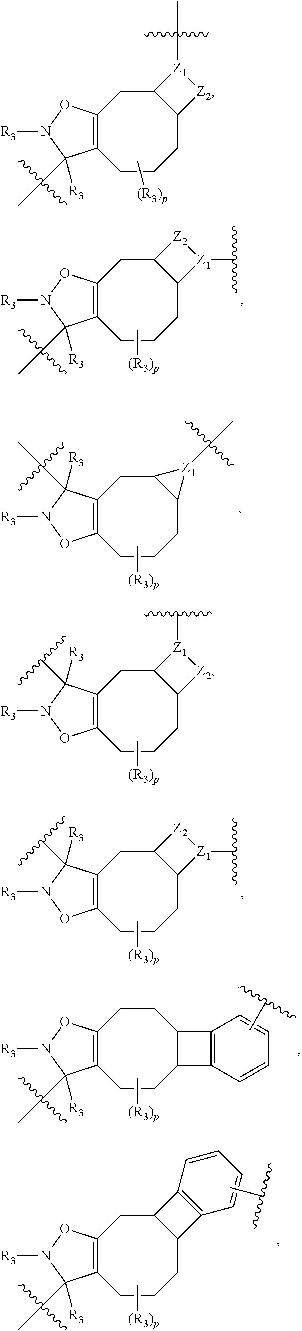

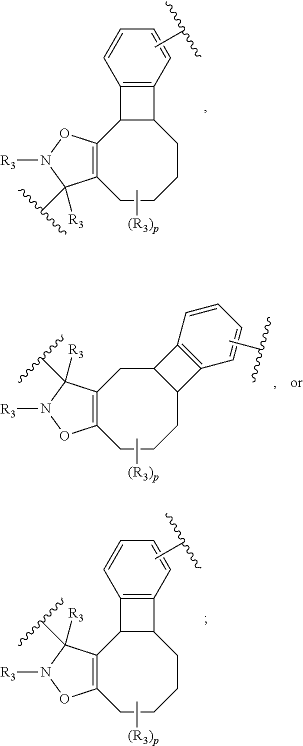

wherein each

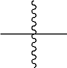

indicates the point of attachment to the neighboring linker group (e.g., Y₁, Y₂, Y₃, Y₄);
m is an integer from 0-12 (0, 1, 2, 3, 4, 5, 6, 7, 8, 9, 10, 11, or 12);
p is an integer from 0-10 (0, 1, 2, 3, 4, 5, 6, 7, 8, 9, or 10);
Z₁ is —N— or —CR₃—;
Z₂ is O, C(O), or C(R₃)₂; and

R₃ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.) and wherein any two vicinal R₃ groups of the Cs ring may together form a five or six membered optionally substituted optionally aromatic ring (e.g., aryl including phenyl) fused to the Cs ring. In some embodiments, at least one of Y₁—Y₇ (e.g., Y₂, Y₃) comprises the structure:

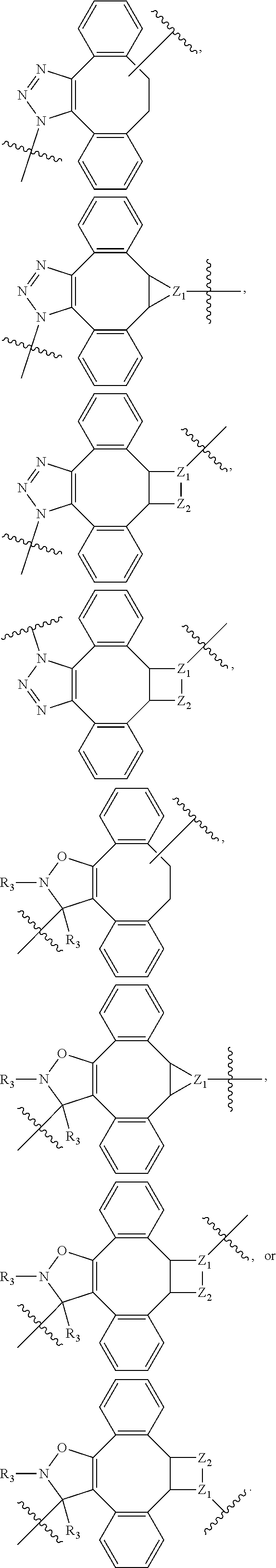

In particular embodiments, the compound has the structure of formula (V):

ODN—Y₁—Y₂—L_{SCC}—Y₄—Y₅—Y₆—Y₇—Y₈—ULB. (V)

For example, the compound of the present disclosure may have the structure:

ODN—NHCO—(CH₂)₁₋₁₂—L_{SCC}—NH—(CH₂)₁₋₁₂—NH—C(O)—ULB

ODN—NHCO—(CH₂)₁₋₁₂—L_{SCC}—(CH₂)₁₋₁₂—NH—C(O)—(CH₂)₁₋₁₂—ULB

ODN—NHCO—(CH₂)₁₋₁₂—L_{SCC}—(CH₂)₁₋₁₂—NH—(CH₂)₁₋₁₂—ULB

ODN—NH—(CH₂)₁₋₁₂—L_{SCC}—NH—(CH₂)₁₋₁₂—NH—C(O)—ULB

ODN—NH—(CH₂)₁₋₁₂—L_{SCC}—C(O)—(CH₂)₁₋₁₂—ULB

ODN—NH—(CH₂)₁₋₁₂—L_{SCC}—NH—(CH₂)₁₋₁₂—ULB

ODN—NHCO—(CH₂)₁₋₁₂—L_{SCC}—NH—(CH₂CH₂₀)₁₋₁₂—NH—C(O)—ULB

ODN—NHCO—(CH₂)₁₋₁₂—L_{SCC}—(CH₂CH₂₀)₁₋₁₂—NH—C(O)—(CH₂CH₂₀)₁₋₁₂—ULB

ODN—NHCO—(CH₂)₁₋₁₂—L_{SCC}—(CH₂CH₂₀)₁₋₁₂—NH—(CH₂CH₂₀)₁₋₁₂—ULB

ODN—NHCO—(CH₂)₁₋₁₂—L_{SCC}—NH—(CH₂CH₂₀)₁₋₁₂—NH—C(O)—ULB

ODN—NHCO—(CH₂)₁₋₁₂—L_{SCC}—C(O)—(CH₂CH₂O)₁₋₁₂—ULB

ODN—NHCO—(CH₂)₁₋₁₂—L_{SCC}—NH—(CH₂CH₂₀)₁₋₁₂—ULB

ODN—NHCO—(CH₂)₁₋₁₂—L_{SCC}—(CH₂)₁₋₁₂—C(O)—NH—(CH₂)₁₋₁₂—ULB

ODN—NHCO—(CH₂)₁₋₁₂—L_{SCC}—(CH₂)₁₋₁₂—OC(O)—NH—(CH₂)₁₋₁₂—ULB

ODN—NHCO—(CH₂)₁₋₁₂—L_{SCC}—(CH₂)₁₋₁₂—OC(O)—NH—(CH₂CH₂₀)₁₋₁₂—ULB

ODN—NHCO—(CH₂)₁₋₁₂—L_{SCC}—(CH₂)₁₋₁₂—OC(O)—NH—(CH₂)₁₋₁₂—(CH₂CH₂O)₁₋₁₂—ULB

ODN—NHCO—(CH₂)₁₋₁₂—L_{SCC}—(CH₂)₁₋₁₂—OC(O)—NH—(CH₂CH₂₀)₁₋₁₂—(CH₂)₁₋₁₂—ULB

The compounds of the present disclosure may have a linker length and be characterized with a click efficiency (e.g., as measured by reaction between a synthetic intermediate of the present disclosure with 10 fold excess of an azide linked ODN group) may be above 10%, above 20% or above 30% or above 40% or above 60% or above 70% or above 80% or above 90% (e.g., from 10%-99%, from 20%-99%, from 30%-99%, 40%-99%, 50%-99%, 60%-99%, 70%-99%, from 10%-99%, from 20%-99%, from 30%-99%, 40%-99%, 50%-99%, 60%-99%, 70%-99%, from 20%-90%, from 30%-90%, 40%-90%, 50%-90%, 60%-90%, 70%-90%, from 20%-90%, from 30%-90%, 40%-90%, 50%-90%, 60%-90%, 70%-90%) efficient.

In some embodiments, the compound may have the structure of formula (Va):

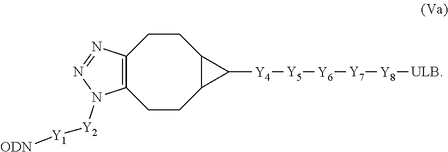

The Y₄-Y₈ may chosen to alter the activity of the PROTAC. For example, in some embodiments, the compound has the structure of formula (Vb), (Vc), (Vd), or (Ve):

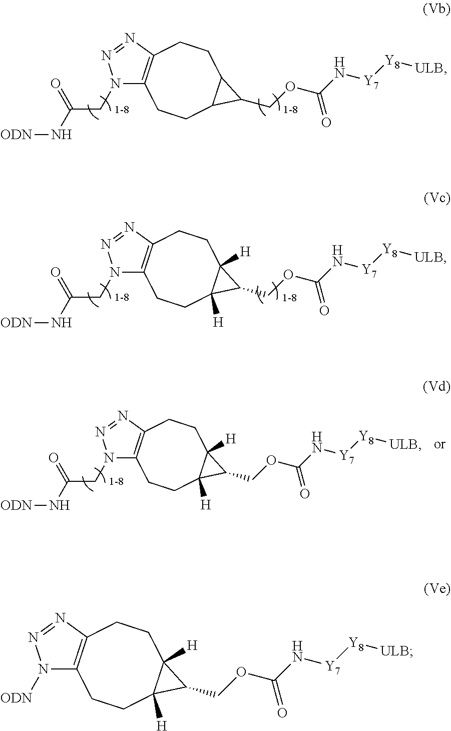

wherein Y₇ and Ys are independently selected from absent (i.e., it is a bond), —(C(R^{a})(R^{a}))₁₋₁₂, and —(C(R^{a})(R^{a})C(R^{a})(R^{a})O)₁₋₁₂—; and
R^{a} is independently selected at each occurrence from hydrogen and optionally substitued alkyl (e.g., C₁-C₄ alkyl). In some embodiments, Y₈ is absent and Y₇ is —(CH₂)₁₋₁₂. In various implementations, Y₇is —(CH₂CH₂₀)₁₋₁₂— and Y₈ is —(CH₂)₁₋₁₂. In certain embodiments, —Y₇—Y₈— (particulalry in compounds having the structure of formula (Va), (Vb), (Vc), (Vd), or (Ve)) may be part of a linker as shown in Table 1.

TABLE 1

| Linker Number | —Y₇—Y₈— |
|---|---|
| #1 | —CH₂— |
| #2 | —(CH₂)₂— |
| #3 | —(CH₂)₃— |
| #4 | —(CH₂)₄— |
| #5 | —(CH₂)₅— |
| #6 | —(CH₂)₆— |
| #7 | —(CH₂)₇— |
| #8 | —(CH₂)₈— |
| #9 | —(CH₂)₉— |
| #10 | —(CH₂)₁₀— |
| #11 | —CH₂CH₂OCH₂— |
| #12 | —(CH₂CH₂O)₂CH₂— |
| #13 | —(CH₂CH₂O)₃CH₂— |
| #14 | —CH₂CH₂O(CH₂)₂— |
| #15 | —(CH₂CH₂O)₂(CH₂)₂— |
| #16 | —(CH₂CH₂O)₃(CH₂)₂— |
| #17 | —(CH₂CH₂O)₄(CH₂)₂— |
| #18 | —(CH₂CH₂O)₅(CH₂)₂— |

In particular embodiments, the compound comprises linkers 4, 7, 15, 16, or 18 from Table 1 or FIG. 7A. References to dNF-κB and dE2F by linker number may refer to linkers as identified in Table 1 or FIG. 7A.

The selection of the ODN targetting factor is typically guided by those DNA oligomer protein sequences (e.g., double stranded DNA oligomers) which bind to a transcription factor binding motif. For example, the ODN may bind to NF-κB or E2F. In some embodiments, ODN binds to the p65 subunit protein. In certain implementations, ODN binds to the E2F1 subunit protein. In various aspects, ODN has a double-band hairpin structure optionally formed via intra-dimerization. In some embodiments, ODN is a double-stranded DNA comprising a sense chain and an anti-sense chain.

The DNA oligomer protein of ODN may have from 1-30 bases (e.g., 10-20 bases, 1-10 bases, 20-30 bases) and comprises transcription factor binding motif. In some embodiments, the transcription factor binding motif is GGGRNNYYCC (SEQ ID NO: 1) or TTTG/CG/CCGC; wherein R is purine, Y is pyrimidine, and N is any base. ODN may comprise (or is) the sequence

(SEQ ID NO: 2)
ACGGACCGGAAATCCGGTT,
(SEQ ID NO: 3)
TACAAAGATCAAAGGGTT,
ATCAAA,
(SEQ ID NO: 4)
TGGGGACTTTCCAGTTTCTGGAAAGTCCCCA,
(SEQ ID NO: 5)
CTAGATTTCCCGCG,
or
(SEQ ID NO: 6)
CTAGCGCGGGAAAT.

In various implementations, ODN is 5′-TGGGGACTTTCCAGTTTCTGGAAAGTCCCCA-3′ (SEQ ID NO: 4) which may form a double stranded DNA hairpin structure via intra-dimerization.

In some embodiments, ODN is a double stranded DNA having a sense chain and an anti-sense chain, wherein the sense chain is 5′-CTAGATTTCCCGCG-3′ (SEQ ID NO: 5); and the anti-sense chain is 5′-CTAGCGCGGGAAAT-3′ (SEQ ID NO: 6).

The ODN oligonucleotide may be conjugated to L₁ through the 5′ end of the oligonucleotide (e.g., via the amino modifier C₆).

The compound may be, for example, any compound in Table 2 and formula (Ve) (where the ODN is conjugated through the 5′ end of the oligonucleotide).

TABLE 2
(Ve)

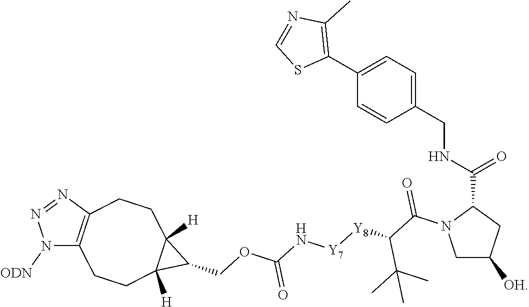

| | Also | | | |
|---|---|---|---|---|
| | Referred | | | |
| | To | | | |
| | Herein | | | SEQ ID |
| Compound | As | —Y₇—Y₈— | ODN | NO: |
| 1 | dNF-κB | —CH₂— | 5′- | 4 |
| | #1 | | TGGGGACTTTCCAGTTTCTGGAAAGTCCCCA- | |
| | | | 3′ | |
| 2 | dNF-κB | —(CH₂)₂— | 5′- | 4 |
| | #2 | | TGGGGACTTTCCAGTTTCTGGAAAGTCCCCA- | |
| | | | 3′ | |
| 3 | dNF-κB | —(CH₂)₃— | 5′- | 4 |
| | #3 | | TGGGGACTTTCCAGTTTCTGGAAAGTCCCCA- | |
| | | | 3′ | |
| 4 | dNF-κB | —(CH₂)₄— | 5′- | 4 |
| | #4 | | TGGGGACTTTCCAGTTTCTGGAAAGTCCCCA- | |
| | | | 3′ | |
| 5 | dNF-κB | —(CH₂)₅— | 5′- | 4 |
| | #5 | | TGGGGACTTTCCAGTTTCTGGAAAGTCCCCA- | |
| | | | 3′ | |
| 6 | dNF-κB | —(CH₂)₆— | 5′- | 4 |
| | #6 | | TGGGGACTTTCCAGTTTCTGGAAAGTCCCCA- | |
| | | | 3′ | |
| 7 | dNF-κB | —(CH₂)₇— | 5′- | 4 |
| | #7 | | TGGGGACTTTCCAGTTTCTGGAAAGTCCCCA- | |
| | | | 3′ | |
| 8 | dNF-κB | —(CH₂)₈— | 5′- | 4 |
| | #8 | | TGGGGACTTTCCAGTTTCTGGAAAGTCCCCA- | |
| | | | 3′ | |
| 9 | dNF-κB | —(CH₂)₉— | 5′- | 4 |
| | #9 | | TGGGGACTTTCCAGTTTCTGGAAAGTCCCCA- | |
| | | | 3′ | |
| 10 | dNF-κB | —(CH₂)₁₀— | 5′- | 4 |
| | #10 | | TGGGGACTTTCCAGTTTCTGGAAAGTCCCCA- | |
| | | | 3′ | |
| 11 | dNF-κB | —CH₂CH₂OCH₂— | 5′- | 4 |
| | #11 | | TGGGGACTTTCCAGTTTCTGGAAAGTCCCCA- | |
| | | | 3′ | |
| 12 | dNF-κB | —(CH₂CH₂O)₂CH₂— | 5′- | 4 |
| | #12 | | TGGGGACTTTCCAGTTTCTGGAAAGTCCCCA- | |
| | | | 3′ | |
| 13 | dNF-κB | —(CH₂CH₂O)₃CH₂— | 5′- | 4 |
| | #13 | | TGGGGACTTTCCAGTTTCTGGAAAGTCCCCA- | |
| | | | 3′ | |
| 14 | dNF-κB | —CH₂CH₂O(CH₂)₂— | 5′- | 4 |
| | #14 | | TGGGGACTTTCCAGTTTCTGGAAAGTCCCCA- | |
| | | | 3′ | |
| 15 | dNF-κB | —(CH₂CH₂O)₂(CH₂)₂— | 5′- | 4 |
| | #15 | | TGGGGACTTTCCAGTTTCTGGAAAGTCCCCA- | |
| | | | 3′ | |
| 16 | dNF-κB | —(CH₂CH₂O)₃(CH₂)₂— | 5′- | 4 |
| | #16 | | TGGGGACTTTCCAGTTTCTGGAAAGTCCCCA- | |
| | | | 3′ | |
| 17 | dNF-κB | —(CH₂CH₂O)₄(CH₂)₂— | 5′- | 4 |
| | #17 | | TGGGGACTTTCCAGTTTCTGGAAAGTCCCCA- | |
| | | | 3′ | |
| 18 | dNF-κB | —(CH₂CH₂O)₅(CH₂)₂— | 5′- | 4 |
| | #18 | | TGGGGACTTTCCAGTTTCTGGAAAGTCCCCA- | |
| | | | 3′ | |
| 19 | dE2F #1 | —CH₂— | 5′-CTAGATTTCCCGCG-3′ | 5 |
| | | | 5′-CTAGCGCGGGAAAT-3′ | 6 |
| 20 | dE2F #2 | —(CH₂)₂— | 5′-CTAGATTTCCCGCG-3′ | 5 |
| | | | 5′-CTAGCGCGGGAAAT-3′ | 6 |
| 21 | dE2F #3 | —(CH₂)₃— | 5′-CTAGATTTCCCGCG-3′ | 5 |
| | | | 5′-CTAGCGCGGGAAAT-3′ | 6 |
| 22 | dE2F #4 | —(CH₂)₄— | 5′-CTAGATTTCCCGCG-3′ | 5 |
| | | | 5′-CTAGCGCGGGAAAT-3′ | 6 |
| 23 | dE2F #5 | —(CH₂)₅— | 5′-CTAGATTTCCCGCG-3′ | 5 |
| | | | 5′-CTAGCGCGGGAAAT-3′ | 6 |
| 24 | dE2F #6 | —(CH₂)₆— | 5′-CTAGATTTCCCGCG-3′ | 5 |
| | | | 5′-CTAGCGCGGGAAAT-3′ | 6 |
| 25 | dE2F #7 | —(CH₂)₇— | 5′-CTAGATTTCCCGCG-3′ | 5 |
| | | | 5′-CTAGCGCGGGAAAT-3′ | 6 |
| 26 | dE2F #8 | —(CH₂)₈— | 5′-CTAGATTTCCCGCG-3′ | 5 |
| | | | 5′-CTAGCGCGGGAAAT-3′ | 6 |
| 27 | dE2F #9 | —(CH₂)₉— | 5′-CTAGATTTCCCGCG-3′ | 5 |
| | | | 5′-CTAGCGCGGGAAAT-3′ | 6 |
| 28 | dE2F #10 | —(CH₂)₁₀— | 5′-CTAGATTTCCCGCG-3′ | 5 |
| | | | 5′-CTAGCGCGGGAAAT-3′ | 6 |
| 29 | dE2F #11 | —CH₂CH₂OCH₂— | 5′-CTAGATTTCCCGCG-3′ | 5 |
| | | | 5′-CTAGCGCGGGAAAT-3′ | 6 |
| 30 | dE2F #12 | —(CH₂CH₂O)₂CH₂— | 5′-CTAGATTTCCCGCG-3′ | 5 |
| | | | 5′-CTAGCGCGGGAAAT-3′ | 6 |
| 31 | dE2F #13 | —(CH₂CH₂O)₃CH₂— | 5′-CTAGATTTCCCGCG-3′ | 5 |
| | | | 5′-CTAGCGCGGGAAAT-3′ | 6 |
| 32 | dE2F #14 | —CH₂CH₂O(CH₂)₂— | 5′-CTAGATTTCCCGCG-3′ | 5 |
| | | | 5′-CTAGCGCGGGAAAT-3′ | 6 |
| 33 | dE2F #15 | —(CH₂CH₂O)₂(CH₂)₂— | 5′-CTAGATTTCCCGCG-3′ | 5 |
| | | | 5′-CTAGCGCGGGAAAT-3′ | 6 |
| 34 | dE2F #16 | —(CH₂CH₂O)₃(CH₂)₂— | 5′-CTAGATTTCCCGCG-3′ | 5 |
| | | | 5′-CTAGCGCGGGAAAT-3′ | 6 |
| 35 | dE2F #17 | —(CH₂CH₂O)₄(CH₂)₂— | 5′-CTAGATTTCCCGCG-3′ | 5 |
| | | | 5′-CTAGCGCGGGAAAT-3′ | 6 |
| 36 | dE2F #18 | —(CH₂CH₂O)₅(CH₂)₂— | 5′-CTAGATTTCCCGCG-3′ | 5 |
| | | | 5′-CTAGCGCGGGAAAT-3′ | 6 |

Pharmaceutical compositions are also provided comprising a compound of the present disclosure (e.g., Compounds having the structure of formula (I)-(V), Compounds 1-36), and one or more pharmaceutically acceptable salts, carriers, or diluents. The present disclosure also provides methods for degrading a transcription factor, the method comprising contacting the transcription factor with a compound of the present disclosure (e.g., Compounds having the structure of formula (I)-(V), Compounds 1-36). Degradation may occur within a cell following contact of the compound with the cell or transfection of the compound into the cell.

Other methods include methods for reducing the proliferation or survival of a neoplastic cell, the method comprising contacting the cell with a compound of the present disclosure (e.g., Compounds having the structure of formula (I)-(V), Compounds 1-36) to induce degradation of transciption factors or subunits thereof in the cell. In some embodiments, the method may involve transfecting the compounds of the present disclosure into the cell. In some embodiments, the neoplastic cell expresses NF-κB or E2F transcription factors. In various implementations, the neoplastic cell is a prostate cancer cell, a myeloma cell, a breast cancer cell, ovarian cancer cell, lymphoma, cervical cancer cell, or lung cancer cell. In some embodiments, the method may comprise transfecting a compound of the present disclosure into a cell.

Transcription factors may also be expressed in various viruses. The present disclosure includes methods for reducing the proliferation or survival of a virion, the method comprising contacting the virus with a compound of the present disclosure (e.g., Compounds having the structure of formula (I)-(V), Compounds 1-36) to induce degradation of transciption factors or subunits thereof in the virion. In some embodiments, the virus expresses NF-κB or E2F transcription factors. In various implementations, the virus is HIV.

Methods for the treatment or prophylaxis of a proliferative disease or virus are also in a subject in need thereof are also provided comprising administering a compound a compound of the present disclosure (e.g., Compounds having the structure of formula (I)-(V), Compounds 1-36), or a pharmaceutical composition containing the compound to the subject. In various embodiments, the proliferative disease is cancer. In some aspects, the proliferative disease is a cancer selected from prostate cancer, multiple myeloma, lymphoma, breast cancer, ovarian cancer, cervical cancer, and lung cancer. In various implementations the viriouns are HIV virions.

Compounds for synthesis of the PROTACs as described herein are also provided. These synthetic intermediates may be a compound having the structure of formula (VI):

Y₂—Y₃—Y₄—Y₅—Y₆—Y₇—Y₈-ULB (VI)

wherein ULB is a ubiquitin ligase binding moiety; and
wherein Y₂ is optionally substituted alkynyl (e.g., C₁-C₁₂ alkynyl), optionally substituted monocyclic or bicyclic cycloalkynyl (e.g., C₃-C₁₂ monocyclic or multicyclic cycloalkynyl, C₃-C₁₂ bicyclic cycloalkynyl), optionally substituted heteroalkynyl (e.g., C₁-C₁₂ heteroalkynyl), or optionally substituted monocyclic or bicyclic heterocycloalkynyl (e.g., C₃-C₁₂ monocyclic heterocycloalkynyl, C₃-C₁₂ bicyclic heterocycloalkynyl);
Y₃-Ys are independently selected from absent, an optionally substituted heteroarylene group (e.g., a five membered heteroarylene, a six membered heteroarylene, a divalent triazole, a divalent imidazole, a divalent pyrrole) optionally fused to an mono or bicyclic cycloalkyl (e.g., C₃-C₁₀ monocyclic cycloalkyl, C₄-C₁₀ bicyclic cycloalkyl such as bicyclo[6.1.0] nonane), —C(O)—, —O—, —OC(O)—, —NR^{a}—, —N(R^{a})C(O)—, —(C(R^{a})(R^{a}))₁₋₁₂, —(C(R^{a})(R^{a})C(R^{a})(R^{a})O)₁₋₁₂—, and —S—S—; and
R^{a} is independently selected at each occurrence from hydrogen and optionally substitued alkyl (e.g., C₁-C₄ alkyl). For example Y₂ may be:

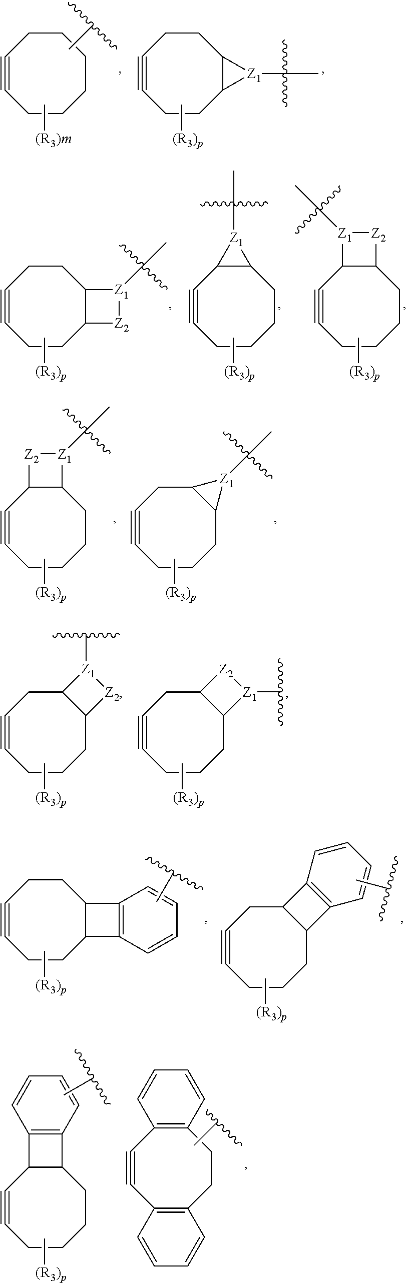

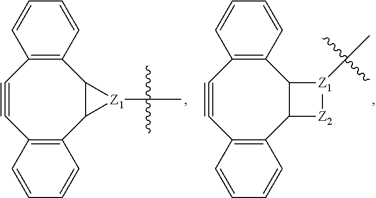

wherein

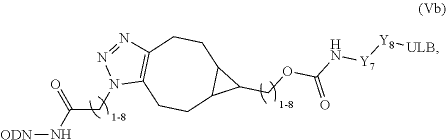

indicates the point of attachment to the neighboring linker group (Y₃);
m is an integer from 0-12 (0, 1, 2, 3, 4, 5, 6, 7, 8, 9, 10, 11, or 12);
p is an integer from 0-10 (0, 1, 2, 3, 4, 5, 6, 7, 8, 9, or 10);
Z₁ is —N— or —CR₃—;
Z₂ is O, C(O), or C(R₃)₂; and
R₃ is independently selected at each occurrence from hydrogen, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.) and wherein any two vicinal R₃ groups of the C₈ ring may together form a five or six membered optionally aromatic ring (e.g., aryl such as phenyl) fused to the C₈ ring.
In some embodiments, the compound may have the structure of formula (VIa):

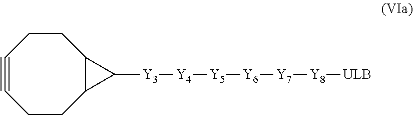

The compound may, for example, have the structure of formula (VIb):

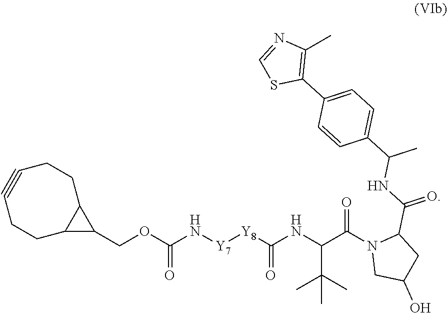

wherein Y₇ and Ys are independently selected from absent (i.e., it is a bond), —(C(R^{a})(R^{a}))₁₋₁₂, and —(C(R^{a})(R^{a})C(R^{a})(R^{a})O)₁₋₁₂—; and
R^{a} is independently selected at each occurrence from hydrogen and optionally substitued alkyl (e.g., C₁-C₄ alkyl). In particular embodiments, the synthetic intermediate is a compound having the structure of formula (VIb) with the Y₇ and Y₈ linkers in Table 1. The synthetic intermediates may have a linker length and be characterized with a click efficiency (e.g., as measured by reaction between a synthetic intermediate of the present disclosure with 10 fold excess of an azide linked ODN group) may be above 10%, above 20% or above 30% or above 40% or above 60% or above 70% or above 80% or above 90% (e.g., from 10%-99%, from 20%-99%, from 30%-99%, 40%-99%, 50%-99%, 60%-99%, 70%-99%, from 10%-99%, from 20%-99%, from 30%-99%, 40%-99%, 50%-99%, 60%-99%, 70%-99%, from 20%-90%, from 30%-90%, 40%-90%, 50%-90%, 60%-90%, 70%-90%, from 20%-90%, from 30%-90%, 40%-90%, 50%-90%, 60%-90%, 70%-90%) efficient.

Methods for forming the PROTACS of the present disclosure a compound (e.g., Compounds having the structure of formula (I)-(V), Compounds 1-36) are provided as well. Typically, these methods comprise reacting (e.g. via an azide-alkyne cycloaddition reaction) a DNA oligomer protein comprising a transcription factor binding motif that binds to a transcription factor or subunit thereof; where said DNA oligomer protein is conjugated to an azide

with a compound having the structure of formula (VI):

Y₂—Y₃—Y₄—Y₅—Y₆—Y₇—Y₈—ULB (VI)

wherein ULB is a ubiquitin ligase binding moiety; and
wherein Y₂ is optionally substituted alkynyl (e.g., C₁-C₁₂ alkynyl), optionally substituted monocyclic or bicyclic cycloalkynyl (e.g., C₃-C₁₂ monocyclic cycloalkynyl, C₃-C₁₂ bicyclic cycloalkynyl), optionally substituted heteroalkynyl (e.g., C₁-C₁₂ heteroalkynyl), or optionally substituted monocyclic or bicyclic heterocycloalkynyl (e.g., C₃-C₁₂ monocyclic heterocycloalkynyl, C₃-C₁₂ bicyclic heterocycloalkynyl);
Y₃-Y₈ are independently selected from absent, an optionally substituted heteroarylene group (e.g., a five membered heteroarylene, a six membered heteroarylene, a divalent triazole, a divalent imidazole, a divalent pyrrole) optionally fused to an mono or bicyclic cycloalkyl (e.g.,

C₃-C₁₀ monocyclic cycloalkyl, C₄-C₁₀ bicyclic cycloalkyl such as bicyclo[6.1.0] nonane), —C(O)—, —O—, —OC(O)—, —NR^{a}—, —N(R^{a})C(O)-, —(C(R^{a})(R^{a}))₁₋₁₂, —(C(R^{a})(R^{a})C(R^{a})(R^{a})O)₁₋₁₂—, and —S—S—; and

R^{a} is independently selected at each occurrence from hydrogen and optionally substitued alkyl (e.g., C₁-C₄ alkyl). The method may further include the step of conjugating an azide to the DNA oligomer protein. For example, the azide can be introduced to an oligonucleotide by attaching an azide-NHS ester functional group to 5′, 3′ or through internal amino modified base or amino linkers such as amino C₆ or amino C₇ for the 3′ end of an oligo. Synthetic steps may occur in physiological conditions such as in phosphate buffered saline (PBS) at from 30° C.-40° C. These synthetic methods be characterized with a click efficiency (e.g., as measured by reaction between a synthetic intermediate of the present disclosure with 10 fold excess of an azide linked ODN group) may be above 10%, above 20% or above 30% or above 40% or above 60% or above 70% or above 80% or above 90% (e.g., from 10%-99%, from 20%-99%, from 30%-99%, 40%-99%, 50%-99%, 60%-99%, 70%-99%, from 10%-99%, from 20%-99%, from 30%-99%, 40%-99%, 50%-99%, 60%-99%, 70%-99%, from 20%-90%, from 30%-90%, 40%-90%, 50%-90%, 60%-90%, 70%-90%, from 20%-90%, from 30%-90%, 40%-90%, 50%-90%, 60%-90%, 70%-90%) efficient.

### BRIEF DESCRIPTION OF FIGURES

FIG. 1 (FIG. 1) is a schematic diagram of the TF-PROTAC strategy. The BCN-modified VHL ligand (VHLL-X-BCN) was incorporated onto azide-modified DNA oligomers (N3-ODNs) through a copper-free strain-promoted azide-alkyne cycloaddition (SPAAC) reaction, forming a TF-PROTAC to recruit VHL E3 ubiquitin ligase and transcription factors (TFs), thus promoting the ubiquitination and subsequently degradation of specific TFs by the 26S proteasome.

FIG. 2 (FIG. 2) provides a schematic of the synthesis of TF-PROTAC. The BCN-modified VHL ligand (VHLL-X-BCN) was incorporated onto azide-modified DNA oligomer (N3-ODN) via a copper-free strain-promoted azide-alkyne cycloaddition (SPAAC) reaction, forming a TF-PROTAC to recruit the VHL E3 ubiquitin ligase as well as the specific transcription factor (TF) to be ubiquitinated by the VHL E3 ubiquitin ligase for subsequent proteasomal degradation.

FIG. 3 (FIGS. 3A-3F) show the verification of the binding effect of Biotin-NF-κB-ODN to p65 in vitro. FIG. 3A is a schematic diagram for the NF-κB-ODN, Biotin-ODN and N3-NF-κB-ODN used in the click reaction. FIG. 3A discloses SEQ ID NOS: 8-9, 1, and 10-12, respectively, in order of appearance. FIG. 3B shows the mass spectrum of N3-NF-κB-ODN. FIG. 3C provides the chemical structure of N3-ODN and Biotin-ODN. FIG. 3D provides the native polyacrylamide gel electrophoresis (PAGE) analysis of N3-NF-κB-ODN before and after annealing. FIG. 3E is a schematic diagram shows the streptavidin-biotin pulldown assay between NF-κB-ODN and p65 transcription factor. FIG. 3F shows the Western blotting of Flag-p65 derived from the streptavidin-biotin pulldown assay with NF-κB-ODN to demonstrate its interaction with p65 transcription factor.

FIG. 4 (FIGS. 4A-4F) provides analyses of the in vitro click reaction that leads to the generation of TF-PROTACs to target NF-κB. FIG. 4A is a schematic diagram for the NF-κB motif and N3-NF-κB-ODN. FIG. 4A discloses SEQ ID NO: 12. FIG. 4B demonstrates that N3-NF-κB is capable of binding with NF-κB subunit p65. FIG. 4C shows that incorporation of VHLL-BCN #1 onto the N3-NF-κB-ODN led to an increase of molecular weight of 660 Da, which could be clearly separated by 20% native polyacrylamide gel electrophoresis (PAGE). FIG. 4D shows the click efficiency between VHLL-BCN #1 and N3-NF-κB-ODN with different molar ratios of reactants. VHLL-BCN #1 and N3-NF-κB-ODN mixture were incubated in PBS at 37 ° C. for indicated time points, followed by being separated by 20% native PAGE. FIG. 4E shows that the TF-PROTAC, dNF-κB #1 competed with Biotin NF-κB for binding to RelA/p65. FIG. 4F shows that dNF-κB#1 induced the VHL E3-PROTAC-p65 ternary complex formation as illustrated by pulldown assay.

FIG. 5 (FIGS. 5A-5C) illustrates the in vitro click reaction to generate the specific TF-PROTACs to target NF-κB. FIG. 5A is a schematic diagram of the SPAAC reaction between VHLL-BCN #1 and N3-NF-κB-ODN, to produce dNF-κB #1. FIG. 5A discloses SEQ ID NOS: 12 and 15, respectively, in order of appearance. FIG. 5B provides the structure of VHLL-BCN #1, azide-group and the rection product (˜ indicates the point of attachment to the ODN group).

FIG. 5C provides native PAGE analysis used resulting in separation of product (dNF-κB #1) and reactants (VHLL-BCN #1 and N3-NF-κB-ODN) of the SPACC reaction therebetween at different molar ratios of reactants.

FIG. 6 (FIGS. 6A-6C) shows how dNF-κB mediates the binding between p65 and the VHL E3 ubiquitin ligase complex in vitro. FIG. 6A is a schematic diagram showing the in vitro binding assay between p65 and the VHL E3 ligase complex. The VHL E3 ligase complexes purified from E. coli were incubated with cell lysis derived from HEK293T cells that ectopically expressed Flag-p65/RelA, with or without the presentence of dNF-κB #1. FIG. 6B shows the Coomassie bright blue staining and western blot analysis of the VHL E3 ligase complex. FIG. 6C is a schematic diagram showing the GST pulldown assay of p65 transcription factor with or without dNF-κB #1.

FIG. 7 (FIGS. 7A-7I) illustrates that dNF-κB promotes the targeted degradation of NF-KB transcription factor in cells. FIG. 7A is a table illustrating the design of eighteen BCN-modified VHL ligand series with different linker between BCN and the VHL ligand (linkers identified herein by # in this Table). FIG. 7B shows that in vitro click of VHLL-X-BCN (X=linker #1-#18) with N3-NF-κB-ODN, which can be separated by native PAGE. FIG. 7C provides the western blot assay for HeLa cells after treated with dNF-κB (#1-#18) for 12 hours. Underlined assays show those linkers (#4, 7, 15, 16, and 18) with the lowest p65 signal. FIG. 7D shows that dNF-κB (#4, 7, 15, 16 and 18) lead to the ubiquitination of p65 in cells. HEK293T cells were transfected with Flag-p65 for 24 hours, and then transfected with indicated dNF-κB for another 12 hours, followed by western blot assay of Ni-NTA pulldown and whole cell lysis (WCL). The proteasome inhibitor MG132 blocked ubiquitinization in these cells (as compared to decreased in intensitites in FIG. 7C). FIG. 7E is a comparison of proteomic changes after treatment with dNF-κB #16 or control in Hela cells. Dotted lines indicate either 50% loss or two-fold increase of the protein level (x-axis) and p=0.01 (y-axis). FIG. 7F shows the pulldown experiments for dNF-κB #15 and #16 with and without proteasome inhibitor MG132. As can be seen, the proteasome inhibitor MG132 blocked the degradation of endogenous p65 by dNF-κB (#15 and 16) in Hela cells. HeLa cells were transfected with indicated dNF-κB and then treated with 10 uM MG132, followed by western blot assay for p65. FIG. 7G shows that dNF-κB (#15 and #16, 10 ug/mL) repressed the proliferation of HeLa cells as measured by cell number. FIGS. 7H-7I show how dNF-κB reduced the tumorigenesis of HeLa cells in a colony formation assay. *: p<0.05.

FIG. 8 (FIGS. 8A-8D) provides analyses for the screening of the in vitro click reaction efficiency between N3-NF-κB-ODN and VHLL-BCN with different linkers (linkers identified in FIG. 7A). FIG. 8A is a schematic diagram of the SPAAC reaction between VHLL-BCN #1 - #18 and N3-NF-κB-ODN, to produce dNF-κB. FIG. 8A discloses SEQ ID NOS: 12 and 16, respectively, in order of appearance. FIGS. 8B-8C show the native PAGE analysis to separate the product of the SPACC reaction between VHLL-BCN #1 - #18 and N3-NF-κB-ODN. FIG. 8D provides the pull-down assays for dNF-κBs (#4, #7, #15, #16 and #18) administration, showing that these TF-PROTACS recruited the binding between the VHL E3 ubiquitin ligase and p65 in vitro.

FIG. 9 (FIG. 9) shows the STRING network involving RelA/65 and the proteins that were down-regulated in Hela cells treated with NF-κB degrader (dNF-κB #16) compared to vehicle-treated cells. All but STX6 may be possibly used for regulation of gene expression within the STRING network. (STRING: https://string-db.org/)

FIG. 10 (FIGS. 10A-10E) show that dNF-κB degrades p65 in a VHL E3 ubiquitin ligase-dependent manner. FIG. 10A illustrates that VHLL-BCN (#15 - #18, 1 μM) were incapable of degrading p65 in HeLa cells. FIG. 10B shows that co-treatment with a VHL ligand (VH-032, 10 μM) blocked the degradation of p65 induced by dNF-κB #16 in Hela cells. FIG. 10C shows that dNF-κB #16 (10 μg/mL) was incapable of degrading p65 in Hela cells after depletion of endogenous VHL using CRISPR-Cas9. FIG. 10D provides the chemical structures of VHLL-BCN #16 and its diastereoisomer VHLL-BCN #16-NC, which is a negative control of VHLL-BCN #16 (and used to form a negative control of dNFOKB #16). FIG. 10E shows that dNF-κB #16-NC (10 μg/mL), the SPAAC reaction product from VHLL-BCN #16-NC and N3-NF-κB-ODN, was incapable of degrading p65 in Hela cells.

FIG. 11 (FIGS. 11A-11D) describe the SPAAC reaction between N3-E2F-ODN and VHLL-X-BCN to produce dE2F and analysis thereof. FIG. 11A is a schematic diagram showing the structure of E2F motif, E2F-ODN, Biotin-E2F-ODN and N3-E2F-ODN. FIG. 11A discloses

SEQ ID NOS: 13, 6, 5-6, 14, and 6, respectively, in order of appearance. FIG. 11B are the mass spectra of N3-E2F-sense and E2F-antisense oligomers. FIGS. 11C-11D show the western blotting of HA-E2F1 derived from the streptavidin-biotin pulldown assay of E2F-ODN with HA-E2F1 transcription factor.

FIG. 12 (FIGS. 12A-12E). Design and in vitro click of dE2F. FIG. 12A is a schematic diagram for the E2F motif and the sequence of N3-E2F-ODN. FIG. 12A discloses SEQ ID NOS: 14 and 6, respectively, in order of appearance. FIG. 12B shows pulldown of heterodimer in relation to annealing and formation of double strain N3-E2F-ODN. FIG. 12C shows that N3-E2F-ODN is capable of binding with E2F1 transcription factor. FIG. 12D show the in vitro click of VHLL-BCN (#1˜#18) with N3-E2F-ODN, which can be separated by 20% native PAGE. FIG. 12E provides the western blot assay results for HeLa cells after treated with dE2F (#1˜#18) for 12 hours.

FIG. 13 (FIGS. 13A-13E) shows the design of the dE2F TF-PROTACs to achieve targeted degradation of E2F1. FIG. 13A is a schematic diagram showsing the SPAAC reaction between N3-E2F-ODN (SEQ ID NOS: 14 and 6) and VHLL-X-BCN to produce dE2F (SEQ ID NOS: 17 and 6). FIG. 13B illustrates that VHLL-BCN (#15 - #18, 1 M) were incapable of degrading E2F1 in Hela cells. FIG. 13C shows that dE2Fs (#15, #16 and #17) recruited the binding between the VHL E3 ubiquitin ligase and E2F1 transcription factor in vitro. FIG. 13D provides evidence that co-treatment with a VHL ligand (VH-032, 10 μM) blocked the degradation of E2F1 induced by dE2F #16 (25 μg/mL) in Hela cells. FIG. 13E illustrates that dE2F #16 (25 μg/mL) was incapable of degrading E2F1 in HeLa cells after depletion of endogenous WHI, using CRISPR-Cas9.

FIG. 14 (FIGS. 14A-14F) shows that dE2F promotes the degradation of E2F1 transcription factor in HeLa cells. FIG. 14A provides the dose-dependent assay of dE2F1 #16 and #17 in degradation of E2F1 in HeLa cells. HeLa cells were transfected with dE2F1 #16 and #17 for 12 hours, followed by western blot assay of E2F1. (FIG. 14B shows that the proteasome inhibitor MG132 blocked the degradation of endogenous E2F1 by dE2F1 in cells (#16 and 17). HeLa cells were transfected with indicated dE2F1 and then treated with 30 μM MG132, followed by western blot assay for E2F1. FIG. 14C illustrates that dE2F1 led to ubiquitination of E2F1 in cells. HeLa cells were transfected with HA-E2F1 for 24 hours, then treated with dE2F1 (#16 and 17) for 12 hours, followed by western blot assay of Ni-NTA pulldown and WCL. FIG. 14D provides that dE2F1 repressed the proliferation of HeLa cells after the treatment of dE2F1 (#16 and 17) in measurements of cell numbers at specific time points following administration. FIGS. 14E-14F show that dE2F1 reduced the tumorigenesis of HeLa cells after the treatment of dE2F1 (#16 and 17) in a colony formation assay. ** , ***: p<0.01, p<0.001, respectively.

### DETAILED DESCRIPTION

Detailed embodiments of the present disclosure are disclosed herein; however, it is to be understood that the disclosed embodiments are merely illustrative and may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. Moreover, all values that fall within these ranges, as well as the upper or lower limits of a range of values, are also contemplated by the present application. For example, the exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that sum of all weight percentages does not exceed 100% unless otherwise specified.

By “consist essentially” it is meant that the ingredients include only the listed components along with the normal impurities present in commercial materials and with any other additives present at levels which do not affect operation, for instance at levels less than 5% by weight or less than 1% or even 0.5% by weight.

Typically, alkyl groups described herein refer to a branched or straight-chain monovalent saturated aliphatic hydrocarbon radical of 1-30 carbon atoms (e.g., 1-16 carbon atoms, 6-20 carbon atoms, 8-16 carbon atoms, or 4-18 carbon atoms, 4-12 carbon atoms, 1-4 carbon atoms, 1-8 carbon atoms) which are optionally substituted. In some embodiments, the alkyl group may be substituted with 1, 2, 3, or 4 substituent groups as defined herein. Alkyl groups may have from 1-26 carbon atoms. In other embodiments, alkyl groups will have from 6-18 or from 1-8 or from 1-6 or from 1-4 or from 1-3 carbon atoms, including for example, embodiments having one, two, three, four, five, six, seven, eight, nine, or ten carbon atoms. Any alkyl group may be substituted or unsubstituted. Examples of alkyl groups include methyl, ethyl, propyl, butyl, pentyl, hexyl, heptyl, octyl, nonyl, decyl, undecyl, and dodecyl groups. Heteroalkyl groups may refer to branched or straight-chain monovalent saturated aliphatic hydrocarbon radicals with one or more heteroatoms (e.g., N, O, S, etc.) in the carbon chain. Heteroalkyl groups may have 1-30 carbon atoms (e.g., 1-16 carbon atoms, 6-20 carbon atoms, 8-16 carbon atoms, or 4-18 carbon atoms, 4-12 carbon atoms, etc.). In some embodiments, the heteroalkyl group may be substituted with 1, 2, 3, or 4 substituent groups as defined herein. Heteroalkyl groups may have from 1-26 carbon atoms. In other embodiments, heteroalkyl groups will have from 6-18 or from 1-8 or from 1-6 or from 1-4 or from 1-3 carbon atoms, including for example, embodiments having one, two, three, four, five, six, seven, eight, nine, or ten carbon atoms. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O-; and “alkoyl” which, as used herein, refers to alkyl-CO-. Alkoxy substituent groups or alkoxy-containing substituent groups may be substituted by, for example, one or more alkyl groups. Alkenyl groups include alkyl groups having a double bond between any two carbon atoms. Alkynyl groups include alkyl groups having a triple bond between carbon atoms.

Aryl groups may be aromatic mono- or polycyclic radicals of 6 to 12 carbon atoms having at least one aromatic ring. Aryl groups may be optionally substituted. Examples of such groups include, but are not limited to, phenyl, naphthyl, 1,2,3,4-tetrahydronaphthalyl, 1,2-dihydronaphthalyl, indanyl, and 1H-indenyl. Aryl groups as used herein may be substituted or unsubstitued. Typically, heteroaryls include mono- or polycyclic radical of 5 to 12 atoms having at least one aromatic ring containing one, two, or three ring heteroatoms selected from N, O, and S, with the remaining ring atoms being C. One or more ring carbon atoms of the heteroaryl group may be replaced with a carbonyl group. Examples of heteroaryl groups are pyridyl, triazolyl, benzooxazolyl, benzoimidazolyl, and benzothiazolyl.

Linking groups, when present, may be referred to as divalent hydrocarbon groups (e.g., alkylene, heteroalkylene, alkynylene, alkeneylene, heteroalkynylene, heteroalkeneylene, arylene, heteroarylene), where each radical position is the point of attachment to the remaining portions of the compound. Alkylene groups may refer to a straight or branched chain divalent hydrocarbon radical having from one to ten carbon atoms, optionally substituted with substituents, for example, selected from the group which includes lower alkyl (e.g., C₁-C₄, etc.), lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen and lower perfluoroalkyl, multiple degrees of substitution being allowed. Examples of alkylene as used herein include, but are not limited to, methylene, ethylene, n-propylene, n-butylene, and the like.

Alkylene groups may be saturated or unsaturated. Heteroalkylene groups may be alkylene groups comprising one or more heteroatoms (e.g., N, S, O) in the carbon chain. Cycloalkylene groups may be divalent hydrocarbons comprising one or more saturated or unsaturated cycloalkyl groups. The two points of attachment on cycloalkylene groups may be at two points in the ring, for example, at vicinal positions or at geminal positions. Cycloalkylene groups may be divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic unsaturated ring systems that respectively include at least one double bond and at least one triple bond. Cycloalkylene, Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms.

It will be understood that any divalent linking moiety with multiple points of attachment (each typically indicated with “—”) may be attached to the specified moieties in either direction to the extent permitted by valency, unless otherwise indicated (e.g., by indicating the attachments on each side). For example, a linking moiety having the structure —Y₁—Y₂— may be used to link two portions of a compound in the —Y₁—Y₂— orientation or in the —Y₂—Y₁— orientation. However, ODN—Y₁—Y₂— ULB does not include ODN—Y₂—Y₁— ULB unless otherwise indicated.

The ring systems of the saturated, partially saturated, or unsaturated cycloalkyl, aryl, heterocylcoalkyl, heteroaryl, cycloalkylene, cycloalkenylene cycloalkynylene, heterocycloalkylene, heterocycloalkenylene and heterocycloalkynylene groups may be composed of one ring, two rings, or three or more rings (e.g., from 1 to 5 rings) which may be joined together in a fused, bridged or spiro-connected fashion, each of which may be optionally substituted. Heteocyclo and Heterocyclene groups may be monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur. Aryl and Arylene groups may be monocyclic or polycyclic, in certain embodiments monocyclic, aromatic group, in some embodiments having from 5 to 20 carbon atoms and at least one aromatic ring, in another embodiment 5 to 12 carbons. Arylene groups include, but are not limited to, 1,2-, 1,3- and 1,4-phenylene. Heteroarylene groups are typically divalent monocyclic or multicyclic aromatic ring systems, in one embodiment of 5 to 15 atoms in the ring(s), where one or more, in certain embodiments 1 to 3, of the atoms in the ring system is a heteroatom, that is, an element other than carbon, including but not limited to, nitrogen, oxygen or sulfur. In some embodiments, heteroarylene or heterocyclene groups (particularly in linking moieties) are divalent optionally substituted multicyclic heteroaryl or heterocyclyl fused with a monocyclic or multicyclic (e.g., bicyclic, tricyclic) optionally saturated optionally substituted cycloalkyl, wherein one point of attachment to the rest of the compound is on the heteroaryl moiety and the other point of attachment to the rest of the compound is on the cycloalkyl moiety. For example, the multicyclic heteroarylene or heterocyclene groups (particularly as a linking moiety such as Y₂ or Y₃ as a a strained click chemistry reaction product —L_{SCC}) may have have the structure:

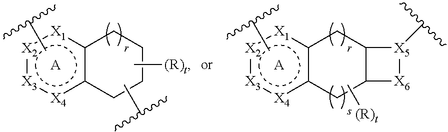

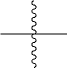

wherein wherein each indicates a point of attachment to the compound;
ring “A” is optionally aromatic five or six membered ring;
r and s are independently 0, 1, 2, 3, 4, or 5;
t is 0, 1, 2, 3, 4, 5, 6, 7, 8, 9, 10, 11, 12, 13, or 14;
X₁-X₄ are independently selected at each occurrence from absent, CR, C(R)₂, N, NR, O, S or SR;

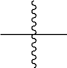

one of X₁-X₄ comprises a (e.g., in place of an R group); and at least one of X₁-X₄ is not CR or C(R)₂;

### X₅ is CR or N;

X₆ is absent, C(R)₂, NR, or O;
R is independently selected at each occurrence from hydrogen, halogen, —OR^{a}, —N(R^{a})(R^{a}), alkyl (e.g., C₁-C₇ alkyl, C₁-C₃ alkyl, etc.), or alkoxy (e.g., C₁-C₇ alkoxy, C₁-C₃ alkoxy, etc.) wherein any two vicinal R groups may together form a fused ring (e.g., five membered fused ring, six membered fused ring, five membered fused aromatic ring, six membered fused aromatic ring); and
R^{a} is independently selected at each occurrence from hydrogen, or alkyl (e.g., C₁-C₇ alkyl, C₁-C₄ alkyl, etc.).

The term “substituent” refers to a group “substituted” on, e.g., an alkyl, at any atom of that group, replacing one or more hydrogen atoms therein. In some aspects, the substituent(s) on a group are independently any one single, or any combination of two or more of the permissible atoms or groups of atoms delineated for that substituent. In another aspect, a substituent may itself be substituted with any one of the substituents described herein.

A substituted hydrocarbon group may have as a substituent one or more hydrocarbon radicals, substituted hydrocarbon radicals, or may comprise one or more heteroatoms. Examples of substituted hydrocarbon radicals include, without limitation, heterocycles, such as heteroaryls. Unless otherwise specified, a hydrocarbon substituted with one or more heteroatoms will comprise from 1-20 heteroatoms. In other embodiments, a hydrocarbon substituted with one or more heteroatoms will comprise from 1-12 or from 1-8 or from 1-6 or from 1-4 or from 1-3 or from 1-2 heteroatoms. Examples of heteroatoms include, but are not limited to, oxygen, nitrogen, sulfur, phosphorous, halogen (e.g., F, Cl, Br, I, etc.), boron, silicon, etc. In some embodiments, heteroatoms will be selected from the group consisting of oxygen, nitrogen, sulfur, phosphorous, and halogen (e.g., F, Cl, Br, I, etc.). In some embodiments, a heteroatom or group may substitute a carbon. In some embodiments, a heteroatom or group may substitute a hydrogen. In some embodiments, a substituted hydrocarbon may comprise one or more heteroatoms in the backbone or chain of the molecule (e.g., interposed between two carbon atoms, as in “oxa”). In some embodiments, a substituted hydrocarbon may comprise one or more heteroatoms pendant from the backbone or chain of the molecule (e.g., covalently bound to a carbon atom in the chain or backbone, as in “oxo”).

In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent (e.g., a hydrocarbon), the group may be referred to as “R-substituted.” Where a moiety is R-substituted, the moiety is substituted with at least one R substituent and each R substituent is optionally different.

Unless otherwise noted, all groups described herein (e.g., alkyl, cycloalkyl, heteroalkyl, heterocycloalkyl, aryl, heteroaryl, alkylene, heteroalkylene, cylcoalkylene, arylene, heteroaryelene, heterocycloalkylene) may optionally contain one or more common substituents, to the extent permitted by valency. Common substituents include halogen (e.g., F, Cl, etc.), C₁-12 straight chain or branched chain alkyl, C₂-12 alkenyl, C₂-12 alkynyl, C₃-12 cycloalkyl, C₆-12 aryl, C₃-12 heteroaryl, C₃-12 heterocyclyl, C₁-12 alkylsulfonyl, nitro, cyano, —COOR, —C(O)NRR′, —OR, —SR, —NRR′, and oxo, such as mono- or di- or tri-substitutions with moieties such as halogen, fluoroalkyl, perfluoroalkyl, perfluroalkoxy, trifluoromethoxy, chlorine, bromine, fluorine, methyl, methoxy, pyridyl, furyl, triazyl, piperazinyl, pyrazoyl, imidazoyl, and the like, each optionally containing one or more heteroatoms such as halo, N, O, S, and P. R and R′ are independently hydrogen, C₁-12 alkyl, C₁-12 haloalkyl, C₂-12 alkenyl, C₂-12 alkynyl, C₃-12 cycloalkyl, C₄-24 cycloalkylalkyl, C₆-12 aryl, C₇-24 aralkyl, C₃-12 heterocyclyl, C₃-24 heterocyclylalkyl, C₃-12 heteroaryl, or C₄-24 heteroarylalkyl. Further, as used herein, the phrase optionally substituted indicates the designated hydrocarbon group may be unsubstituted (e.g., substituted with H) or substituted with, for example one or more independently chosen common substituents. Typically, substituted hydrocarbons are hydrocarbons with a hydrogen atom removed and replaced by a substituent (e.g., a common substituent). It is understood by one of ordinary skill in the chemistry art that substitution at a given atom is limited by valency. The use of a substituent (radical) prefix names such as alkyl without the modifier optionally substituted or substituted is understood to mean that the particular substituent is optionally substittued. Additionally, the use of haloalkyl without the modifier optionally substituted or substituted is still understood to mean an alkyl group, in which at least one hydrogen atom is replaced by halo. Compounds provided herein (e.g., Compounds having the structure of formula (I)-(V),

Compounds 1-36) can have one or more asymmetric carbon atoms and can exist in the form of optically pure enantiomers, mixtures of enantiomers such as racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the conFIGuration of substituents around one or more chiral carbon atoms. Enantiomers of a compound can be prepared, for example, by separating an enantiomer from a racemate using one or more well-known techniques and methods, such as chiral chromatography and separation methods based thereon. The appropriate technique and/or method for separating an enantiomer of a compound described herein from a racemic mixture can be readily determined by those of skill in the art. “Racemate” or “racemic mixture” means a mixture containing two enantiomers, wherein such mixtures exhibit no optical activity; i.e., they do not rotate the plane of polarized light. “Geometric isomer” means isomers that differ in the orientation of substituent atoms (e.g., to a carbon-carbon double bond, to a cycloalkyl ring, to a bridged bicyclic system, etc.). Atoms (other than H) on each side of a carbon- carbon double bond may be in an E (substituents are on opposite sides of the carbon- carbon double bond) or Z (substituents are oriented on the same side) conFIGuration. “R,” “S,” “S*,” “R*,”“E,” “Z,” “cis,” and “trans,” indicate conFIGurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds disclosed herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99%, or 99.9%) by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The disclosure embraces all of these forms.

It will be understood that the description of compounds herein is limited by principles of chemical bonding known to those skilled in the art. Accordingly, where a group may be substituted by one or more of a number of substituents, such substitutions are selected so as to comply with principles of chemical bonding with regard to valencies, etc., and to give compounds which are not inherently unstable. For example, any carbon atom will be bonded to two, three, or four other atoms, consistent with the four valence electrons of carbon. Additionally, when a structure has less than the required number of functional groups indicated, those carbon atoms without an indicated functional group are bonded to the requisite number of hydrogen atoms to satisfy the valency of that carbon.

The term “pharmaceutical composition,” as used herein, represents a composition containing a compound described herein (e.g., Compounds having the structure of formula (I)-(V), Compounds 1-36) formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, or syrup); for topical administration (e.g., as a cream, gel, lotion, or ointment); for intravenous administration (e.g., as a sterile solution free of particulate emboli and in a solvent system suitable for intravenous use); or in any other formulation described herein (see below). The pharmaceutical composition may comprise, for example, from 0.1% to 25% of the compounds of the present disclosure by weight of the composition.

Useful pharmaceutical carriers for the preparation of the compositions hereof, can be solids, liquids, or gases. Thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, and aerosols. The carrier can be selected from the various oils including those of petroleum, animal, vegetable or synthetic origin, e.g., peanut oil, soybean oil, mineral oil, and sesame oil. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution and rendering the solution sterile. Suitable pharmaceutical excipients include starch, cellulose, chitosan, talc, glucose, lactose, gelatin, malt, rice, flour, chalk, silica, magnesium stearate, sodium stearate, glycerol monostearate, sodium chloride, dried skim milk, glycerol, propylene glycol, water, and ethanol. The compositions may be subjected to conventional pharmaceutical additives such as preservatives, stabilizing agents, wetting or emulsifying agents, salts for adjusting osmotic pressure, and buffers. Suitable pharmaceutical carriers and their formulation are described in Remington's Pharmaceutical Sciences by E. W. Martin, which is hereby incorporated by reference in its entirety. Such compositions will, in any event, contain an effective amount of the active compound together with a suitable carrier so as to prepare the proper dosage form for administration to the recipient.

As used herein, the term “pharmaceutically acceptable salt” refers to salts of any of the compounds described herein that within the scope of sound medical judgment, are suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P.H . Stahl and C. G. Wermuth), Wiley-V C H, 2008, each of which are hereby incorporated by reference in their entirety. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecylsulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine.

Transcription factors typically refer to a DNA-binding protein that regulates transcription of DNA into RNA, for example, by activation or repression of transcription. Some transcription factors effect regulation of transcription alone, while others act in concert with other proteins. Some transcription factors can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules. Examples of transcription factors include, but are not limited to, Sp1, NF1, CCAAT, GATA, HNF, PIT-1, MyoD, Myf5, Hox, Winged Helix, SREBP, p53, CREB, AP-1, Mef2, STAT, R-SMAD, NF-κB, Notch, TUBBY, and NFAT. Transcription factors generally bind DNA in a sequence-specific manner and either activate or repress transcription initiation.

At least three types of separate domains have been identified within transcription factors. One is essential for sequence-specific DNA recognition, one for the activation/repression of transcriptional initiation, and one for the formation of protein-protein interactions (such as dimerization). Studies indicate that many plant transcription factors can be grouped into distinct classes based on their conserved DNA binding domains (Katagiri F and Chua N H, 1992, Trends Genet. 8:22-27; Menkens A E, Schindler U and Cashmore A R, 1995, Trends in Biochem Sci. 13:506-510; Martin C and Paz-Ares J, 1997, Trends Genet. 13:67-73). Each member of these families interacts and binds with distinct DNA sequence motifs that are often found in multiple gene promoters controlled by different regulatory signals.

As used herein, the term “subject” refers to any organism to which a composition in accordance with the disclosure may be administered, e.g., for experimental, diagnostic, prophylactic, and/or therapeutic purposes. In most embodiments, the subject is a human. Other subjects may include mammals such as mice, rats, rabbits, cats, dogs, non-human primates. The subject may be domesticated animals (e.g., cows, calves, sheep, goat, lambs, horses, poultry, foals, pigs, piglets, etc.), or animals in the family Muridae (e.g., rats, mice, etc.), or animals in the family Felidae. A subject may seek or be in need of treatment, require treatment, be receiving treatment, may be receiving treatment in the future, or a human or animal that is under care by a trained professional for a particular disease or condition (e.g., cancer, etc.).

Physiological conditions typically refer to a set of conditions including temperature, salt concentration, pH that mimic those conditions of a living subject. A host of physiologically relevant conditions for use in in vitro assays and chemical syntheses have been established. Generally, a physiological buffer contains a physiological concentration of salt and at adjusted to a neutral pH ranging from about 6.5 to about 7.8, and preferably from about 7.0 to about 7.5. A variety of physiological buffers may be used to produce physiological conditions in, for example, reaction medium, including phosphate buffered saline (PBS). Physiologically relevant temperature ranges from 25° C. to 40° C. or from 30° C. to 38° C.

Typically, a proliferative disease refers to the physiological condition in a subject characterized by unregulated cell growth such as cancer. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small cell lung cancer, non-small cell lung cancer (“NSCLC”), vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, as well as head and neck cancer. In yet other embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma, myeloproliferative diseases, large B cell lymphoma, and B cell Lymphoma.

The term “unit dosage form” refers to a physically discrete unit suitable as a unitary dosage for human subjects and other mammals, each unit containing a predetermined quantity of active material (e.g., a compound of the present disclosure) calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, gel cap, and syrup.

The term “effective amount” or “therapeutically effective amount” of an agent, as used herein (e.g., Compounds having the structure of formula (I)-(V), Compounds 1-36), is that amount sufficient to effect beneficial or desired results, such as clinical results, and, as such, an “effective amount” depends upon the context in which it is being applied. In one embodiment, an effective amount is the amount of an irradiated compound described herein sufficient to affect the degradation of a protein of interest. In another embodiment, in the context of administering an agent that is an anticancer agent, an effective amount of an agent is, for example, an amount sufficient to achieve alleviation or amelioration or prevention or prophylaxis of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition (e.g., cancer, etc.); and remission (whether partial or total), whether detectable or undetectable, as compared to the response obtained without administration of the agent. In some embodiments, the effective amount assumes that more than 50% of the compounds administered release the photolabile group under irradiation conditions (e.g., more than 60%, more than 70%, more than 80%, more than 90%, more than 95%, more than 99%, 100%, etc.) to achieve the active compound capable of degrading a protein of interest.

The functioning of the PROTAC is typically dependent on the binding of the ODN moiety to the protein of interest to bring the ULB moiety proximal to the TF. In certain embodiments, the ODN (or PROTAC comprising the ODN) may have an affinity for its Transcription Factor or binding motif thereof (K_{d}) of less than 1 mM (e.g. from 500 μM to 1 mM, etc.) or less than 500 μM (e.g., less than 450 μM, less than 400 μM, less than 350 μM, less than 300 μM, less than 250 μM, less than 200 μM, less than 150 μM, less than 100 μM, less than 100 μM, less than 50 μM, less than 10 μM, less than 1 μM, less than 500 nM, less than 100 nM, less than 50 nM, less than 10 nM, less than 1 nM, etc.).

The compounds, methods and compositions described herein include the use of N-oxides (if appropriate), crystalline forms (also known as polymorphs), solvates, amorphous phases, and/or pharmaceutically acceptable salts of compounds having the structure of any compound of the disclosure, as well as metabolites and active metabolites of these compounds having the same type of activity. Solvates include water, ether (e.g., tetrahydrofuran, methyl tert-butyl ether, etc.) or alcohol (e.g., ethanol, etc.) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compounds of the disclosure may exist as tautomers or mixtures of tautomers, racemates, mixtures of racemates, stereoisomers, mixtures of stereoisomers, or combinations thereof. All tautomers are included within the scope of the compounds presented herein.

In certain embodiments, compounds described herein may be converted into an active PROTAC in vivo following endocytosis (and, particularly, targeting group assited endocytosis), and, in particular, endocytosis for disease causing cells such as proliferative cells (e.g., cancer cells). In some embodiments, the PROTAC is caged with a photolabile group which may be removed optically to begin ubiquitin recruitment such as those described in WO2021016521, which is hereby incorporated by reference in its entirety, and particularly in relation to glutaramide based ULB caging. In certain embodiments, upon in vivo administration, the targeting group caged PROTAC is chemically converted to the biologically, pharmaceutically or therapeutically active form of the compound. For example, the compound of the present disclosure may have the structure of

ODN—L₁—ULB—PLG,

or

ODN—L₁—ULB—L₂—TG;

wherein PLG is a nitrophenyl based photolabile group (e.g., nitrobenzyl, ortho-nitrobenzyl, nitroveratryloxycabonyl such as 6-nitroveratryloxycarbonyl);
L₂ is a linking moiety; and
TG is a targeting group that preferentially binds to a protein with increased expression in a neoplastic cell as compared to an otherwise identical healthy cell. In various implementations. Suitable L₂, PLG, and TG groups may be found for example in WO2021016521, U.S. App. No. 63/144,442 and U.S. App. No. 63/186,005, each of which are hereby incorporated by reference in their entirety and particularly in relation to PLG, TG, and L₂ conjugated to ULB. If caged, the compounds of the present disclosure may be converted into compound capable of binding to the transcription factor of interest, where cleavage of the targeting group induces ubiquitin recruitment within the cell following interaction with endogenous hydrolases. In other embodiments, the compounds of the present disclosure may be enzymatically metabolized by one or more steps or processes to the biologically, pharmaceutically or therapeutically active form of the compound during and/or following endocytosis.

The disclosure includes a pharmaceutical composition comprising at least one compound described herein (e.g., Compounds having the structure of formula (I)-(V), Compounds 1-36) and at least one pharmaceutically acceptable carrier, diluent, or diluent. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

The pharmaceutical composition may comprise a compound disclosed herein (e.g., Compounds having the structure of formula (I)-(V), Compounds 1-36) and one or more pharmaceutically acceptable salts, carriers, or diluents. In specific embodiments, the compound is formulated as a topical composition (e.g., ointment, gel, etc.). In some embodiments, the composition comprises from 0.1%-90% (e.g., 0.1%-50%, 0.1%-20%, 0.1%-10%, etc.) of the compound by weight of the composition.

The pharmaceutical composition may be formulated to allow for or enhance transfection of the compounds of the present disclosure (e.g., Compounds having the structure of formula (I)-(V), Compounds 1-36) into a cell. Transfection may occur, for example, in vivo, ex vivo, or in vitro. The compounds of the present disclosure can be introduced by suitable transfection mechanisms such as electroporation, lipofection, particle gun acceleration. In some example the method is a chemical method (e.g., calcium-phosphate transfection), physical method (e.g., electroporation, microinjection, particle bombardment), fusion (e.g., liposomes), or receptor-mediated endocytosis.

In some embodiments, a pharmaceutical composition may comprise one or more lipids with the compounds of the presente disclosure selected from cationic lipids, ionizable lipids, anionic lipids, sterols, pegylated lipids, and any combination of the foregoing. In some embodiments, the pharmaceutical composition containing a translatable compound comprises a cationic lipid, a phospholipid, cholesterol, and a pegylated lipid.

In certain embodiments, a pharmaceutical composition can be substantially free of liposomes. In some embodiments, the pharmaceutical composition comprises liposomes. In some embodiments, the pharmaceutical composition comprises lyophilized liposomes. In further embodiments, a pharmaceutical composition can include nanoparticles. Lipid-based formulations have been increasingly recognized as one of the most promising delivery systems due to their biocompatibility with certain DNA and RNA and their ease of large-scale production. Cationic lipids, for example, have been widely studied as synthetic materials for compound. After mixing together, nucleic acids may be condensed by cationic lipids to form lipid/nucleic acid complexes known as lipoplexes. These lipid complexes may be able to protect compounds of the present disclosure from the action of nucleases and to deliver it into cells by interacting with the negatively charged cell membrane. Lipoplexes can be prepared by directly mixing positively charged lipids at physiological pH with negatively charged nucleic acids.

Conventional liposomes typically consist of a lipid bilayer that can be composed of cationic, anionic, or neutral (phospho)lipids and cholesterol, which encloses an aqueous core. Both the lipid bilayer and the aqueous space can incorporate hydrophobic or hydrophilic compounds, respectively. Liposome characteristics and behaviour in vivo can be modified by addition of a hydrophilic polymer coating, e.g. polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains (Front Pharmacol. 2015 Dec. 1; 6:286, which is hereby incorporated by reference in its entirety).

Pharmaceutical compostions of the present disclosure comprising liposomes may be colloidal lipid-based and surfactant-based delivery systems composed of a phospholipid bilayer surrounding an aqueous compartment. These liposomes may be present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm.

Cationic liposomes have been traditionally the most commonly used non-viral delivery systems for oligonucleotides, including plasmid DNA, antisense oligos, and siRNA/small hairpin R A-shRNA). Cationic lipids, such as DOTAP (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high transfection efficiency. Furthermore, neutral lipid-based nanoliposomes for compound delivery as e.g. neutral 1,2-dioleoyl-sn-glycero-3-phosphatidylcholine (DOPC)-based nanoliposomes may be well suited for delivery of the compounds of the present disclosure (Adv Drug Deliv Rev. 2014 February; 66: 110-116, which is hereby incorporated by reference in its entirety).

According to some embodiments, the compounds described herein (e.g., Compounds having the structure of formula (I)-(V), Compounds 1-36) are lipid formulated. The lipid formulation is preferably selected from, but not limited to, liposomes, lipoplexes, copolymers, such as PLGA, and lipid nanoparticles. Lipid nanoparticles may include a cationic lipid suitable for forming a lipid nanoparticle. The cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-dilinoleyloxy-N,N-dimethylaminopropane (DLinDMA), 1,2-dilinolenyloxy-N,N-dimethylaminopropane (DLenDMA), 1,2-di-y-linolenyloxy-N,N-dimethylaminopropane (γ-DLenDMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.CI), 1,2-dilinoleoyl-3-trimethylaminopropane chloride salt (DLin-TAP.CI), 1,2-dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA), (3aR,5s,6aS)-N,N-dimethyl-2,2-di((9Z,12Z)-octadeca-9,12-dienyl)tetrahydro—3aH-cyclopenta[d][1,3]dioxol-5-amine, (6Z,9Z,28Z,31Z)-heptatriaconta-6,9,28,31-tetraen-19-yl4-(dimethylamino)butanoate (MC₃), 1, 1′-(2-(4-(2-((2-(bis(2-hydroxydodecyl)amino)ethyl)(2-hydroxydodecyl)ami- no)ethyl)piperazin-1- yl)ethylazanediyl)didodecan-2-ol (C₁₂-200), 2,2-dilinoleyl-4-(2-dimethylaminoethyl)-[1,3]-dioxolane (DLin-K-C₂-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C₃-DMA), 3-((6Z,9Z,28Z,31Z)-heptatriaconta-6,9,28,31-tetraen-19-yloxy)-N,N-dimethy- lpropan-1-amine (MC₃ Ether), 4-((6Z,9Z,28Z,31 Z)-heptatriaconta-6,9,28,31-tetraen-19-yloxy)-N,N-dimethylbutan-1-amine (MC₄ Ether), or any combination of any of the foregoing. Other cationic lipids include, but are not limited to, N,N-distearyl-N,N-dimethylammonium bromide (DDAB), 3P-(N-(N′,N′-dimethylaminoethane)-carbamoyl)cholesterol (DC-Choi), N-(1-(2,3-dioleyloxy)propyl)-N-2-(sperminecarboxamido)ethyl)-N,N-dimethyl- ammoniumtrifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-y1)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al, PNAS, 107.5 (2010): 1864-69, each of which are hereby incorporated by reference in their entirety. Other suitable amino lipids useful in the compositions of the present disclosure include those having alternative fatty acid groups and other dialkylamino groups, including those, in which the alkyl substituents are different (e.g., N-ethyl-N-methylamino-, and N-propyl-N-ethylamino-). In general, amino lipids having less saturated acyl chains are more easily sized, particularly when the complexes must be sized below 0.3 microns, for purposes of filter sterilization. Amino lipids containing unsaturated fatty acids with carbon chain lengths in the range of C₁₄ to C₂₂ may be used. Other scaffolds can also be used to separate the amino group and the fatty acid or fatty alkyl portion of the amino lipid.

In various implementations, amino or cationic lipids used in lipid formulations of the present disclosure may have at least one protonatable or deprotonatable group, such that the lipid is positively charged at a pH at or below physiological pH (e.g., pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the invention. In certain embodiments, the protonatable lipids have a pKa of the protonatable group in the range of 4 to 11, e.g., a pKa of 5 to 7.

The cationic lipid can comprise from 20 mol % to 70 or 75 mol % or from 45 to 65 mol % or 20-25, 25-30, 30-35, 35-40, 40-45, 45-50, 50-55, 55-60, 60-65, or 65-70 mol % of the total lipid present in the particle. In another embodiment, the lipid nanoparticles include from 25% to 75% on a molar basis of cationic lipid, e.g., from 20 to 70%, from 35 to 65%, from 45 to 65%, on a molar basis (based upon 100% total moles of lipid in the lipid nanoparticle). In certain embodiments, the ratio of cationic lipid to nucleic acid is from 3 to 15, such as from 5 to 13 or from 7 to 11.

The lipid-based formulations may als contain a non-cationic lipid. The non-cationic lipid can be a neutral lipid, an anionic lipid, or an amphipathic lipid. Neutral lipids, when present, can be any of a number of lipid species which exist either in an uncharged or neutral zwitterionic form at physiological pH. Such neutral lipids include, for example, diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., lipid particle size and stability of the lipid particle in the bloodstream. Preferably, the neutral lipid is a lipid having two acyl groups (e.g., diacylphosphatidylcholine and diacylphosphatidylethanolamine). In one embodiment, the neutral lipids contain saturated fatty acids with carbon chain lengths in the range of C10 to C20. In another embodiment, neutral lipids with mono or diunsaturated fatty acids with carbon chain lengths in the range of C10 to C20 are used. Additionally, neutral lipids having mixtures of saturated and unsaturated fatty acid chains can be used.

Suitable neutral lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dimyristoyl phosphatidylcholine (D PC), distearoyl-phosphatidyl-ethanolamine (DSPE), SM, 16-O-monomethyl PE, 16-O-dimethyl PE, 18-1-trans PE, 1-stearoyl-2-oleoyl-phosphatidyethanolamine (SOPE), cholesterol, or a mixture thereof. Anionic lipids suitable for use in lipid particles of the invention include, but are not limited to, phosphatidyl lycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

The non-cationic lipid can be from 5 mol % to 90 mol %, 5 mol % to 10 mol %, 5-10, 10-15, 15-20, 20-25, 25-30, 30-35, 35-40, 40-45, 45-50, 50-55, 55-60, 60-65, 65-70, 70-75, 75-80, 80-85, or 85-90 mol % of the total lipid present in the particle. In one embodiment, the lipid nanoparticles include from less than (or from 0.1% to) 15% or less than 45% on a molar basis of neutral lipid, e.g., from 3 to 12% or from 5 to 10% (based upon 100% total moles of lipid in the lipid nanoparticle).

The lipid-based formulations may also contain a sterol such as cholesterol. The sterol can be from 10 mol % to 60 mol % or from 25 mol % to 40 mol % of the lipid particle. In one embodiment, the sterol is 10-15, 15-20, 20-25, 25-30, 30-35, 35-40, 40-45, 45-50, 50-55, or 55-60 mol % of the total lipid present in the lipid particle. In another embodiment, the lipid nanoparticles include from 5% to 50% on a molar basis of the sterol (based upon 100% total moles of lipid in the lipid nanoparticle).

When formulated as a nanoparticle, the lipid nanoparticles may have the structure of a liposome. A liposome is typically a structure having lipid-containing membranes enclosing an aqueous interior. Liposomes preferably have one or more lipid membranes. In certain embodiments, liposomes can be single-layered, referred to as unilamellar, or multi-layered, referred to as multilamellar. When complexed with nucleic acids (e.g., RNA, DNA, the ODN moiety of the compounds of the present disclosure), lipid particles may also be lipoplexes, which are preferably composed of cationic lipid bilayers sandwiched between nucleic acid layers. Liposomes can further be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. In certain embodiments, liposome design may include, but is not limited to, opsonins or ligands in order to improve the attachment of liposomes to unhealthy tissue or to activate events such as, but not limited to, endocytosis. Liposomes may contain a low (e.g. an acidic) or a high (e.g. a basic) pH in order to improve the delivery of the pharmaceutical formulations.

As a non-limiting example, liposomes such as synthetic membrane vesicles may be prepared by the methods, apparatus and devices described in US Patent Publication No. US20130177638, US20130177637, US20130177636, US20130177635, US20130177634, US20130177633, US20130183375, US20130183373 and US20130183372, each of which are herein incorporated by reference in their entirety. In certain implementations, ODN moiety or entire compound may be encapsulated by the liposome, and/or it may be contained in an aqueous core, which may then be encapsulated by the liposome as described in International Pub. Nos. WO2012031046, WO2012031043, WO2012030901 and WO2012006378 and US Patent Publication No. US20130189351, US20130195969 and US20130202684, each of which are herein incorporated by reference in their entirety).

Examples of cancers that can be treated or prevented by the present disclosure include but are not limited to: squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma, myeloproliferative diseases, large B cell lymphoma, and B cell Lymphoma. In certain embodiments, the proliferative disease is melanoma, leukemia, lymphoma, or retinal blastoma.

The methods of the disclosure may comprise administering to the subject a therapeutically effective amount of at least one compound of the disclosure (e.g., Compounds having the structure of formula (I)-(V), Compounds 1-36), which is optionally formulated in a pharmaceutical composition. In certain embodiments, the method further comprises administering to the subject an additional therapeutic agent that treats or prevents cancer. The compound may be a compound for the treatment of a proliferative disease. In certain embodiments, the compound may be a compound for the manufacture of a medicament for the treatment of a proliferative disease. The method for the treatment of a proliferative disease may comprise the administration of a compound or pharmaceutical composition as disclosed herein.

In certain embodiments, administering the compound of the disclosure (e.g., Compounds having the structure of formula (I)-(V), Compounds 1-36) to the subject allows for administering a lower dose of the additional therapeutic agent as compared to the dose of the additional therapeutic agent alone that is required to achieve similar results in treating or preventing a cancer in the subject. For example, in certain embodiments, the compounds disclosed herein may enhance the anti-cancer activity of the additional therapeutic compound, thereby allowing for a lower dose of the additional therapeutic compound to provide the same effect. In certain embodiments, the compounds and the therapeutic agent are co-administered to the subject. In other embodiments, the compound of the disclosure and the therapeutic agent are coformulated and co-administered to the subject.

In certain embodiments, the subject is a mammal. In other embodiments, the mammal is a human.

The therapeutically effective amount or dose of a compound of the present disclosure depends on the age, sex and weight of the patient, the current medical condition of the patient and the progression of a cancer in the patient being treated. The skilled artisan is able to determine appropriate dosages depending on these and other factors. For example, the therapeutically effective amount may be determined based on the amount of PROTAC required to treat a proliferative disease of interest. For example, the therapeutically effective amount may be based on more than 20% or more than 30% or more than 40% or more than 50% or more than 60% or more than 70% or more than 80% or more than 90% or more than 95% or more than 95% or more than 99% or 100% being decaged following endocytosis f the compounds described herein by weight of the composition.

For example, a suitable dose of a compound of the present disclosure may be in the range of from 0.01 mg to 5,000 mg per day, such as from 0.1 mg to 1,000 mg, for example, from 1 mg to 500 mg, such as 5 mg to 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with, for example, a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the disclosure is optionally given continuously; alternatively, the dose of drug being administered is temporarily reduced or temporarily suspended for a certain length of time. The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds for use in the method of the disclosure (e.g., Compounds having the structure of formula (I)-(V), Compounds 1-36) may be formulated in unit dosage form. Typically, unit dosage forms are physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., 1 to 4 or more times per day). When multiple daily doses are used, the unit dosage form may be the same or different for each dose.

Many applications of the compositions of the present disclosure may involve cell surface localization or targeting. However, the general methods of this invention may also be extended to include internalization of the compound in cells. In these embodiments, constructs that incorporate an internalization or targeting moiety that causes the cell to internalize a PROTAC that only becomes active following the internatlization. In this way, targeted internalization occurs instead of the non-specific internalization that would occur by use of the internalization agent and/or a localization agent.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application. Unless otherwise apparent from context, the sum of all weight percentages should not exceed 100% and all percentages in relation to components refer to percentages by weight of the composition.

### EXAMPLES

The following examples illustrate specific aspects of the instant description. The examples should not be construed as limiting, as the example merely provides specific understanding and practice of the embodiments and its various aspects.

### Example 1: NF-κB Transcription Factor Degradation

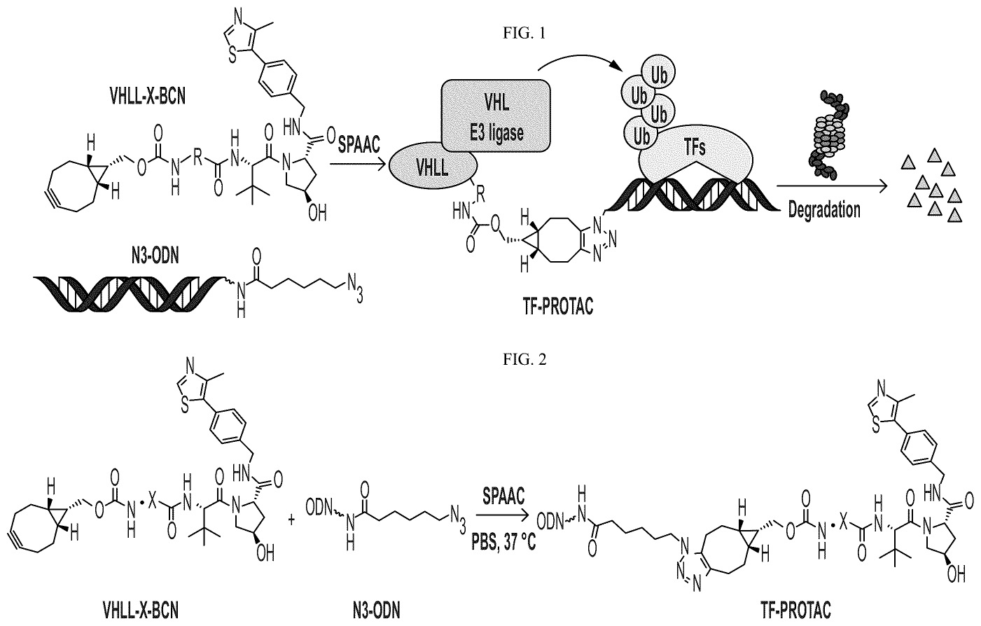

The present disclosure involves the development of a generalizable platform of DNA-PROTAC by using commercially available azide-modified DNA oligomer (N3-ODN) to chemically click on the BCN-modified VHL ligand with a series of linkers with different lengths and polarities (VHLL-X-BCN, FIG. 1). After a simple purification process to remove excess ligand, the TF-PROTAC is ready for transfection into cells, which enables targeted degradation of specific TFs based their respective DNA-binding motif (FIGS. 1 and 2).

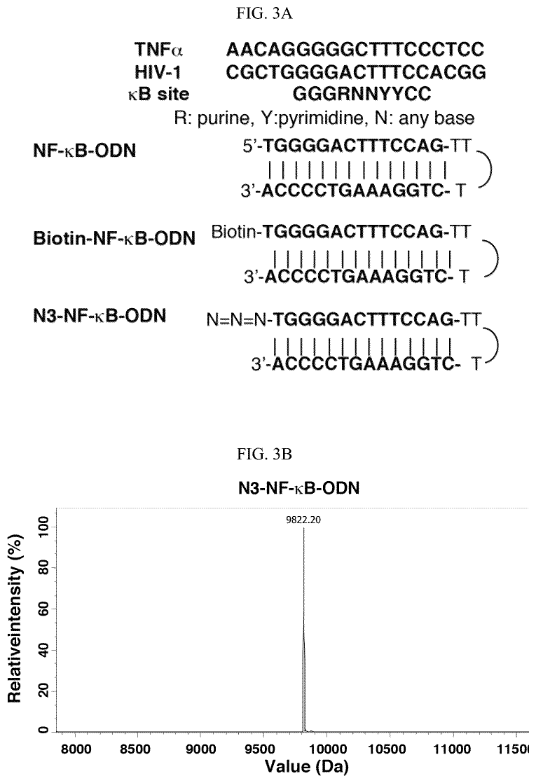

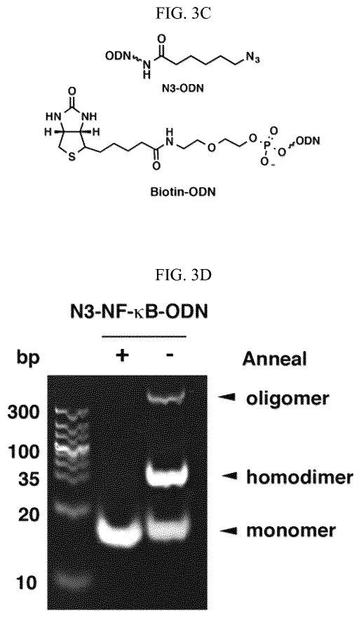

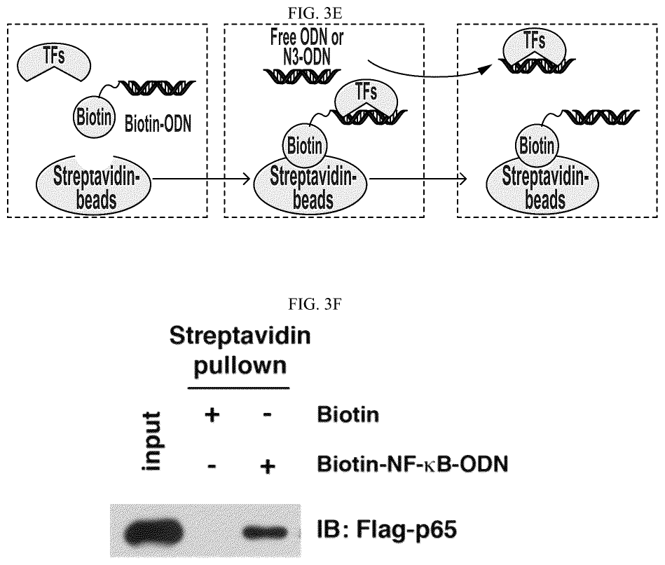

To experimentally evaluate this unique PROTAC platform, the canonical NF-κB (nuclear factor kappa-light-chain-enhancer of activated B cells) was chosen as a target because the binding motif of NF-κB is well-defined and has been extensively validated in vitro and in vivo, as detailed in, for example, Nabel, G .; et al Nature 326.6114 (1987): 711-3 and Bielinska, A. et al Science 250.4983 (1990): 997-1000, each of which are hereby incorporated by reference in their entirety. The NF-κB motif GGGRNNYYCC (SEQ ID NO: 1) (R is purine, Y is pyrimidine, N is any base) is shared by many of its transcriptional targets, such as TNFα and HIV-1 (FIG. 3A). Thus, we synthesized a single strand DNA oligonucleotide, 5′-TGGGGACTTTCCAGTTTCTGGAAAGTCCCCA-3′ (SEQ ID NO: 4) (NF-κB decoy, hereafter termed as NF-κB-ODN), which forms a double-strand hairpin structure via intra-dimerization and therefore confers resistance to exonucleases in cells (FIGS. 3B and 3C).

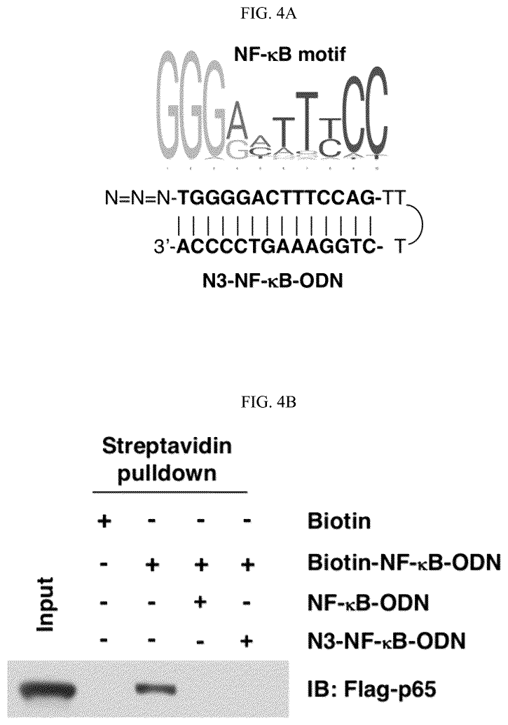

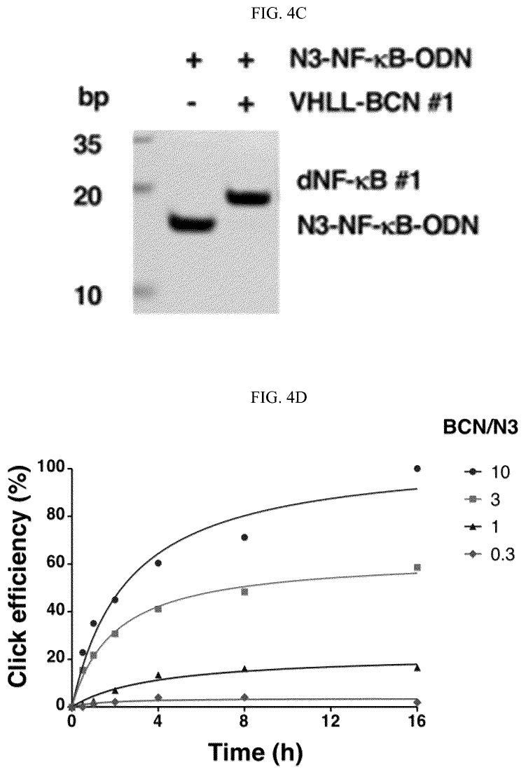

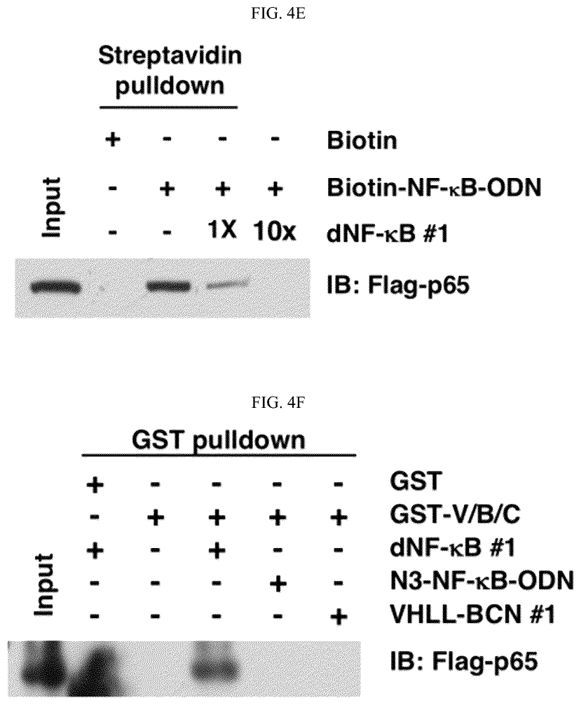

For the purpose of conjugation of an E3 ligand on the end of NF-κB-ODN, an azide modification version of NF-κB-ODN (hereafter termed as N3-NF-κB-ODN) was further synthesized by incorporating an azide group on the 5′ end of NF-κB-ODN through the 5′ amino modifier C₆ (FIGS. 4A, 3A-3C). The N3-NF-κB-ODN forms homodimer and oligomer via inter-dimerization, while only the annealing step ensures the entire intra-dimerization process (FIG. 3D).

To determine whether NF-κB-ODN binds with NF-κB properly, we also synthesized a biotin-modified version of NF-κB-ODN (hereafter termed as Biotin-NF-κB-ODN, FIGS. 3A-3C). Biotin-NF-κB-ODN was capable of binding with p65, the DNA binding subunit of NF-κB heterodimer (FIGS. 3E and 3F), in a streptavidin pulldown assay. On the other hand, excess NF-κB-ODN and N3-NF-κB-ODN competed with Biotin-NF-κB-ODN for the binding with p65 (FIG. 4B and 3F), indicating that the azide modification will likely not affect the binding ability of NF-κB-ODN with NF-κB in this experimental setting.

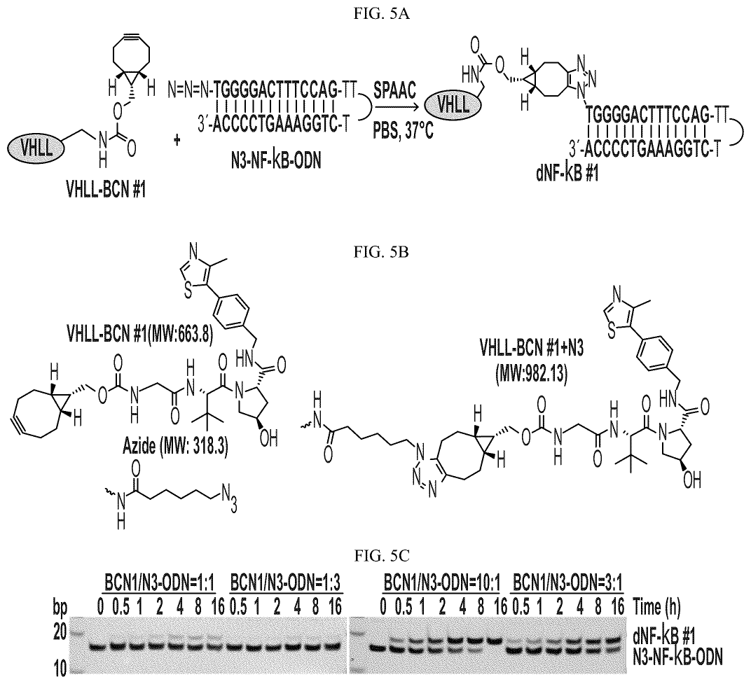

In order to synthesize the TF-PROTACs bearing DNA oligo binding motifs, the robust biorthogonal reaction, copper-free strain-promoted azide-alkyne cycloaddition (SPAAC) reaction was employed. First, a bicyclooctyne (BCN) group was conjugated with a VHL ligand (VH-032) via different linkers (hereafter termed as VHLL-X-BCN, FIG. 1 and Supporting Scheme S1). Followed by a SPAAC reaction between the resulting BCN modified VHL ligands and N3-TF-ODN under physiological conditions afforded TF-PROTACs (FIG. 5A).

To assess the efficiency of the SPAAC reaction for N3-ODN, the amount of click product of the reaction between VHLL-BCN #1 (X is CH₂) and N3-NF-κB-ODN under physiological conditions (PBS, 37° C.) was monitored. Notably, after the incorporation of VHLL-BCN to N3-NF-κB-ODN, the molecule weight will increase around 660 Da, which could be clearly separated by 20% native polyacrylamide gel electrophoresis (PAGE, FIGS. 4C, 5B and 5C).

Increasing the amount of VHLL-BCN #1 and the reaction time improved the efficiency of the SPAAC reaction. Furthermore, the click efficiency could be obtained up to more than 60% with ten folds excess of VHLL-BCN #1 after 4 hours reaction (FIGS. 4D, 5B, and 5C). Moreover, the click product VHLL-NF-κB-ODN (hereafter termed as dNF-κB) competed with Biotin-NF-κB for the binding to p65 (FIG. 4E), indicating that dNF-κB retains the affinity with NF-κB and thus is suitable for the PROTAC design.

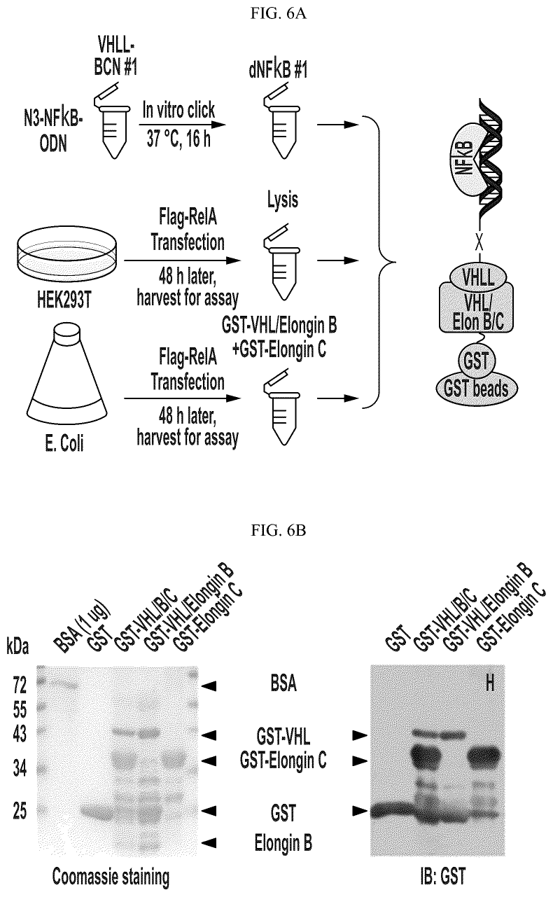

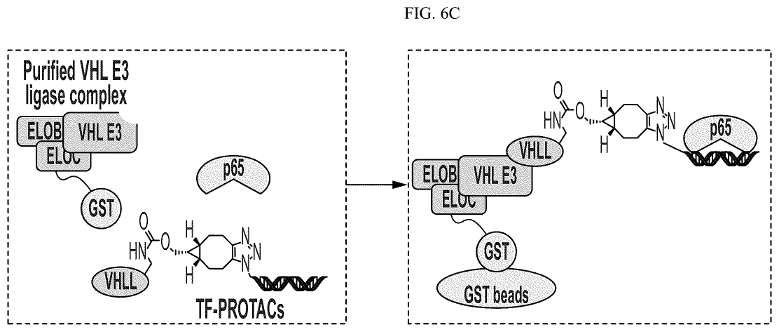

Furthermore, dNF-κB is capable of recruiting VHL E3 ubiquitin ligase to POI, NF-κB. Recombinant GST-VHL protein was purified from E. Coli on GST-agarose beads, which was further incubated with cell lysis derived from HEK293T cells that ectopically expressed p65 protein in the presence of dNF-κB (FIGS. 6A-6C). dNF-κB mediated the interaction between p65 and VHL E3 ubiquitin ligase (FIG. 2E), thus indicating that TF-PROTAC might be used for degradation of DNA binding proteins, such as TFs. However, dNF-κB obtained in current stage could not efficiently promote the degradation of p65 in cells, which prompted further optimization of the length of linker between the VHL ligand and DNA oligomer moieties for achieving efficient destruction of NF-κB in cells.

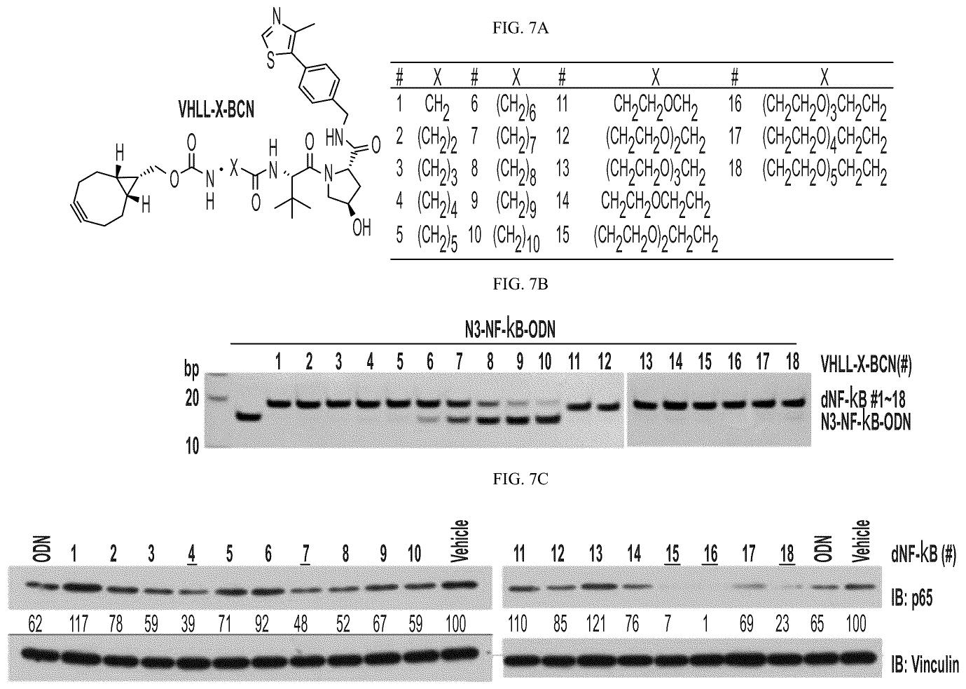

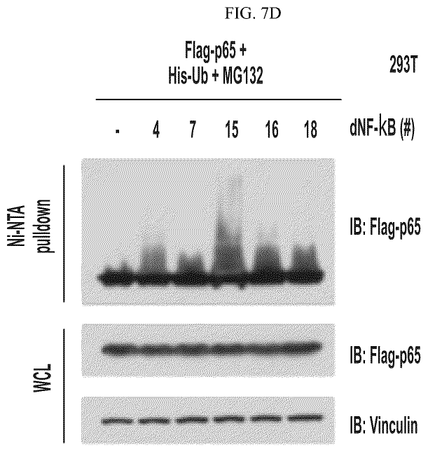

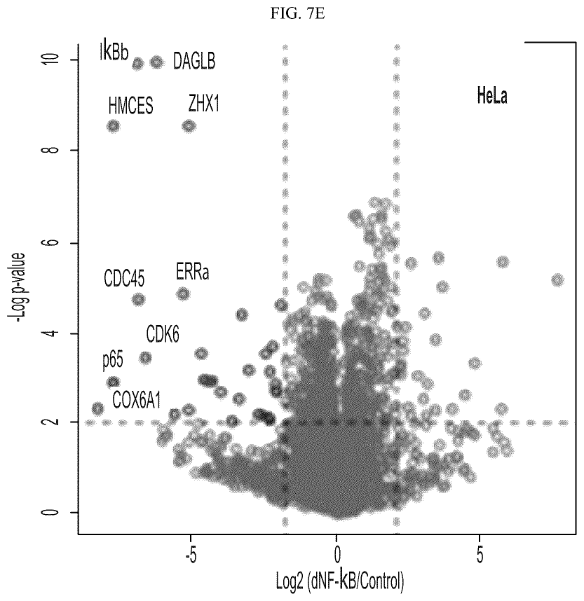

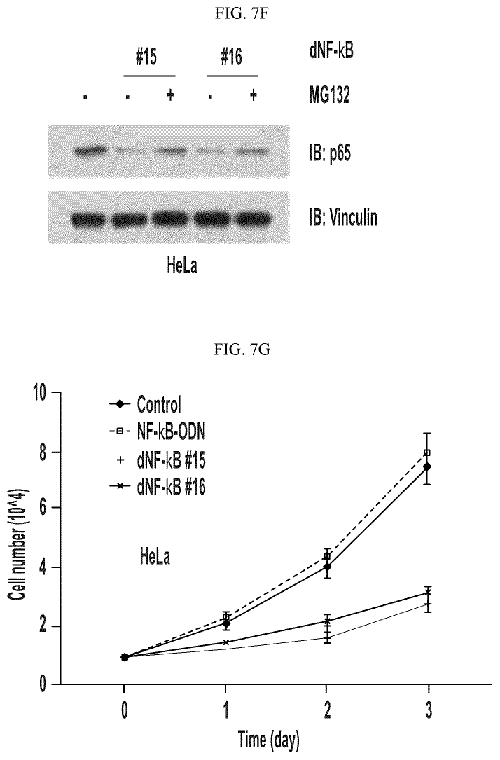

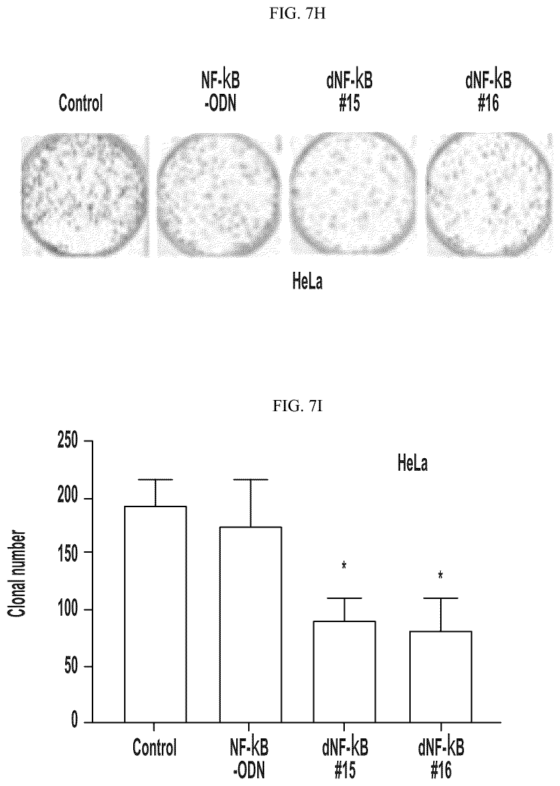

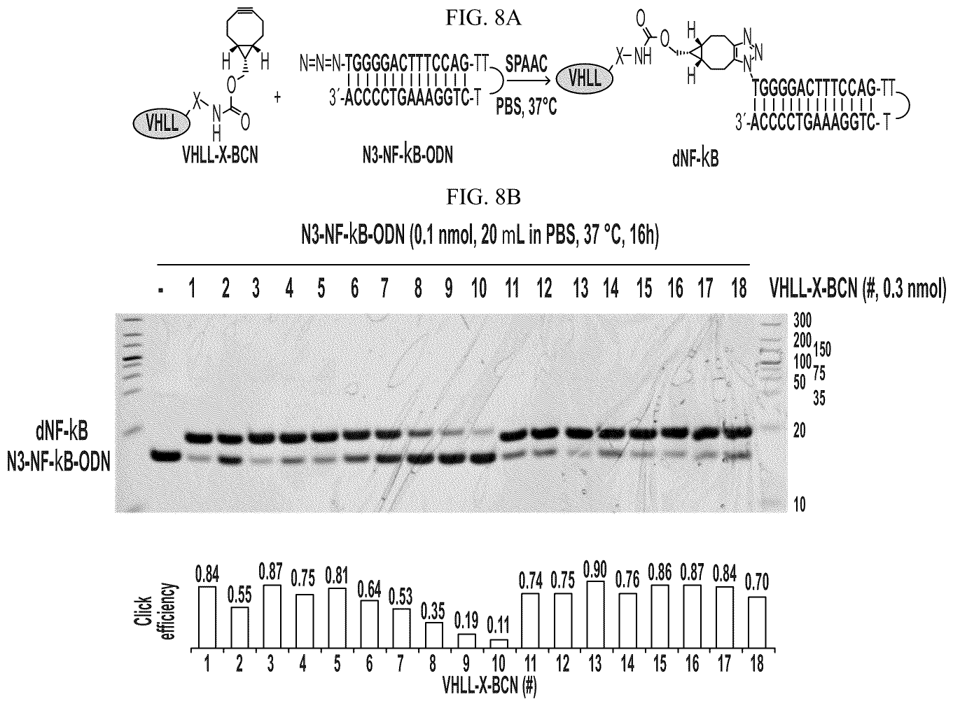

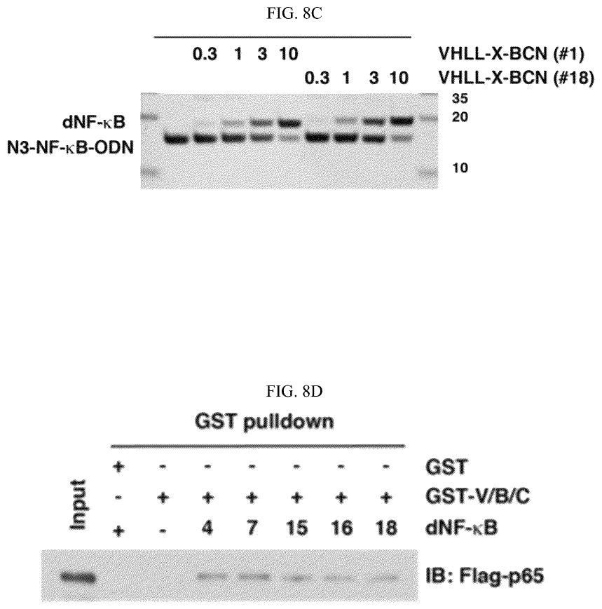

Another 17 BCN-modified VHL ligands, with different length and polarity (FIG. 7A). Similar as VHLL-BCN #1, these 17 modified VHL ligand could be chemically clicked onto azide-modified DNA oligomers under physiological conditions (FIG. 8A). The click efficiency of VHLL-BCN #2-5 and #11-18 was as high as VHLL-BCN #1 (FIGS. 7B, 8B and 8C). However, the click efficiency appears to gradually reduce when the length of the alkyl chain increased as well (FIGS. 7B, 8B and 8C). Furthermore, when transfected into Hela cells with liposome, five of the eighteen NF-κB degraders led to a reduction of endogenous p65 protein abundance to less than 50% in comparison to control cells (FIG. 7C), with dNF-κB #15 and #16 displaying the best degradation efficiency.

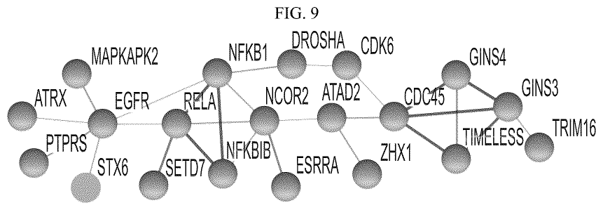

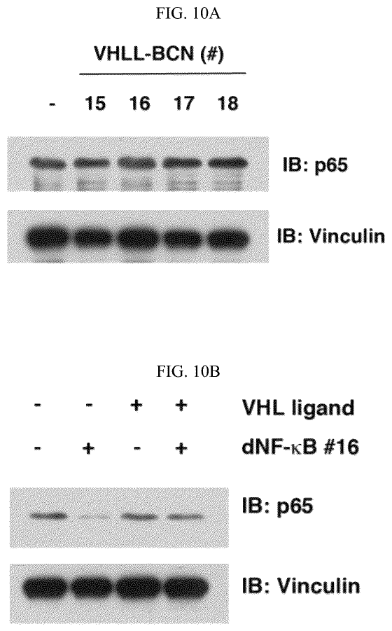

Moreover, in vivo pulldown assay and ubiquitination assay indicated that in keeping with their degradation capacity, these dNF-κB degraders led to binding between VHL E3 ligase and p65 (FIG. 8D) and efficient ubiquitination of p65 in cells (FIG. 7D). In an unbiased global proteomic study using quantitative mass spectrometry, dNF-κB #16 led to significant degradation of p65 (FIG. 7F). Moreover, the majority of the 31 proteins with a reduced expression level in dNF-κB-treated samples, out of more than 4,100 detected proteins, had a physical or functional relationship with p65 (FIGS. 7E and 9), including the p65-binding partners IKBB and CDK6. This suggests that PROTACs comprising an ODN capable of binding to p65 are relative selective for p65.

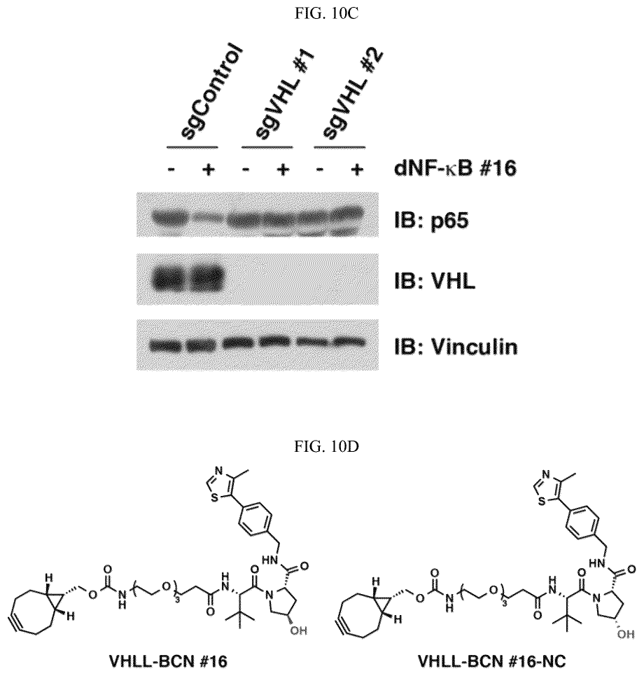

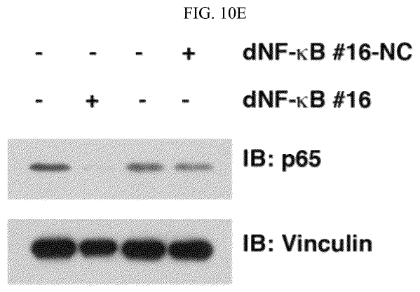

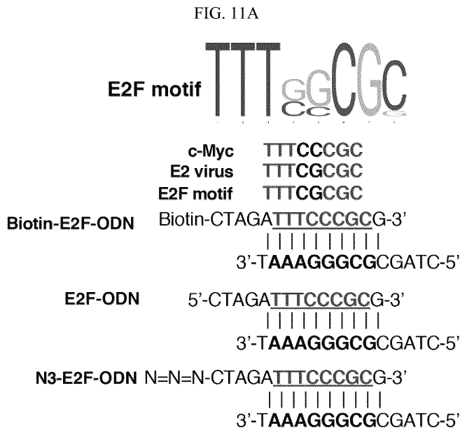

To determine whether the degradation of p65 is due to an on-target effect of the TF-PROTAC rather than a non-specific effect from the VHLL-BCN, HeLa cells were treated with several VHLL-BCN compounds. These compounds were incapable of reducing p65 (FIG. 10A). Furthermore, the degradation of p65 by NF-κB degraders could be blocked by co-treatment with the proteasome inhibitor MG132 (FIG. 3F) or the VHL ligand VH-032 (FIG. 10B). It was also determined that dNF-κB could not degrade p65 in Hela cells after depletion of the endogenous VHL. E3 ubiquitin ligase (FIG. 10C).

In addition, because the hydroxyl group in the VHL ligand moiety of dNF-κB is critical for binding the VHL E3 ubiquitin ligase, a negative control (referred to herein as VHLL-BCN #16-NC) was synethsized by inversion of the stereochemistry of the hydroxyl group from R to S (FIG. 10D). dNF-κB #16-NC, which resulted from the SPAAC reaction of VHLL-BCN#16-NC and N3-NF-κB-ODN, could not degrade p65 (FIG. 10E).

These results indicate that TF-PROTACs likely lead to the degradation of TFs such as p65 in a VHL- and proteasome-dependent manner. As a result, treatment with dNF-κB (#15 and 16), but not NF-κB-ODN, inhibited the proliferation (FIG. 7G) and tumorigenesis ability (FIGS. 7H-7I), presumably via promotion of the degradation of oncogenic TF NF-κB.

### Example 2: E2F Transcription Factor Degradation

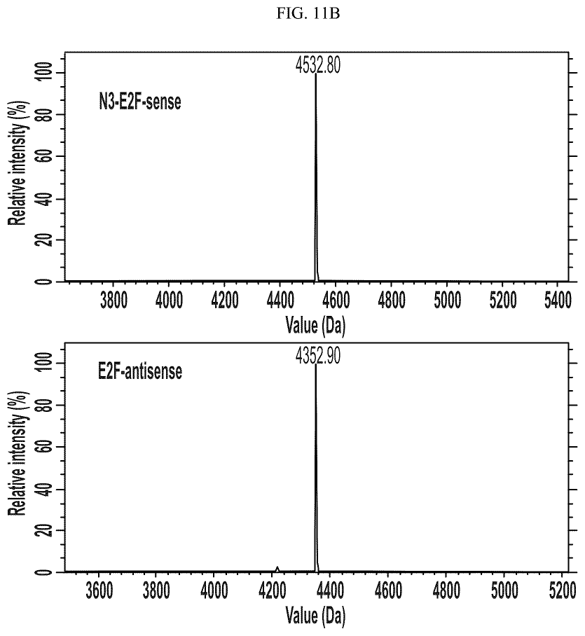

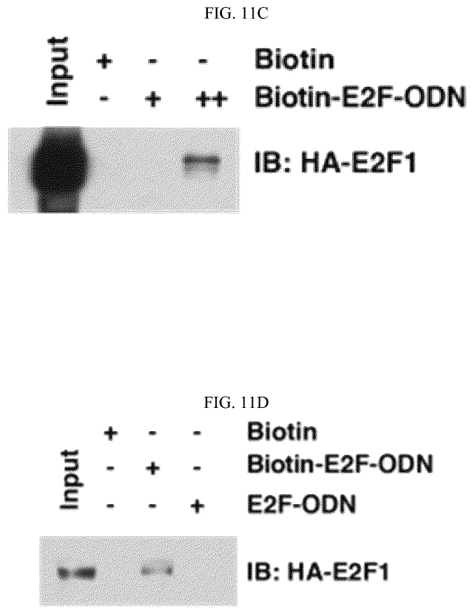

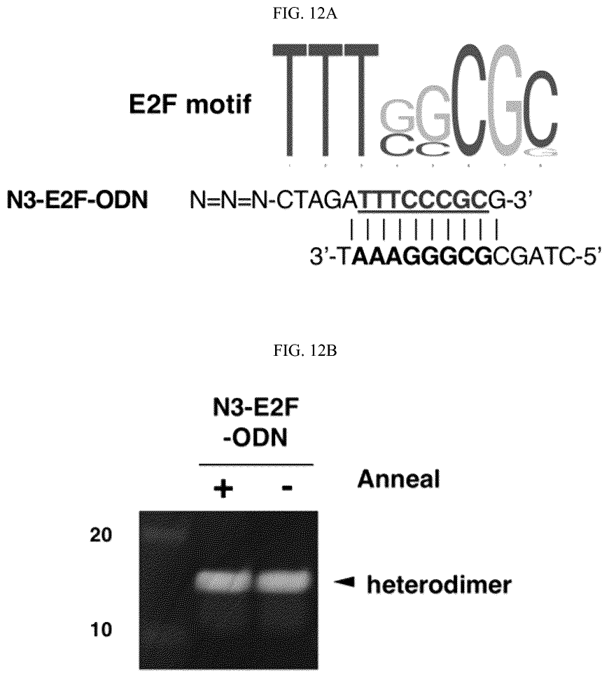

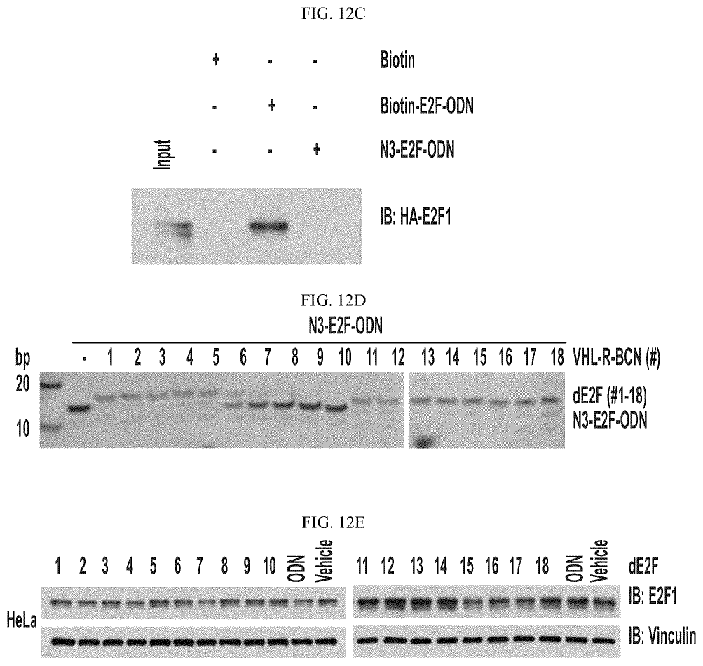

To determine whether the design of TF-PROTACs could be applied to other transcription factors besides NF-κB, DNA 15-mers contain a E2F binding motif (TTTG/CG/CCGC) from MYC promoter were designed and synthesized. In these PROTACs, the ODN group had a sense chain as 5′-CTAGATTTCCCGCG-3′ (SEQ ID NO: 5) and the anti-sense chain as 5′-CTAGCGCGGAAAT-3′ (SEQ ID NO: 7), hereafter named as E2F-ODN (FIG. 11A). Similar to the NF-κB experiments, azide-modified E2F-ODN (hereafter termed as N3-E2F-ODN) and biotin-modified E2F-ODN (hereafter termed as Biotin-E2F-ODN, FIGS. 10A, 11B, 12A, and 13A) were synthesized. After annealing in vitro, the sense and antisense oligomers formed double-stained heterodimer, which could be quality controlled by separation in native PAGE (FIG. 12B). Using the competitive binding assay, we found that N3-E2F-ODN bound with the E2F1 transcription factor as efficiently as E2F-ODN did in this experimental setting (FIGS. 12C and 11C-11D).

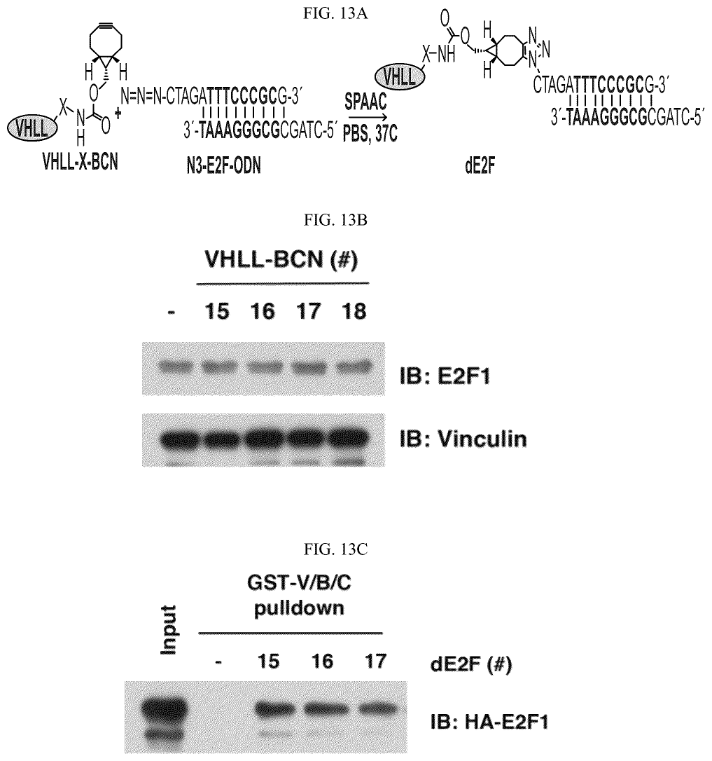

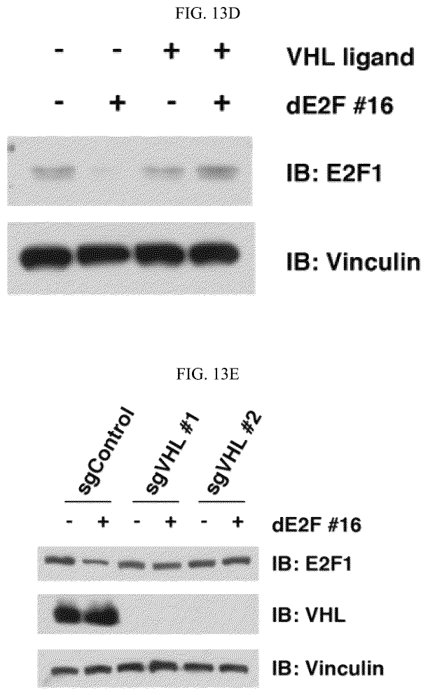

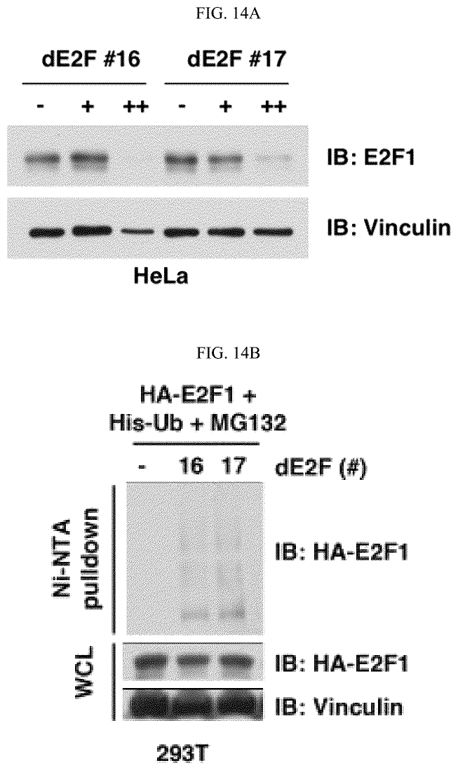

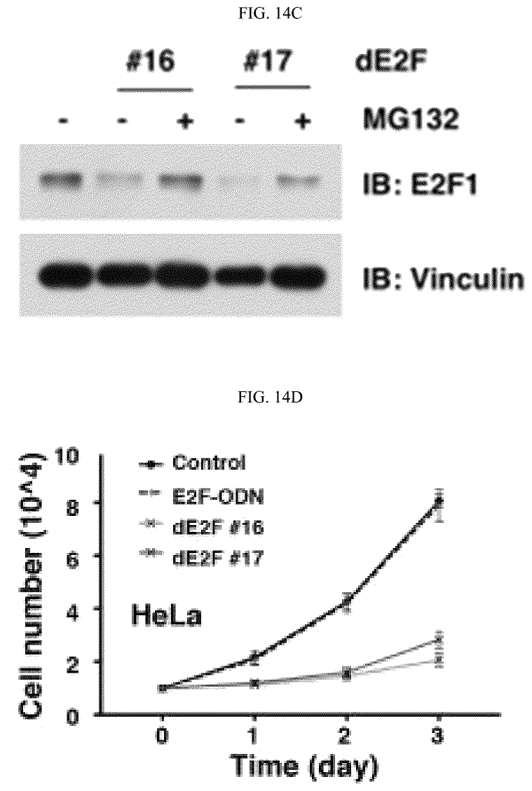

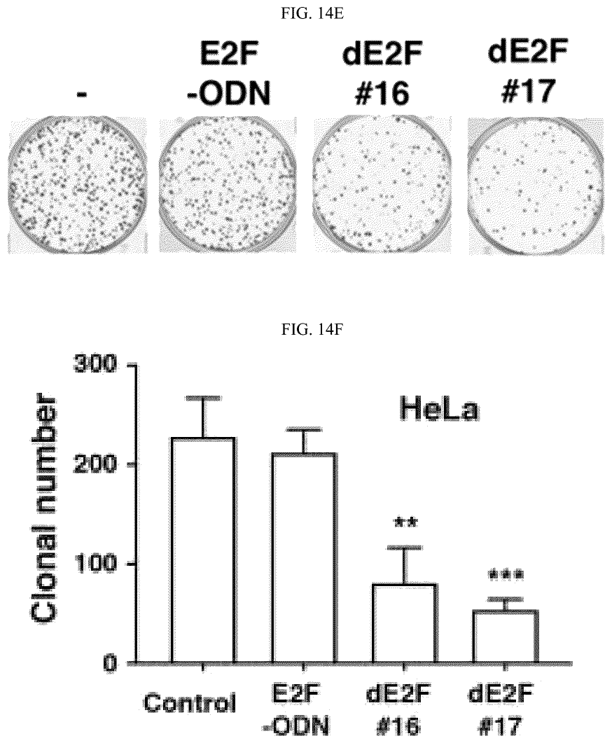

Next, the click efficiency between the 18 BCN-modified VHL ligands (VHLL-BCN #1-18) and N3-E2F-ODN (FIGS. 12D and 13A) was measured. Notably, VHLL-BCN #1-5 and # 11-18 were efficiently clicked with N3-E2F-ODN, while #6-10 did not (FIG. 12D). By transfection of E2F1 degraders into HeLa cells with a liposomal delivery system, in vivo degradation results indicated that 2 of the eighteen E2F1 degraders (#16 and #17) led to a noticeable reduction of endogenous E2F1 protein abundance (FIGS. 12E and 14A), while the VHLL-BCN compound (#15-#18) could not (FIG. 13B). Additionally, dE2F1 (#16 and #17) induced the binding between the VHL E3 ubiquitin ligase and E2F1 (FIG. S9C), which led to ubiquitinization in cells (FIG. 14B). Moreover, the degradation of E2F1 by dE2F1 #16 and #17 could be blocked by the proteasome inhibitor MG132 (FIG. 14B), and both dE2F1 #16 and #17 led to binding between VHL E3 ligase and E2F1 (FIG. 11B), the VHL ligand VH-032 (FIG. 13D) or depletion of the endogenous VHL E3 ligase. Accordingly, these E2F1 TF-PROTACs function in a VHL- and proteasome-dependent manner. In keeping with this notion, treatment with dE2F #16 and #17 inhibited the proliferation (FIG. 14D) and tumorigenesis ability (FIGS. 14E-14F).

Recently, Shao, J. et al., “Destruction of DNA-binding proteins by programmable O'PROTAC: Oligonucleotide-based PROTAC”. bioRxiv 2021, 2021.03.08.434493, which is hereby incorporated by reference in its entirety, reported a similar DNA-PROTACs design, namely O′PROTAC, which is capable of degrading other TFs, such as ERG and LEF1. Compared with O′PROTAC, the design of the PROTACs in present disclosure (in particular comparison of synthetic routes and surprising linker effects detailed herein) has the advantage of easy assembly and generalization by: 1) a serial of BCN-modified VHL ligands with 18 different linker length and polarity to optimize the linker moiety for efficient degradation of TFs in cells; 2) commercial availability of azide-modified DNA oligomers (either single strain or double strain) to be expanded to any TF of interest. Taken together, this disclosure provides a generalizable screening strategy for TF-PROTACs to targeted degrade any given transcription factors with known DNA binding motif. These results validate two lead TF-PROTACs (dNF-κB and dE2F) that effectively degraded p65 and E2F1, respectively, in a proteasome-dependent manner in cells. Furthermore, these results demonstrate that this unique approach is generalizable and could be applied to all other TFs with known motif, thereby largely extent the spectrum of PROTACs to additional up to 1300 drug targets, with immediate clinic benefits to various human diseases.

### Experimental Methods: Compound Syntheses

Common materials or reagents were purchased from commercial sources and used without further purification. High performance liquid chromatography (HPLC) spectra were acquired using an Agilent 1200 Series system with DAD detector for all the intermediates and final products below. Chromatography was performed on a 2.1×150 mm Zorbax 300SB-C_{18 5} um column with water containing 0.1% formic acid as solvent A and acetonitrile containing 0.1% formic acid as solvent B at a flow rate of 0.4 mL/min. The gradient program was as follows: 1% B (0-1 min), 1-99% B (1-4 min), and 99% B (4-8 min). High-resolution mass spectra (HRMS) data were acquired in positive ion mode using an Agilent G1969A API-TOF with an electrospray ionization (ESI) source. Ultra-performance liquid chromatography (UPLC) spectra for compounds were acquired using a Waters Acquity I-Class UPLC system with a PDA detector. Chromatography was performed on a 2.1 Å˜30 mm ACQUITY UPLC BEH C_{18 1.7} um column with water containing 3% acetonitrile, 0.1% formic acid as solvent A and acetonitrile containing 0.1% formic acid as solvent B at a flow rate of 0.8 mL/min. The gradient program was as follows: 1-99% B (1-1.5 min), and 99-1% B (1.5-2.5 min). Nuclear Magnetic Resonance (NMR) spectra were acquired on a Bruker DRX-600 spectrometer with 600 MHz for proton (1H NMR) and 151 MHz for carbon (13C NMR); chemical shifts are reported in (8). Preparative HPLC was performed on Agilent Prep 1200 series with UV detector set to 220 or 254 nm. Samples were injected onto a Phenomenex Luna 250×30 mm, 5 μm, C₁₈ column at room temperature. The flow rate was 40 mL/min. A linear gradient was used with 10% of acetonitrile in H2O (with 0.08% NH₄HCO₃) (B) to 100% of acetonitrile (A). HPLC was used to test the purity of target compounds. All of the final compounds had >96% purity using the HPLC methods described above. The 18 VHL ligand liners, VHLL 1-18, were synthesized according to the published procedures such as those described in WO 2020173440, which is hereby incorporated by reference in its entirety. VH-032 was synthesized according to the published procedures in Galdeano C, et al J. Med. Chem. 57.20 (2014): 8657-8663, which is hereby incorporated by reference in its entirety and particularly in relation to synthesis of VH-032.

Supporting Scheme S1, general method for synthesis of VHLL-BCN #1˜#18

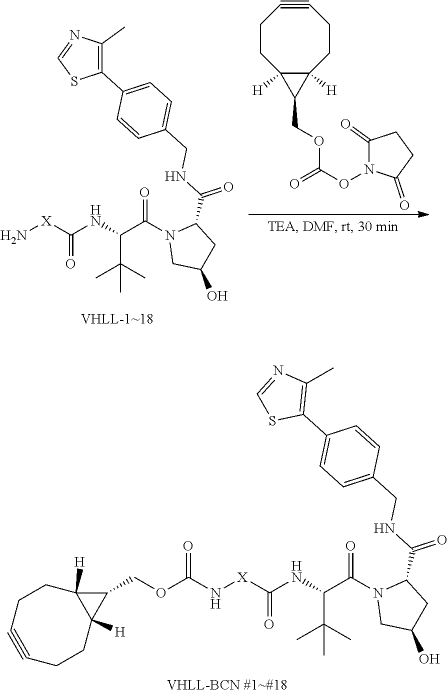

((1R,8S,9s)-bicyclo[6.1.0]non-4-yn-9-yl)methyl (2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethyl)carbamate (VHLL-BCN #1)

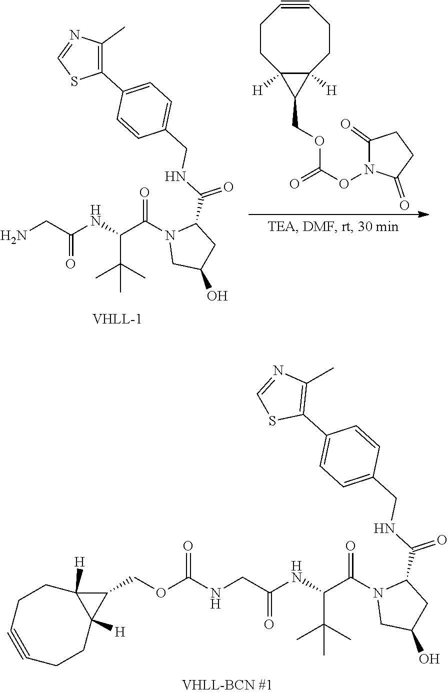 [SMILES: Cc1ncsc1-c1ccc(CNC(=O)[C@@H]2C[C@@H](O)CN2C(=O)[C@@H](NC(=O)CN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CNC(=O)OC[C@@H]2[C@@H]3CCC#CCC[C@@H]32)C(C)(C)C)cc1]

To a solution of (2S,4R)-1-((S)-2-(2-aminoacetamido)-3,3-dimethylbutanoyl)-4-hydroxy-N-(4-(4-methylthiazol-5-yl)benzyl)pyrrolidine-2-carboxamide (VHLL-1) (12.7 mg, 0.018 mmol, 1.2 equiv) and ((1R,8S,9s)-bicyclo[6.1.0]non-4-yn-9-yl)methyl (2,5-dioxopyrrolidin-1-yl) carbonate (4.4 mg, 0.015 mmol, 1.0 equiv) in DMF (1 mL) was added triethylamine (TEA) (4.5 mg, 0.045 mmol, 3.0 equiv) at room temperature. After being stirred at room temperature for 30 min, the resulting mixture was purified by preparative HPLC (10%-100% acetonitrile/0.08% NH₄HCO₃ in H₂O). All of the product fractions were collected and extracted with EA (3×20 mL). The organic layers were combined, washed with water (20 mL) and brine (20 mL), dried over Na₂SO₄, and concentrated to afford compound VHLL-BCN #1 as white solid (7.2 mg, 72%). ¹H NMR (600 MHZ, Methanol-d₄) δ 8.78 (s, 1H), 7.37 (d, J=8.1 Hz, 2H), 7.31 (d, J=8.1 Hz, 2H), 4.55 (s, 1H), 4.49-4.42 (m, 2H), 4.41-4.36 (m, 1H), 4.25 (d, J=15.4 Hz, 1H), 4.13 -4.03 (m, 2H), 3.77 (d, J=11.0 Hz, 1H), 3.72-3.67 (m, 3H), 2.38 (s, 3H), 2.17-1.90 (m, 10H), 1.55-1.46 (m, 2H), 1.33-1.20 (m, 1H), 0.93 (s, 9H), 0.87-0.78 (m, 2H). ESI m/z=664.4 [M+H]⁺.

((1R,8S,9s)-bicyclo[6.1.0]non-4-yn-9-yl)methyl (3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropyl)carbamate (VHLL-BCN #2)

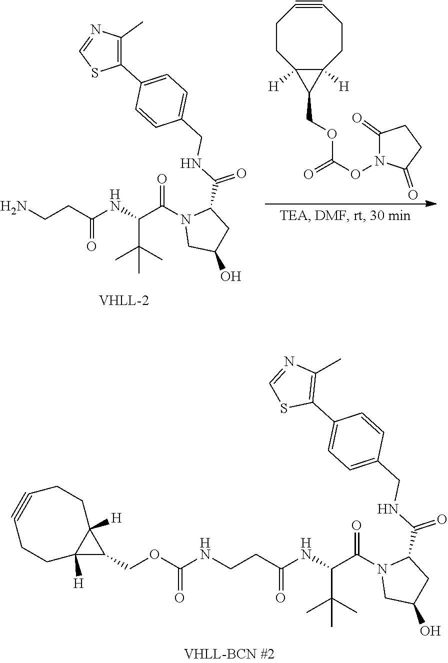 [SMILES: Cc1ncsc1-c1ccc(CNC(=O)[C@@H]2C[C@@H](O)CN2C(=O)[C@@H](NC(=O)CCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCNC(=O)OC[C@@H]2[C@@H]3CCC#CCC[C@@H]32)C(C)(C)C)cc1]

VHLL-BCN #2 was synthesized following the standard procedure for preparing VHLL-BCN #1 from (2S,4R)-1-((S)-2-(3-aminopropanamido)-3,3-dimethylbutanoyl)-4-hydroxy-N-(4-(4-methylthiazol-5-yl)benzyl)pyrrolidine-2-carboxamide (VHLL-2) and obtained as white solid (8.1 mg, 80%). ¹H NMR (600 MHZ, Methanol-d₄) δ 8.89 (s, 1H), 7.48 (d, J=8.3 Hz, 2H), 7.43 (d, J=8.1 Hz, 2H), 4.63 (s, 1H), 4.61-4.53 (m, 2H), 4.53-4.47 (m, 1H), 4.36 (d, J=15.5 Hz, 1H), 4.14 (d, J=8.3 Hz, 2H), 3.93 (d, J=11.0 Hz, 1H), 3.81 (dd, J=11.0, 3.9 Hz, 1H), 3.43 -3.34 (m, 2H), 2.56-2.40 (m, 5H), 2.30-2.03 (m, 10H), 1.68-1.57 (m, 2H), 1.44-1.27 (m, 1H), 1.05 (s, 9H), 0.97-0.88 (m, 2H). ESI m/z=678.5 [M+H]⁺.

((1R,8S,9s)-bicyclo[6.1.0]non-4-yn-9-yl)methyl (4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-4-oxobutyl)carbamate (VHLL-BCN #3)

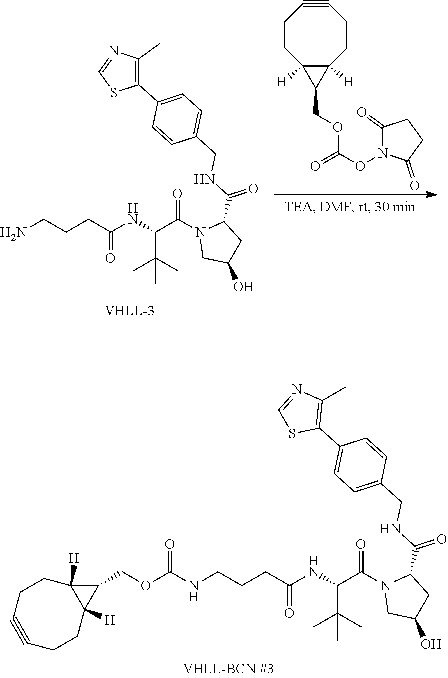 [SMILES: Cc1ncsc1-c1ccc(CNC(=O)[C@@H]2C[C@@H](O)CN2C(=O)[C@@H](NC(=O)CCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCCNC(=O)OC[C@@H]2[C@@H]3CCC#CCC[C@@H]32)C(C)(C)C)cc1]

VHLL-BCN #3 was synthesized following the standard procedure for preparing VHLL-BCN #1 from (2S,4R)-1-((S)-2-(4-aminobutanamido)-3,3-dimethylbutanoyl)-4-hydroxy-N-(4-(4-methylthiazol-5-yl)benzyl)pyrrolidine-2-carboxamide (VHLL-3) and obtained as white solid (7.9 mg, 76%). ¹H NMR (600 MHZ, Methanol-d₄) δ 8.89 (s, 1H), 7.48 (d, J=6.3 Hz, 2H), 7.43 (d, J=8.2 Hz, 2H), 4.70-4.53 (m, 3H), 4.52-4.48 (m, 1H), 4.37 (d, J=15.6 Hz, 1H), 4.15 (d, J=8.3 Hz, 2H), 3.92 (d, J=10.5 Hz, 1H), 3.82 (d, J=10.7 Hz, 1H), 3.20-3.07 (m, 2H), 2.49 (s, 3H), 2.38-2.04 (m, 12H), 1.86-1.73 (m, 2H), 1.68-1.55 (m, 2H), 1.43-1.35 (m, 1H), 1.06 (s, 9H), 0.98-0.85 (m, 2H). ESI m/z=692.4 [M+H]⁺.

((1R,8S,9s)-bicyclo[6.1.0]non-4-yn-9-yl)methyl (5-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-5-oxopentyl)carbamate (VHLL-BCN #4)

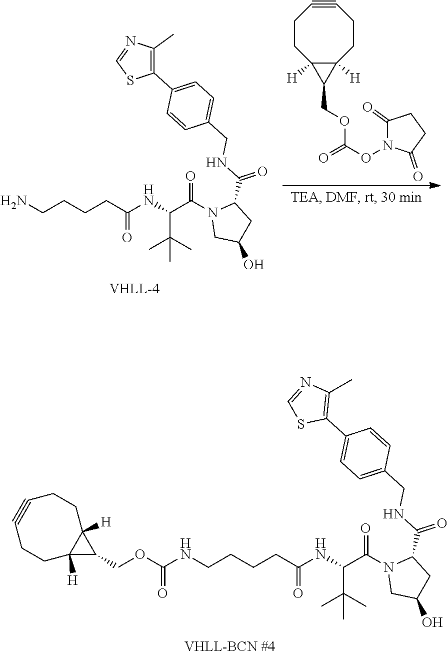 [SMILES: Cc1ncsc1-c1ccc(CNC(=O)[C@@H]2C[C@@H](O)CN2C(=O)[C@@H](NC(=O)CCCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCCCNC(=O)OC[C@@H]2[C@@H]3CCC#CCC[C@@H]32)C(C)(C)C)cc1]

VHLL-BCN #4 was synthesized following the standard procedure for preparing VHLL-BCN #1 from (2S,4R)-1-((S)-2-(5-aminopentanamido)-3,3-dimethylbutanoyl)-4-hydroxy-N-(4-(4-methylthiazol-5-yl)benzyl)pyrrolidine-2-carboxamide (VHLL-4) and obtained as white solid (9.1 mg, 86%). ¹H NMR (600 MHZ, Methanol-d₄) δ 8.89 (s, 1H), 7.50-7.46 (m, 2H), 7.45 -7.41 (m, 2H), 4.64 (s, 1H), 4.61-4.53 (m, 2H), 4.53-4.48 (m, 1H), 4.37 (d, J=15.4 Hz, 1H), 4.14 (d, J=7.8 Hz, 2H), 3.92 (d, J=10.9 Hz, 1H), 3.86-3.79 (m, 1H), 3.14-3.09 (m, 2H), 2.49 (s, 3H), 2.39-2.06 (m, 12H), 1.68-1.56 (m, 4H), 1.55-1.48 (m, 2H), 1.42-1.33 (m, 1H), 1.05 (s, 9H), 0.98-0.86 (m, 2H). ESI m/z=706.4 [M+H]⁺.

((1R,8S,9s)-bicyclo[6.1.0]non-4-yn-9-yl)methyl (6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-6-oxohexyl)carbamate (VHLL-BCN #5)

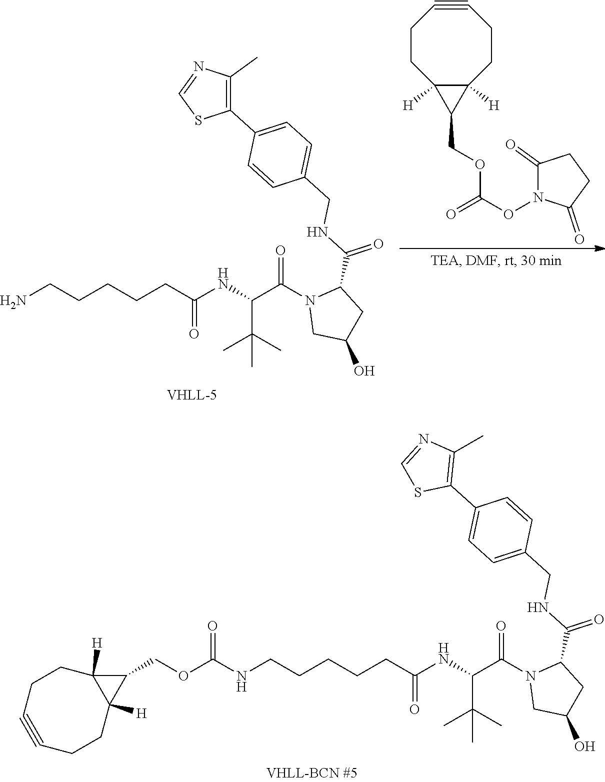 [SMILES: Cc1ncsc1-c1ccc(CNC(=O)[C@@H]2C[C@@H](O)CN2C(=O)[C@@H](NC(=O)CCCCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCCCCNC(=O)OC[C@@H]2[C@@H]3CCC#CCC[C@@H]32)C(C)(C)C)cc1]

VHLL-BCN #5 was synthesized following the standard procedure for preparing VHLL-BCN #1 from (2S,4R)-1-((S)-2-(6-aminohexanamido)-3,3-dimethylbutanoyl)-4-hydroxy-N-(4-(4-methylthiazol-5-yl)benzyl)pyrrolidine-2-carboxamide (VHLL-5) and obtained as white solid (8.4 mg, 78%). ¹H NMR (600 MHZ, Methanol-d₄) δ 8.89 (s, 1H), 7.48 (d, J=8.0 Hz, 2H), 7.43 (d, J=8.0 Hz, 2H), 4.64 (s, 1H), 4.61-4.53 (m, 2H), 4.52-4.49 (m, 1H), 4.37 (d, J=15.4 Hz, 1H), 4.13 (d, J=8.2 Hz, 2H), 3.92 (d, J=11.0 Hz, 1H), 3.82 (dd, J=10.9, 3.9 Hz, 1H), 3.10 (t, J=7.0 Hz, 2H), 2.49 (s, 3H), 2.37-2.05 (m, 12H), 1.70-1.57 (m, 4H), 1.51 (p, J=7.2 Hz, 2H), 1.42-1.32 (m, 3H), 1.05 (s, 9H), 0.98-0.86 (m, 2H). ESI m/z=720.4 [M+H]⁺.

((1R,8S,9s)-bicyclo[6.1.0]non-4-yn-9-yl)methyl (7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptyl)carbamate (VHLL-BCN #6)

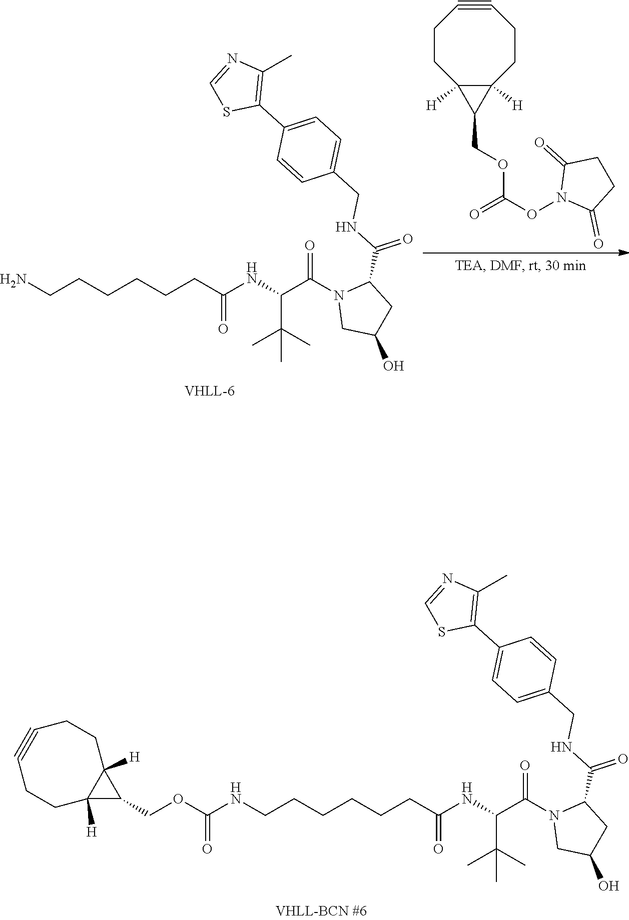 [SMILES: Cc1ncsc1-c1ccc(CNC(=O)[C@@H]2C[C@@H](O)CN2C(=O)[C@@H](NC(=O)CCCCCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCCCCCNC(=O)OC[C@@H]2[C@@H]3CCC#CCC[C@@H]32)C(C)(C)C)cc1]

VHLL-BCN #6 was synthesized following the standard procedure for preparing VHLL-BCN #1 from (2S,4R)-1-((S)-2-(7-aminoheptanamido)-3,3-dimethylbutanoyl)-4-hydroxy-N-(4-(4-methylthiazol-5-yl)benzyl)pyrrolidine-2-carboxamide (VHLL-6) and obtained as white solid (8.7 mg, 79%). ¹H NMR (600 MHZ, Methanol-d₄) δ 8.89 (s, 1H), 7.48 (d, J=7.9 Hz, 2H), 7.43 (d, J=8.1 Hz, 2H), 4.65 (s, 1H), 4.59 (t, J=8.3 Hz, 1H), 4.55 (d, J=15.6 Hz, 1H), 4.51 (s, 1H), 4.37 (d, J=15.3 Hz, 1H), 4.13 (d, J=8.1 Hz, 2H), 3.92 (d, J=11.0 Hz, 1H), 3.82 (dd, J=10.9, 3.9 Hz, 1H), 3.09 (t, J=7.0 Hz, 2H), 2.49 (s, 3H), 2.36-2.03 (m, 12H), 1.70-1.55 (m, 4H), 1.54-1.45 (m, 2H), 1.43-1.32 (m, 5H), 1.05 (s, 9H), 0.98-0.87 (m, 2H). ESI m/z=734.4 [M+H]⁺.

((1R,8S,9s)-bicyclo[6.1.0]non-4-yn-9-yl)methyl (8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctyl)carbamate (VHLL-BCN #7)

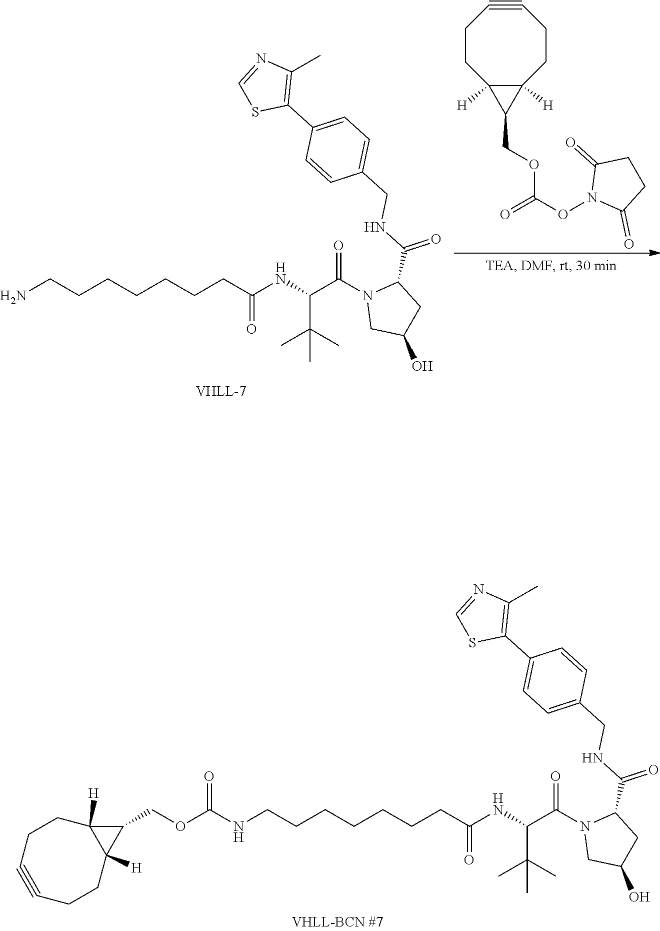 [SMILES: Cc1ncsc1-c1ccc(CNC(=O)[C@@H]2C[C@@H](O)CN2C(=O)[C@@H](NC(=O)CCCCCCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCCCCCCNC(=O)OC[C@@H]2[C@@H]3CCC#CCC[C@@H]32)C(C)(C)C)cc1]

VHLL-BCN #7 was synthesized following the standard procedure for preparing VHLL-BCN #1 from (2S,4R)-1-((S)-2-(8-aminooctanamido)-3,3-dimethylbutanoyl)-4-hydroxy-N-(4-(4-methylthiazol-5-yl)benzyl)pyrrolidine-2-carboxamide (VHLL-7) and obtained as white solid (8.4 mg, 75%). ¹H NMR (600 MHZ, Methanol-d₄) δ 8.89 (s, 1H), 7.48 (d, J=8.0 Hz, 2H), 7.43 (d, J=8.1 Hz, 2H), 4.65 (s, 1H), 4.62-4.53 (m, 2H), 4.53-4.49 (m, 1H), 4.37 (d, J=15.4 Hz, 1H), 4.13 (d, J=8.1 Hz, 2H), 3.92 (d, J=11.0 Hz, 1H), 3.82 (dd, J=10.9, 3.9 Hz, 1H), 3.09 (t, J=7.2 Hz, 2H), 2.49 (s, 3H), 2.34-2.07 (m, 12H), 1.68-1.56 (m, 4H), 1.52-1.44 (m, 2H), 1.43-1.32 (m, 7H), 1.05 (s, 9H), 0.98-0.87 (m, 2H). ESI m/z=748.5 [M+H]⁺.

((1R,8S,9s)-bicyclo[6.1.0]non-4-yn-9-yl)methyl (9-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-9-oxononyl)carbamate (VHLL-BCN #8)

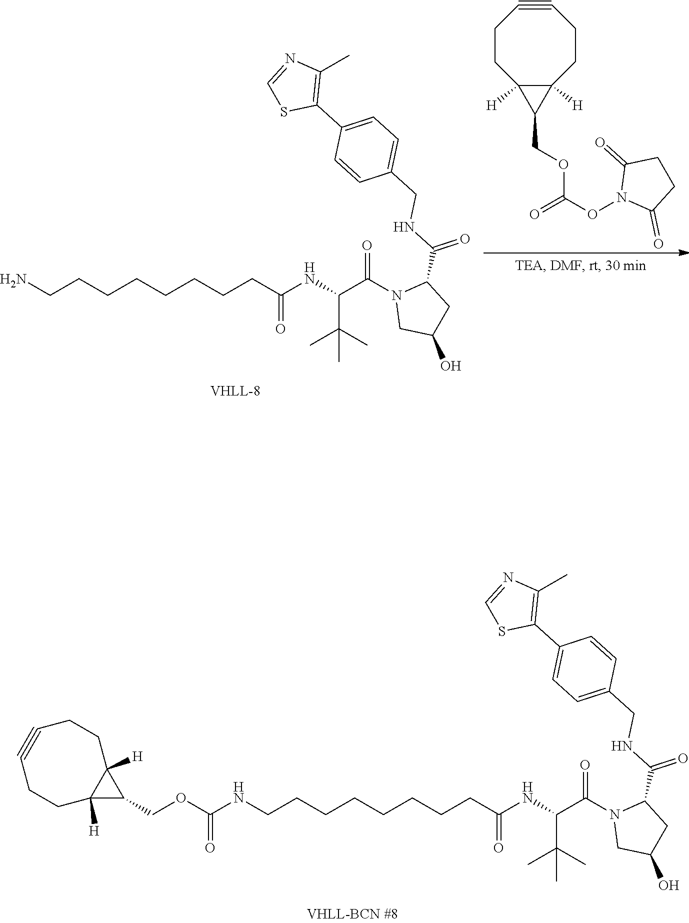 [SMILES: Cc1ncsc1-c1ccc(CNC(=O)[C@@H]2C[C@@H](O)CN2C(=O)[C@@H](NC(=O)CCCCCCCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCCCCCCCNC(=O)OC[C@@H]2[C@@H]3CCC#CCC[C@@H]32)C(C)(C)C)cc1]

VHLL-BCN #8 was synthesized following the standard procedure for preparing VHLL-BCN #1 from (2S,4R)-1-((S)-2-(9-aminononanamido)-3,3-dimethylbutanoyl)-4-hydroxy-N-(4-(4-methylthiazol-5-yl)benzyl)pyrrolidine-2-carboxamide (VHLL-8) and obtained as white solid (9.1 mg, 80%). ¹H NMR (600 MHZ, Methanol-d₄) δ 8.89 (s, 1H), 7.48 (d, J=8.2 Hz, 2H), 7.43 (d, J=8.2 Hz, 2H), 4.65 (s, 1H), 4.62-4.54 (m, 2H), 4.53-4.50 (m, 1H), 4.38 (d, J=15.5 Hz, 1H), 4.13 (d, J=7.9 Hz, 2H), 3.92 (d, J=11.0 Hz, 1H), 3.82 (dd, J=10.9, 3.9 Hz, 1H), 3.09 (t, J=6.9 Hz, 2H), 2.49 (s, 3H), 2.37-2.05 (m, 12H), 1.70-1.57 (m, 4H), 1.53-1.46 (m, 2H), 1.42-1.32 (m, 9H), 1.05 (d, J=2.5 Hz, 9H), 0.97-0.91 (m, 2H). ESI m/z=762.6 [M+H]⁺.

((1R,8S,9s)-bicyclo[6.1.0]non-4-yn-9-yl)methyl (10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecyl)carbamate (VHLL-BCN #9)

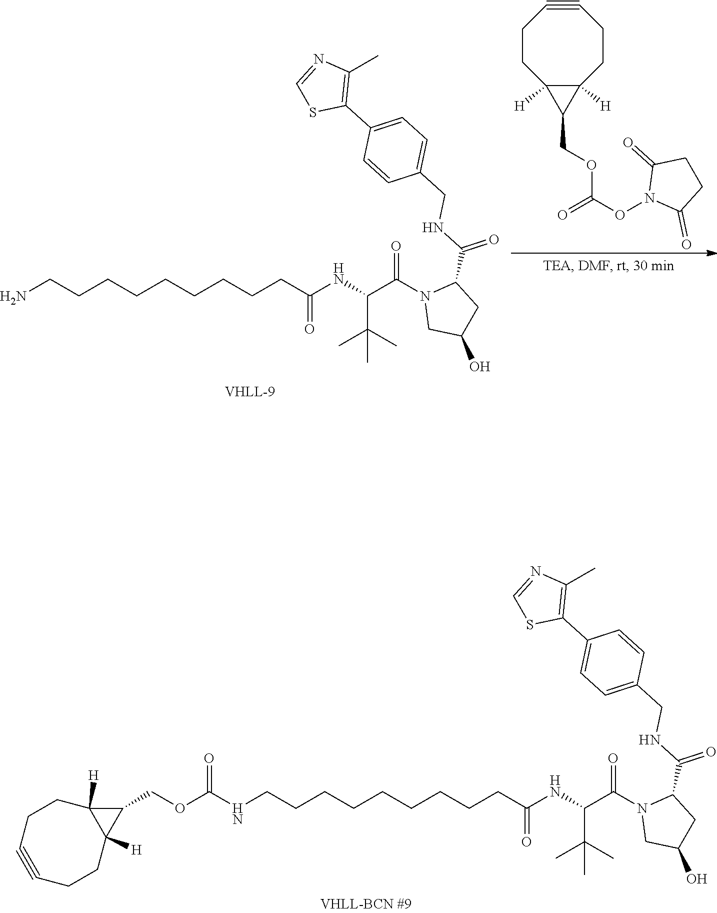 [SMILES: Cc1ncsc1-c1ccc(CNC(=O)[C@@H]2C[C@@H](O)CN2C(=O)[C@@H](NC(=O)CCCCCCCCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCCCCCCCCNC(=O)OC[C@@H]2[C@@H]3CCC#CCC[C@@H]32)C(C)(C)C)cc1]

VHLL-BCN #9 was synthesized following the standard procedure for preparing VHLL-BCN #1 from (2S,4R)-1-((S)-2-(10-aminodecanamido)-3,3-dimethylbutanoyl)-4-hydroxy-N-(4-(4-methylthiazol-5-yl)benzyl)pyrrolidine-2-carboxamide (VHLL-9) and obtained as white solid (7.9 mg, 68%). ¹H NMR (600 MHZ, Methanol-d₄) δ 8.89 (s, 1H), 7.48 (d, J=8.2 Hz, 2H), 7.43 (d, J=8.3 Hz, 2H), 4.65 (s, 1H), 4.62-4.53 (m, 2H), 4.53-4.49 (m, 1H), 4.37 (d, J=15.4 Hz, 1H), 4.14 (d, J=8.1 Hz, 2H), 3.92 (dt, J=11.1, 1.8 Hz, 1H), 3.82 (dd, J=11.0, 3.9 Hz, 1H), 3.09 (t, J=7.1 Hz, 2H), 2.49 (s, 3H), 2.36-2.13 (m, 11H), 2.10 (ddd, J=13.3, 9.0, 4.5 Hz, 1H), 1.62 (q, J=6.4, 5.8 Hz, 4H), 1.54-1.44 (m, 2H), 1.43-1.26 (m, 11H), 1.05 (s, 9H), 0.97-0.89 (m, 2H). ESI m/z=776.5 [M+H]⁺.

((1R,8S,9s)-bicyclo[6.1.0]non-4-yn-9-yl)methyl (11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecyl)carbamate (VHLL-BCN #10)

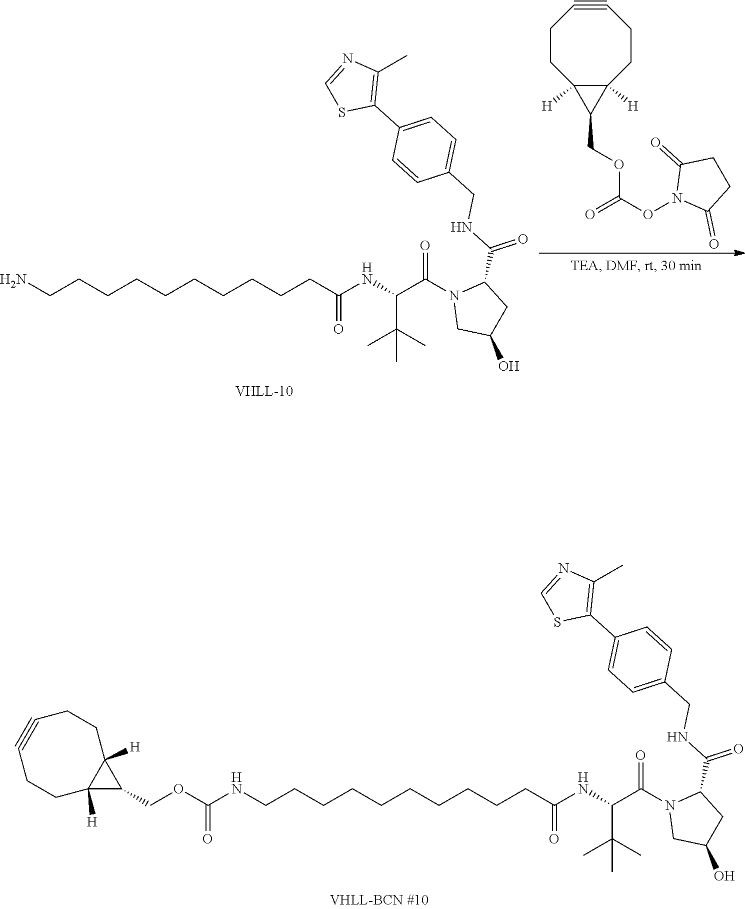 [SMILES: Cc1ncsc1-c1ccc(CNC(=O)[C@@H]2C[C@@H](O)CN2C(=O)[C@@H](NC(=O)CCCCCCCCCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCCCCCCCCCNC(=O)OC[C@@H]2[C@@H]3CCC#CCC[C@@H]32)C(C)(C)C)cc1]

VHLL-BCN #10 was synthesized following the standard procedure for preparing VHLL-BCN #1 from (2S,4R)-1-((S)-2-(11-aminoundecanamido)-3,3-dimethylbutanoyl)-4-hydroxy-N-(4-(4-methylthiazol-5-yl)benzyl)pyrrolidine-2-carboxamide (VHLL-10) and obtained as white solid (8.4 mg, 71%). ¹H NMR (600 MHZ, Methanol-d₄) δ 8.89 (s, 1H), 7.48 (d, J=8.1 Hz, 2H), 7.43 (d, J=8.2 Hz, 2H), 4.68-4.64 (m, 1H), 4.57 (s, 2H), 4.53-4.49 (m, 1H), 4.37 (d, J=15.5 Hz, 1H), 4.14 (d, J=8.1 Hz, 2H), 3.92 (d, J=11.0 Hz, 1H), 3.82 (dd, J=11.0, 3.9 Hz, 1H), 3.09 (t, J=7.0 Hz, 2H), 2.49 (s, 3H), 2.36-2.14 (m, 11H), 2.10 (ddd, J=13.3, 9.0, 4.5 Hz, 1H), 1.67-1.56 (m, 4H), 1.54-1.46 (m, 2H), 1.42-1.28 (m, 13H), 1.05 (s, 9H), 0.98 -0.89 (m, 2H). ESI m/z=790.5 [M+H]⁺.

((1R,8S,9s)-bicyclo[6.1.0]non-4-yn-9-yl)methyl (2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethyl)carbamate (VHLL-BCN #11)

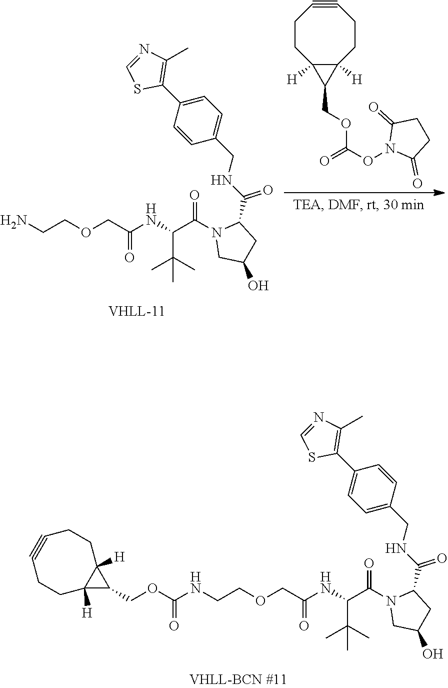 [SMILES: Cc1ncsc1-c1ccc(CNC(=O)[C@@H]2C[C@@H](O)CN2C(=O)[C@@H](NC(=O)COCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)COCCNC(=O)OC[C@@H]2[C@@H]3CCC#CCC[C@@H]32)C(C)(C)C)cc1]

VHLL-BCN #11 was synthesized following the standard procedure for preparing VHLL-BCN #1 from (2S,4R)-1-((S)-2-(2-(2-aminoethoxy)acetamido)-3,3-dimethylbutanoyl)-4-hydroxy-N-(4-(4-methylthiazol-5-yl)benzyl)pyrrolidine-2- carboxamide (VHLL-11) and obtained as white solid (7.9 mg, 74%). ¹H NMR (600 MHZ, Methanol-d₄) δ 8.89 (s, 1H), 7.49 (d, J=8.0 Hz, 2H), 7.45 (d, J=8.3 Hz, 2H), 4.72 (s, 1H), 4.65-4.57 (m, 2H), 4.52 (s, 1H), 4.35 (d, J=15.5 Hz, 1H), 4.14 (d, J=8.2 Hz, 2H), 4.06 (d, J=15.3 Hz, 1H), 4.01 (d, J=15.2 Hz, 1H), 3.90 (d, J=11.1 Hz, 1H), 3.83 (dd, J=11.0, 3.8 Hz, 1H), 3.66-3.57 (m, 2H), 3.43-3.34 (m, 2H), 2.50 (s, 3H), 2.30-2.02 (m, 10H), 1.66-1.54 (m, 2H), 1.40-1.34 (m, 1H), 1.06 (s, 9H), 0.95-0.86 (m, 2H). ESI m/z=708.3 [M+H]⁺.

((1R,8S,9s)-bicyclo[6.1.0]non-4-yn-9-yl)methyl (2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl)carbamate (VHLL-BCN #12)

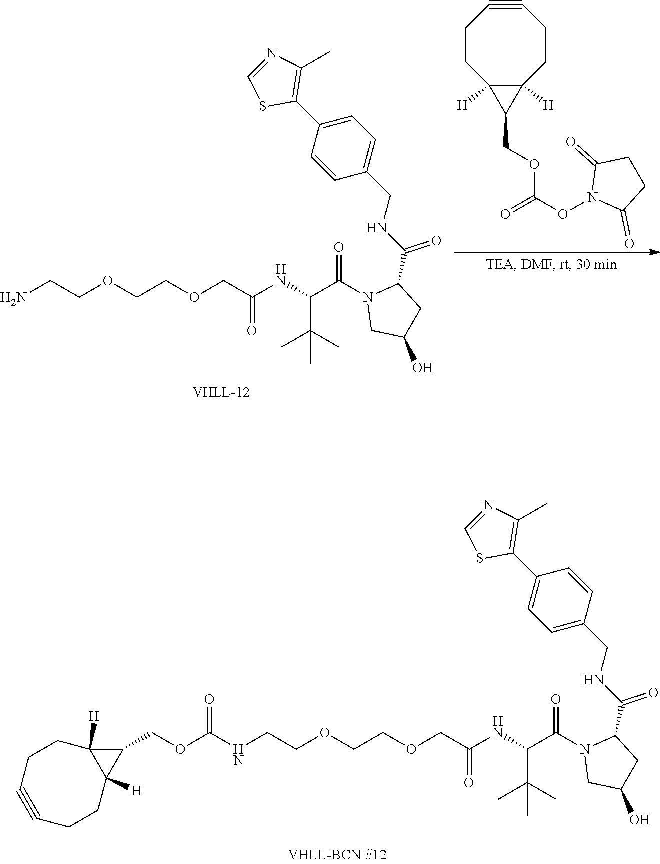 [SMILES: Cc1ncsc1-c1ccc(CNC(=O)[C@@H]2C[C@@H](O)CN2C(=O)[C@@H](NC(=O)COCCOCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)COCCOCCNC(=O)OC[C@@H]2[C@@H]3CCC#CCC[C@@H]32)C(C)(C)C)cc1]

VHLL-BCN #12 was synthesized following the standard procedure for preparing VHLL-BCN #1 from (2S,4R)-1-((S)-2-(2-(2-(2-aminoethoxy)ethoxy)acetamido)-3,3-dimethylbutanoyl)-4-hydroxy-N-(4-(4-methylthiazol-5-yl)benzyl)pyrrolidine-2-carboxamide (VHLL-12) and obtained as white solid (8.1 mg, 72%). ¹H NMR (600 MHZ, Methanol-d₄) 8 8.89 (s, 1H), 7.47 (d, J=6.1 Hz, 2H), 7.44 (d, J=8.3 Hz, 2H), 4.74 (s, 1H), 4.63-4.54 (m, 2H), 4.53 (s, 1H), 4.36 (d, J=15.4 Hz, 1H), 4.11 (d, J=8.1 Hz, 2H), 4.08 (d, J=15.9 Hz, 1H), 4.03 (d, J=16.0 Hz, 1H), 3.88 (d, J=11.1 Hz, 1H), 3.82 (dd, J=11.0, 3.8 Hz, 1H), 3.76-3.70 (m, 2H), 3.69-3.64 (m, 2H), 3.60-3.53 (m, 2H), 3.37-3.25 (m, 2H), 2.50 (s, 3H), 2.31-2.08 (m, 10H), 1.66-1.52 (m, 2H), 1.38-1.33 (m, 1H), 1.06 (s, 9H), 0.97-0.85 (m, 2H). ESI m/z =752.4 [M+H]⁺.

((1R,8S,9s)-bicyclo[6.1.0]non-4-yn-9-yl)methyl ((S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl)carbamate (VHLL-BCN #13)

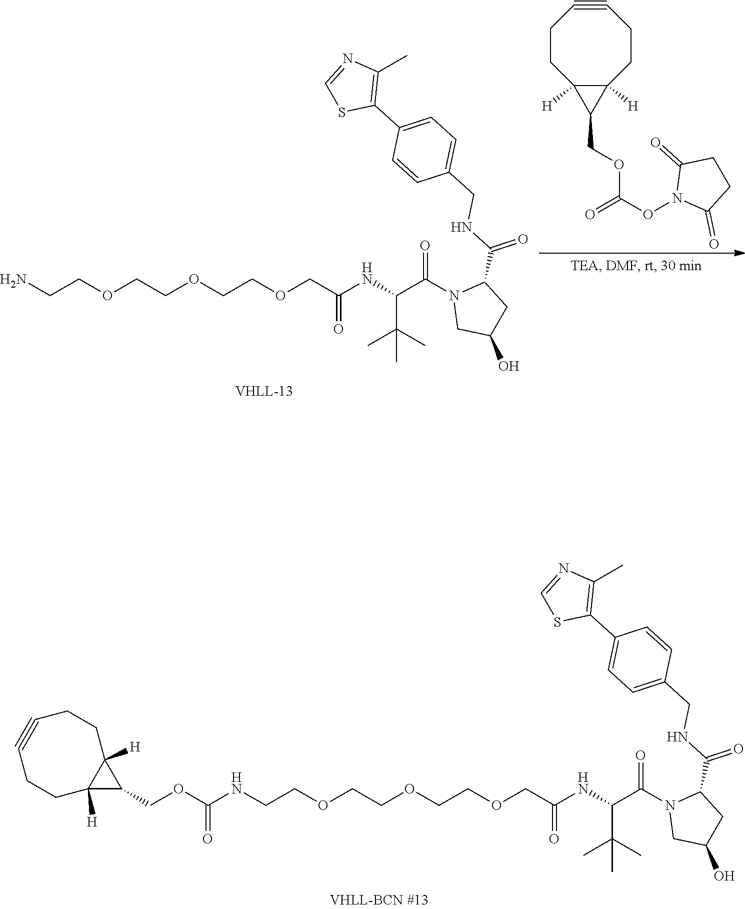 [SMILES: Cc1ncsc1-c1ccc(CNC(=O)[C@@H]2C[C@@H](O)CN2C(=O)[C@@H](NC(=O)COCCOCCOCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)COCCOCCOCCNC(=O)OC[C@@H]2[C@@H]3CCC#CCC[C@@H]32)C(C)(C)C)cc1]

VHLL-BCN #13 was synthesized following the standard procedure for preparing VHLL-BCN #1 from (2S,4R)-1-((S)-14-amino-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide (VHLL-13) and obtained as white solid (8.5 mg, 71%). ¹H NMR (600 MHZ, Methanol-d₄) 8 8.90 (s, 1H), 7.48 (d, J=8.0 Hz, 2H), 7.44 (d, J=8.2 Hz, 2H), 4.73 (d, J=9.4 Hz, 1H), 4.64 -4.54 (m, 2H), 4.52 (dt, J=4.5, 2.3 Hz, 1H), 4.38 (d, J=15.4 Hz, 1H), 4.14 (d, J=8.0 Hz, 2H), 4.08 (d, J=5.9 Hz, 2H), 3.89 (d, J=11.0 Hz, 1H), 3.83 (dd, J=11.0, 3.8 Hz, 1H), 3.76-3.61 (m, 8H), 3.52 (t, J=5.5 Hz, 2H), 3.28 (q, J=5.6 Hz, 2H), 2.50 (s, 3H), 2.30-2.04 (m, 10H), 1.65-1.56 (m, 2H), 1.41-1.33 (m, 1H), 1.06 (s, 9H), 0.97-0.91 (m, 2H). ESI m/z=796.5 [M+H]⁺.

((1R,8S,9s)-bicyclo[6.1.0]non-4-yn-9-yl)methyl (2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethyl)carbamate (VHLL-BCN #14)

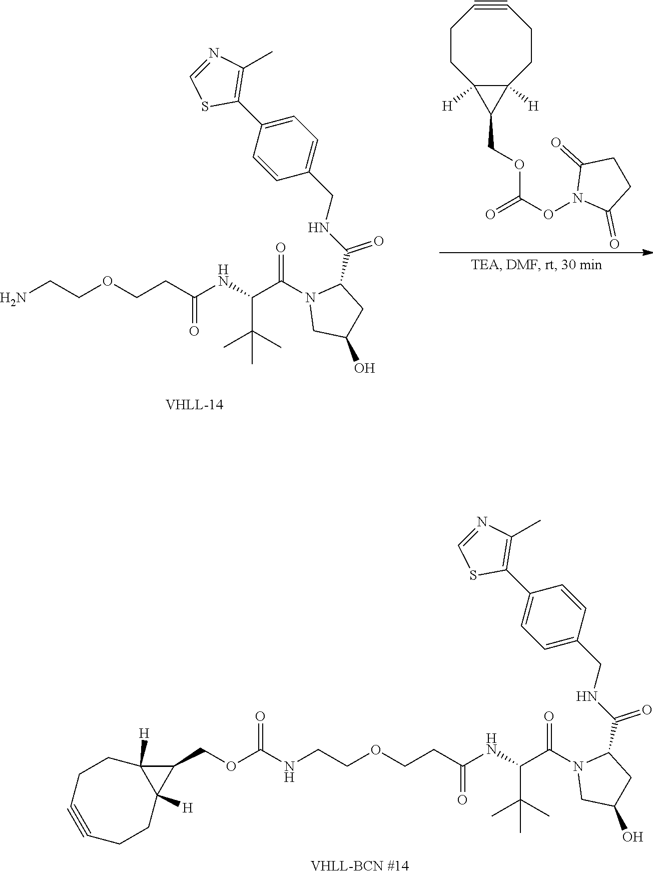 [SMILES: Cc1ncsc1-c1ccc(CNC(=O)[C@@H]2C[C@@H](O)CN2C(=O)[C@@H](NC(=O)CCOCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCOCCNC(=O)OC[C@H]2[C@@H]3CCC#CCC[C@@H]32)C(C)(C)C)cc1]

VHLL-BCN #14 was synthesized following the standard procedure for preparing VHLL-BCN #1 from (2S,4R)-1-((S)-2-(3-(2-aminoethoxy)propanamido)-3,3-dimethylbutanoyl)-4-hydroxy-N-(4-(4-methylthiazol-5-yl)benzyl)pyrrolidine-2-carboxamide (VHLL-14) and obtained as white solid (9.2 mg, 85%). ¹H NMR (600 MHZ, Methanol-d₄) δ 8.89 (s, 1H), 7.48 (d, J=7.9 Hz, 2H), 7.43 (d, J=7.8 Hz, 2H), 4.68 (d, J=8.9 Hz, 1H), 4.63-4.56 (m, 2H), 4.52 (s, 1H), 4.35 (d, J=15.5 Hz, 1H), 4.12 (d, J=7.9 Hz, 2H), 3.91 (d, J=11.1 Hz, 1H), 3.82 (dd, J=11.0, 3.9 Hz, 1H), 3.75-3.67 (m, 2H), 3.57-3.50 (m, 2H), 3.34-3.28 (m, 2H), 2.60-2.45 (m, 5H), 2.32-2.01 (m, 10H), 1.67-1.55 (m, 2H), 1.44-1.31 (m, 1H), 1.06 (s, 9H), 0.97-0.88 (m, 2H). ESI m/z=722.4 [M+H]⁺.

((1R,8S,9s)-bicyclo[6.1.0]non-4-yn-9-yl)methyl (2-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)ethyl)carbamate (VHLL-BCN #15)

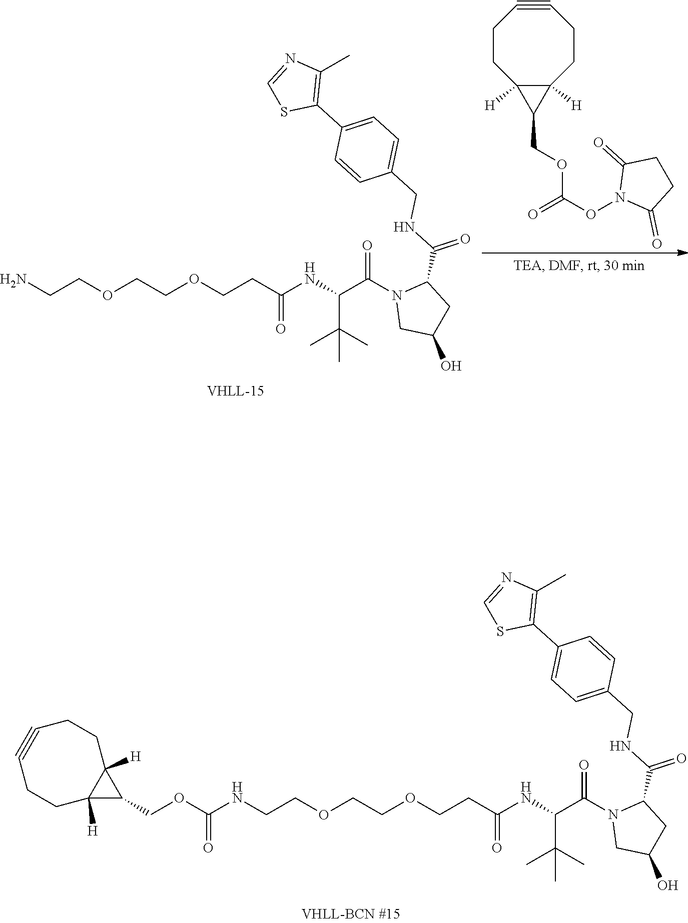 [SMILES: Cc1ncsc1-c1ccc(CNC(=O)[C@@H]2C[C@@H](O)CN2C(=O)[C@@H](NC(=O)CCOCCOCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCOCCOCCNC(=O)OC[C@@H]2[C@@H]3CCC#CCC[C@@H]32)C(C)(C)C)cc1]

VHLL-BCN #15 was synthesized following the standard procedure for preparing VHLL-BCN #1 from (2S,4R)-1-((S)-2-(3-(2-(2-aminoethoxy)ethoxy)propanamido)-3,3-dimethylbutanoyl)-4-hydroxy-N-(4-(4-methylthiazol-5-yl)benzyl)pyrrolidine-2-carboxamide (VHLL-15) and obtained as white solid (7.7 mg, 67%). ¹H NMR (600 MHZ, Methanol-d₄) δ 8.90 (s, 1H), 7.49 (d, J=8.0 Hz, 2H), 7.44 (d, J=8.1 Hz, 2H), 4.69 (s, 1H), 4.62-4.54 (m, 2H), 4.52 (s, 1H), 4.38 (d, J=15.4 Hz, 1H), 4.14 (d, J=8.1 Hz, 2H), 3.91 (d, J=11.0 Hz, 1H), 3.83 (dd, J=11.0, 3.9 Hz, 1H), 3.80-3.71 (m, 2H), 3.70-3.60 (m, 4H), 3.54 (t, J=5.6 Hz, 2H), 3.29 (t, J=5.5 Hz, 2H), 2.66-2.56 (m, 1H), 2.54-2.47 (m, 4H), 2.31-2.03 (m, 10H), 1.66 -1.54 (m, 2H), 1.43-1.33 (m, 1H), 1.06 (s, 9H), 0.99-0.90 (m, 2H). 13C NMR (151 MHZ, Methanol-d₄) δ 174.42, 173.76, 172.11, 159.19, 152.83, 149.03, 140.26, 133.38, 131.50, 130.34, 128.97, 99.53, 71.45, 71.23, 71.05, 71.03, 68.28, 63.69, 60.78, 58.93, 57.99, 43.70, 41.66, 38.90, 37.38, 36.79, 30.13, 27.03, 21.93, 21.36, 18.94, 15.87. HRMS (ESI-TOF) calcd for C₄₀H₅₆N₅O₈S⁺ [M+H]⁺ 766.3844, found 766.3838. HPLC>97%, t_{R}=4.53 min.

((1R,8S,9s)-bicyclo[6.1.0]non-4-yn-9-yl)methyl ((S)-14-((2S,4R)-4-hydroxy-2-((4-(4-methylthiazol-5-yl)benzyl)carbamoyl)pyrrolidine-1-carbonyl)-15,15-dimethyl-12-oxo-3,6,9-trioxa-13-azahexadecyl)carbamate (VHLL-BCN #16)

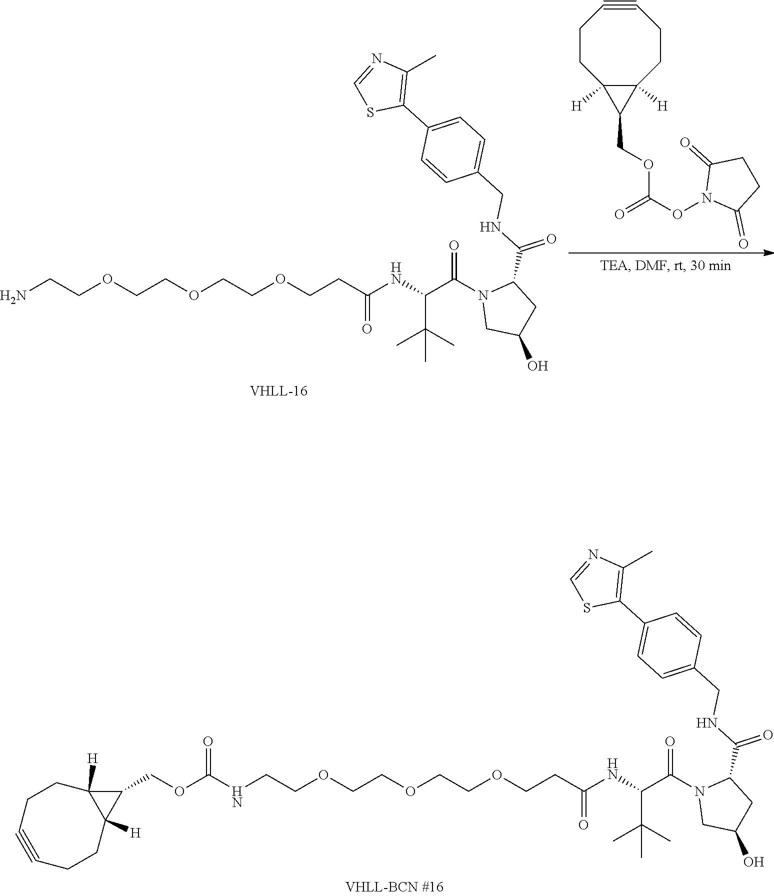 [SMILES: Cc1ncsc1-c1ccc(CNC(=O)[C@@H]2C[C@@H](O)CN2C(=O)[C@@H](NC(=O)CCOCCOCCOCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCOCCOCCOCCNC(=O)OC[C@@H]2[C@@H]3CCC#CCC[C@@H]32)C(C)(C)C)cc1]

VHLL-BCN #16 was synthesized following the standard procedure for preparing VHLL-BCN #1 from (2S,4R)-1-((S)-1-amino-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (VHLL-16) and obtained as white solid (9.4 mg, 77%). ¹H NMR (600 MHZ, Methanol-d₄) δ 8.90 (s, 1H), 7.49 (d, J=8.0 Hz, 2H), 7.44 (d, J=8.2 Hz, 2H), 4.69-4.66 (m, 1H), 4.62-4.55 (m, 2H), 4.54-4.50 (m, 1H), 4.38 (d, J=15.4 Hz, 1H), 4.15 (d, J=8.1 Hz, 2H), 3.91 (d, J=10.9 Hz, 1H), 3.83 (dd, J=11.0, 3.9 Hz, 1H), 3.81-3.71 (m, 2H), 3.68-3.60 (m, 8H), 3.53 (t, J=5.5 Hz, 2H), 3.29 (t, J=5.5 Hz, 2H), 2.60 (ddd, J=14.8, 7.5, 5.2 Hz, 1H), 2.54-2.47 (m, 4H), 2.32-2.04 (m, 10H), 1.69-1.56 (m, 2H), 1.43-1.35 (m, 1H), 1.06 (s, 9H), 1.00-0.89 (m, 2H). ¹³C NMR (151 MHZ, Methanol-d₄) δ 174.42, 173.74, 172.10, 159.18, 152.83, 149.02, 140.27, 133.37, 131.48, 130.34, 128.97, 99.55, 71.58, 71.52, 71.38, 71.25, 71.05, 70.97, 68.29, 63.67, 60.78, 58.96, 57.98, 43.68, 41.67, 38.90, 37.40, 36.78, 30.13, 27.04, 21.95, 21.36, 18.94, 15.88. HRMS (ESI-TOF) calcd for C₄₂H₆₀N₅O₉S⁺ [M+H]⁺ 810.4106, found 810.4102. HPLC>98%, t_{R}=4.51 min.

(25,4S)-1-((S)-1-amino-14-(tert-butyl)-12-oxo-3,6,9-trioxa-13-azapentadecan-15-oyl)-4-hydroxy-N-(4-(4-methylthiazol-5-yl)benzyl)pyrrolidine-2- carboxamide (VHLL-16-NC)

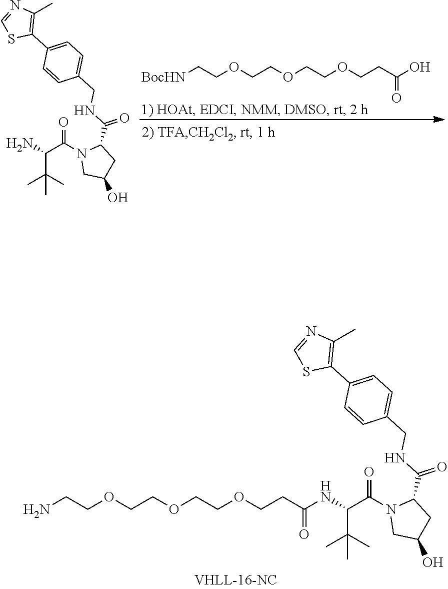 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCOCCOCCOCCC(=O)O ; Cc1ncsc1-c1ccc(CNC(=O)[C@@H]2C[C@@H](O)CN2C(=O)[C@@H](NC(=O)CCOCCOCCOCCN)C(C)(C)C)cc1]

To a solution of (25,4S)-1-((S)-2-amino-3,3-dimethylbutanoyl)-4-hydroxy-N-(4-(4-methylthiazol-5-yl)benzyl)pyrrolidine-2-carboxamide HCI salt (51.4 mg, 0.11 mmol, 1.1 equiv) in DMSO (2 mL) were added compound 2,2-dimethyl-4-oxo-3,8,11,14-tetraoxa-5-azaheptadecan-17-oic acid (32.1 mg, 0.1 mmol, 1.0 equiv), EDCI (1-ethyl-3-(3-dimethylaminopropyl)carbodiimide) (38.4 mg, 0.2 mmol, 2.0 equiv), HOAt (1-hydroxy-7-azabenzo- triazole) (27.2 mg, 0.2 mmol, 2.0 equiv) and NMM (N-Methylmorpholine) (40.4 mg, 0.4 mmol, 4.0 equiv). After being stirred at room temperature for 2 h, the resulting mixture was purified by preparative HPLC (10%-100% acetonitrile/0.1% TFA in H₂O) to afford colorless oil. The obtained oil was dissolved in CH₂Cl2 (dichloromethane) (1 mL) and TFA (trifluoroacetic acid) (1 mL) was added at room temperature. After being stirred at room temperature for 1 h, the resulting mixture was concentrated and purified by preparative HPLC (10%-100% acetonitrile/0.1% TFA in H₂O) to afford the VHLL-16-NC as colorless oil in TFA salt form (25.0 mg, 33% for two steps). ¹H NMR (600 MHZ, Methanol-d₄) δ 9.13 (s, 1H), 7.38 (d, J=8.2 Hz, 2H), 7.34 (d, J=8.2 Hz, 2H), 4.49-4.36 (m, 3H), 4.34-4.27 (m, 2H), 3.92 (dd, J=10.6, 5.1 Hz, 1H), 3.68-3.48 (m, 13H), 3.02 (t, J=5.0 Hz, 2H), 2.52-2.45 (m, 1H), 2.43-2.36 (m, 4H), 2.34 (ddd, J=13.3, 9.1, 5.3 Hz, 1H), 1.89 (dt, J=13.3, 4.5 Hz, 1H), 0.94 (s, 9H). ESI m/z=634.4 [M +H]⁺.

((1R,8S,9s)-bicyclo[6.1.0]non-4-yn-9-yl)methyl ((S)-14-((2S,4S)-4-hydroxy-2-((4-(4-methylthiazol-5-yl)benzyl)carbamoyl)pyrrolidine-1-carbonyl)-15,15-dimethyl-12-oxo-3,6,9-trioxa-13-azahexadecyl)carbamate (VHLL-BCN #16-NC)

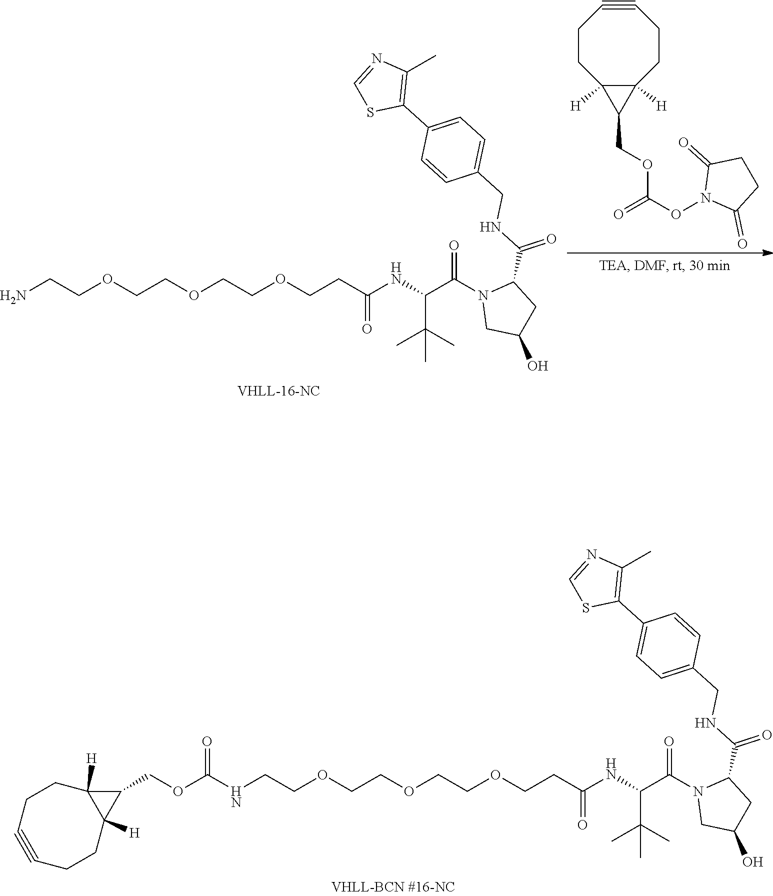 [SMILES: Cc1ncsc1-c1ccc(CNC(=O)[C@@H]2C[C@@H](O)CN2C(=O)[C@@H](NC(=O)CCOCCOCCOCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCOCCOCCOCCNC(=O)OC[C@@H]2[C@@H]3CCC#CCC[C@@H]32)C(C)(C)C)cc1]

VHLL-BCN #16-NC was synthesized following the standard procedure for preparing VHLL-BCN #1 from (2S,4S)-1-((S)-1-amino-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (VHLL-16-NC) and obtained as white solid (7.8 mg, 64%). ¹H NMR (600 MHZ, Methanol-d₄) δ 8.90 (s, 1H), 7.48 (d, J=8.0 Hz, 2H), 7.44 (d, J=7.9 Hz, 2H), 4.62-4.50 (m, 3H), 4.45-4.36 (m, 2H), 4.15 (d, J=8.1 Hz, 2H), 4.05 (dd, J=10.6, 5.1 Hz, 1H), 3.79-3.71 (m, 3H), 3.68 -3.59 (m, 8H), 3.54 (t, J=5.5 Hz, 2H), 3.29 (t, J=5.6 Hz, 2H), 2.67-2.56 (m, 1H), 2.54-2.42 (m, 5H), 2.34-2.12 (m, 8H), 2.04-1.98 (m, 1H), 1.68-1.56 (m, 2H), 1.43-1.35 (m, 1H), 1.06 (s, 9H), 0.99-0.89 (m, 2H). ESI m/z=832.5 [M+Na]⁺. HRMS (ESI-TOF) calcd for C₄₂H₆₀N₅O₉S⁺ [M+H]⁺ 810.4106, found 810.4111.

((1R,8S,9s)-bicyclo[6.1.0]non-4-yn-9-yl)methyl ((S)-17-((2S,4R)-4-hydroxy-2-((4-(4-methylthiazol-5-yl)benzyl)carbamoyl)pyrrolidine-1-carbonyl)-18,18-dimethyl-15-oxo-3,6,9,12-tetraoxa-16-azanonadecyl)carbamate (VHLL-BCN #17)

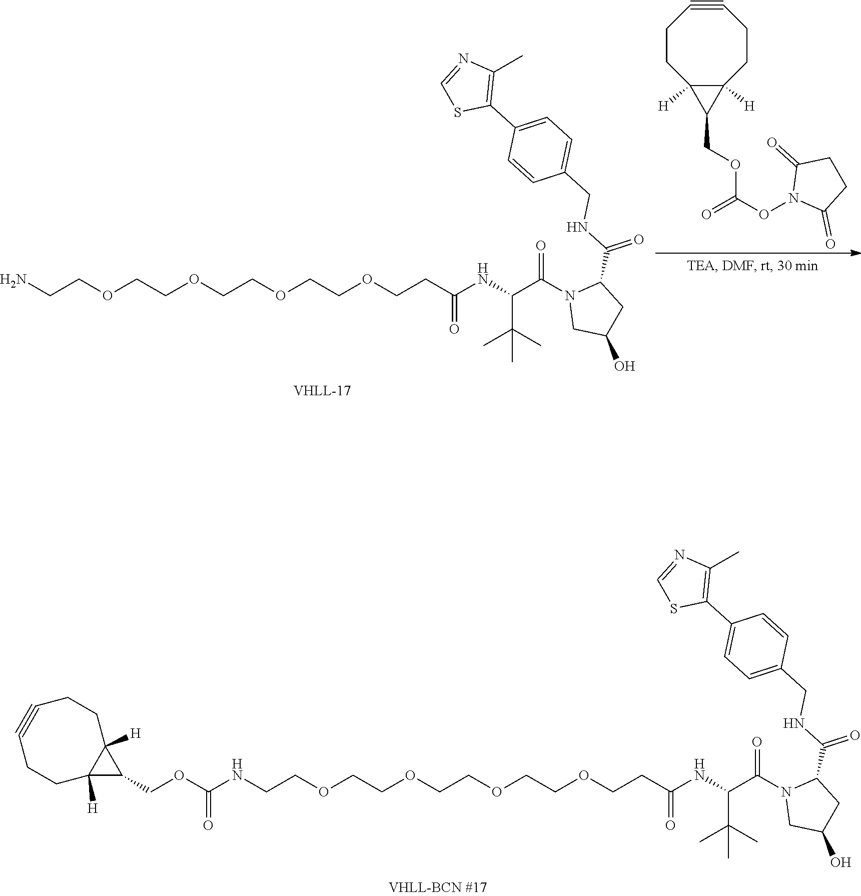 [SMILES: Cc1ncsc1-c1ccc(CNC(=O)[C@@H]2C[C@@H](O)CN2C(=O)[C@@H](NC(=O)CCOCCOCCOCCOCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCOCCOCCOCCOCCNC(=O)OC[C@@H]2[C@@H]3CCC#CCC[C@@H]32)C(C)(C)C)cc1]

VHLL-BCN #17 was synthesized following the standard procedure for preparing VHLL-BCN #1 from (2S,4R)-1-((S)-1-amino-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (VHLL-17) and obtained as white solid (9.7 mg, 76%). ¹H NMR (600 MHZ, Methanol-d₄) δ 8.91 (s, 1H), 7.50 (d, J=8.2 Hz, 2H), 7.44 (d, J=8.2 Hz, 2H), 4.69 (s, 1H), 4.64-4.56 (m, 2H), 4.53 (s, 1H), 4.39 (d, J=15.6 Hz, 1H), 4.16 (d, J=8.2 Hz, 2H), 3.92 (d, J=11.0 Hz, 1H), 3.83 (dd, J=10.9, 3.9 Hz, 1H), 3.80-3.72 (m, 2H), 3.69-3.60 (m, 12H), 3.55 (t, J=5.6 Hz, 2H), 3.30 (t, J=5.6 Hz, 2H), 2.61 (ddd, J=15.1, 7.5, 5.2 Hz, 1H), 2.54-2.48 (m, 4H), 2.30-2.15 (m, 9H), 2.12 (ddd, J=13.2, 9.1, 4.5 Hz, 1H), 1.66-1.57 (m, 2H), 1.43-1.36 (m, 1H), 1.07 (s, 9H), 0.99-0.90 (m, 2H). ¹³C NMR (151 MHZ, Methanol-d₄) δ 174.43, 173.67, 172.08, 159.19, 152.84, 149.03, 140.28, 133.38, 131.48, 130.34, 128.97, 99.55, 71.55, 71.50, 71.41, 71.26, 71.05, 70.99, 68.28, 63.67, 60.78, 58.87, 57.98, 43.68, 41.68, 38.91, 37.36, 36.78, 30.14, 27.04, 21.95, 21.36, 18.95, 15.88. ESI m/z=854.5 [M+H]⁺. HRMS (ESI-TOF) calcd for C₄₄H₆₄N₅O₁₀S⁺ [M+H]⁺ 854.4368, found 854.4360. HPLC>98%, t_{R}=4.53 min.

((1R,8S,9s)-bicyclo[6.1.0]non-4-yn-9-yl)methyl ((S)-20-((2S,4R)-4-hydroxy-2-((4-(4-methylthiazol-5-yl)benzyl)carbamoyl)pyrrolidine-1-carbonyl)-21,21-dimethyl-18-oxo-3,6,9,12, 15-pentaoxa-19-azadocosyl)carbamate (VHLL-BCN #18)

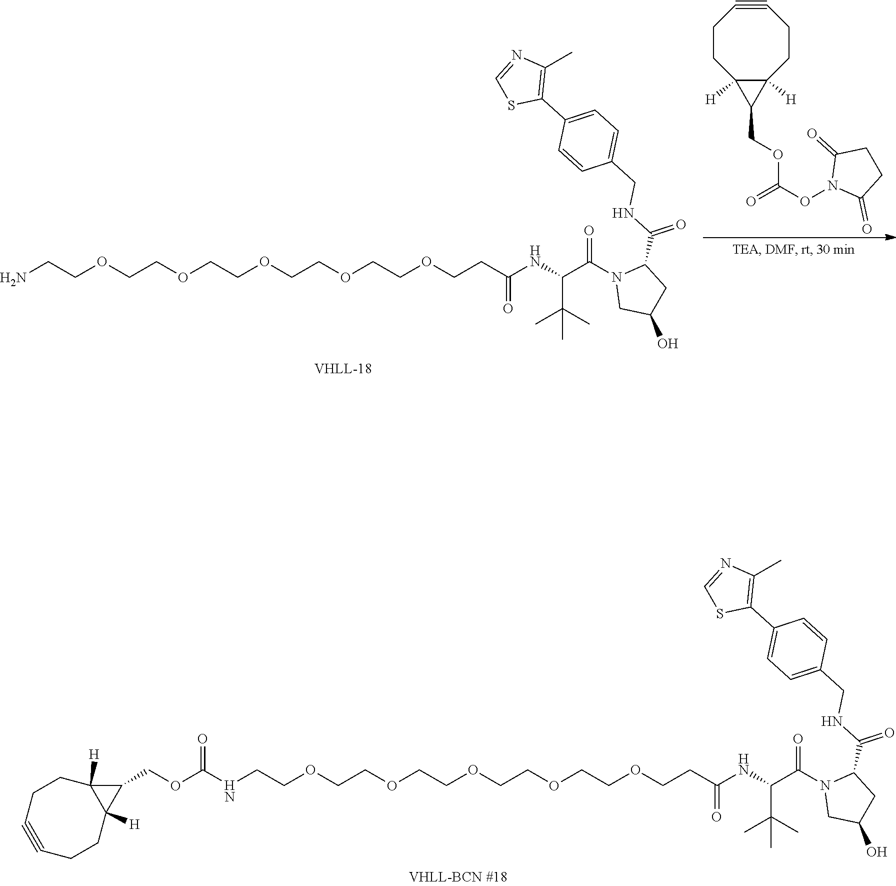 [SMILES: Cc1ncsc1-c1ccc(CNC(=O)[C@@H]2C[C@@H](O)CN2C(=O)[C@@H](NC(=O)CCOCCOCCOCCOCCOCCN)C(C)(C)C)cc1 ; O=C(OC[C@@H]1[C@@H]2CCC#CCC[C@@H]21)ON1C(=O)CCC1=O ; Cc1ncsc1-c1ccc(CNC(=O)[C@@H]2C[C@@H](O)CN2C(=O)[C@@H](NC(=O)CCOCCOCCOCCOCCOCCNC(=O)OC[C@@H]2[C@@H]3CCC#CCC[C@@H]32)C(C)(C)C)cc1]

VHLL-BCN #18 was synthesized following the standard procedure for preparing VHLL-BCN #1 from (2S,4R)-1-((S)-1-amino-20-(tert-butyl)-18-oxo-3,6,9, 12, 15-pentaoxa-19-azahenicosan-21-oyl)-4-hydroxy-N-(4-(4-methylthiazol-5-yl)benzyl)pyrrolidine-2-carboxamide (VHLL-18) and obtained as white solid (9.5 mg, 71%). ¹H NMR (600 MHZ, Methanol-d₄) δ 8.90 (s, 1H), 7.49 (d, J=8.2 Hz, 2H), 7.44 (d, J=8.2 Hz, 2H), 4.67 (s, 1H), 4.62-4.55 (m, 2H), 4.53-4.49 (m, 1H), 4.38 (d, J=15.5 Hz, 1H), 4.15 (d, J=8.1 Hz, 2H), 3.91 (d, J=10.9 Hz, 1H), 3.82 (dd, J=10.9, 3.9 Hz, 1H), 3.79-3.72 (m, 2H), 3.69-3.59 (m, 16H), 3.54 (t, J=5.5 Hz, 2H), 3.29 (t, J=5.5 Hz, 2H), 2.60 (ddd, J=15.0, 7.5, 5.2 Hz, 1H), 2.53-2.46 (m, 4H), 2.29 -2.14 (m, 9H), 2.10 (ddd, J=13.3, 9.1, 4.5 Hz, 1H), 1.67-1.56 (m, 2H), 1.44-1.33 (m, 1H), 1.06 (s, 9H), 0.98-0.90 (m, 2H). ESI m/z=898.5 [M+H]⁺.

### Experimental Methods: Oligomer synthesis

The single strand oligonucleotides containing the NF-κB binding motif, namely NF-κB-ODN, was synthesized based on the HIV KB site. The sequence of 31-mer NF-κB-ODN is 5′-TGGGGACTTTCCAGTTTCTGGAAAGTCCCCA-3′ (SEQ ID NO: 4), in which the KB site is underlined. Moreover, the azide modification version of NF-κB-ODN, namely N3-NF-κB-ODN, was further synthesized by incorporating an azide group on the 5′ end of NF-κB-ODN through the 5′ amino modifier C_{6.} Similarly, the Biotin-NF-κB-ODN was synthesized by adding a biotin to the 5′-end of NF-κB-ODN using a spacer of NH2—(CH₂)₂—O—(CH₂)₂—OH.

The double-stained 15-mers oligonucleotide, namely E2F-ODN, contains the E2F binding motif derived from MYC promoter. The sense chain is 5′-CTAGATTTCCCGCG-3′ (SEQ ID NO: 5) and the antisense chain is 5′-CTAGCGCGGAAAT-3′ (SEQ ID NO: 7), in which the E2F binding site is underlined. Furthermore, N3-E2F-ODN and Biotin-E2F-ODN were synthesized by adding the azide group or biotin on the 5′-end of the sense stain oligomer.

All oligomers were synthesized by Integrated DNA Technologies, Inc. The unmodified oligomers were purified by standard desalting, while both azide- and biotin-modified oligomers were purified by HPLC. The single NF-κB-ODN (unmodified and modified) were annealed by heating the solution to 95° C. for 5 min, followed by cooled down to room temperature in 5° C. per min. Similarly, the sense and antisense E2F oligomers were mixed at 1:1 ratio and annealed before use.

### Experimental Methods: In Vitro Copper-Free Strain-Promoted Azide-Alkyne Cycloaddition (SPAAC) Reaction

For incorporation of VHL ligand onto the oligomers, BCNmodified VHL ligands (VHLL-X-BCN) were incubated with either N3-NF-κB-ODN or the azide-modified sense stain of E2F oligomer in phosphate buffered saline (PBS) buffer at 37° C. for indicated time periods. The reaction mixtures were further purified by the Nucleotide Removal Kit (QIAGEN), followed by annealing as described above.

### Experimental Methods: Native DNA Polyacrylamide Gel Electrophoresis (PAGE)

Oligomers were separated by 20% native polyacrylamide gel electrophoresis (PAGE) at 100 V for 1 hour, followed by incubated in 0.2% EtBr solution in 1 X Tris-boric acid-EDTA (TBE, pH 8.3) buffer that consisted of 89 mM Tris, 89 mM boric acid, 2 mM EDTA. The native gels contain 20% acrylamide and 2.5% glycerol, 0.075 ammonium persulfate (APS), 0.05% tetramethylethylenediamine (TEMED) in 0.5 X Tris-boric acid-EDTA (TBE) buffer. Finally, the gels were imaged with UV illumination with the ChemiDoc™M Touch Imaging System (Bio-Rad).

### Experimental Methods: Cell Culture and Treatment

Human embryonic kidney 293T (HEK293T) and HeLa cells were maintained in Dulbecco's Modified Eagle's Medium (DMEM) containing 10% fetal bovine serum (FBS), 100 Units/mL of penicillin and 100 μg/mL streptomycin. For ectopic expression of transcription factors, Flag-p65 and HA-E2F1 were transfected into HEK293T cells and harvested for lysis 48 hours after transfection. For TF-PROTACs treatment, HeLa cells in 12-well plate were transfected with individual TF-PROTAC for 12 hours, followed by harvest for western blot analysis. For proteasome or VHL ligand inhibition assay, cells were treated with respective TF-PROTACs, together with either 30 μM of MG132 (BML-P1102, ENZO Life Sciences) or 10 μM of VHL ligand (VH-032) for 12 hours. For depletion of endogenous VHL E3 ligase, two sgRNA for VHL E3 ligase were synthesized and inserted into lenti-CRISPR-V2 construct 40. The sgVHL lentivirus were generated in HEK293T cells as previously described 41-42 for infection of HeLa cells overnight, followed by selection with puromycin for 72 h.

### Experimental Methods: Streptavidin-Biotin Pulldown Assay

HEK293T cells that ectopically expressed either Flag-p65 or HAE2F1 were lysed by EBC buffer (50 mM Tris pH 7.5, 120 mM NaCl, 0.5% NP-40) supplemented with protease inhibitors (Pierce) and phosphatase inhibitors (phosphatase inhibitor cocktail set I and II, Calbiochem). The protein concentrations of the lysates were measured using the Bio-Rad protein assay reagent on a Beckman Coulter DU-800 spectrophotometer. The cell lysates (1 mg) were further incubated with biotin-ODN at 4ºC for 3 hours, followed by adding 10 μL of Streptavidin agarose beads (Thermo Fisher) for another 1 hour. The beads were washed 4 times with NETN buffer (100 mM NaCl, 20 mM Tris-Cl, pH 8.0, 0.5 mM EDTA, 0.5% NP-40), boiled in SDS loading buffer and further separated by 10% SDS-PAGE and blotted with individual antibody.

### Experimental Methods: In Vivo Ubiquitination Assay

Denatured in vivo ubiquitination assays were performed as previously described 41. Briefly, HEK293T cells were firstly transfected with Flag-p65 or HA-E2F1 for 24 hours, followed by additional transfection of individual TF-PROTAC for another 12 hours. Before been harvested, HEK293T cells were treated with 30 μM of MG132 for 4 hours. After having been harvested in denatured buffer A (6 M guanidine-HCI, pH 8.0, 0.1 M Na₂HPO₄/NaH₂PO₄, and 10 mM imidazole) and sonication for 10 seconds, cell lysis was incubation with Ni-nitrilotriacetic acid (NTA) matrices under rotation for 3 hours at room temperature. The pulldown products were washed sequentially twice in buffer A, twice in buffer A/TI mixture (buffer A, buffer TI=1:3, v/v), and once in buffer TI (25 mM tris-HCl, pH 6.8, and 20 mM imidazole). The ubiquitinated proteins were finally separated by 7% SDS-PAGE and blotted with individual antibody.

### Experimental Methods: Western Blot Assay

Cells were lysed in EBC buffer supplemented with protease inhibitors cocktail and phosphatase inhibitors, and the protein concentrations were measured as described above. The lysates (40 ug protein) were then resolved by 10% SDS-PAGE at 130 V for 80 min and immunoblotted with indicated antibodies at 4° C. overnight, washed 4 times with Tris-buffered saline with 0.1% Tween-20 (TBST), incubated with secondary antibody for 1 hour at room temperature, and then washed 4 times with TBST buffer. Anti-p65 (#8742, 1:1,000) and E2F1 (#3742, 1:500) antibodies were purchased from Cell Signaling Technologies. Anti-HA antibody (#901513, 1:1,000) was purchased from Biolegend. Anti-Flag (F3165, 1:5,000), anti-vinculin antibody (V-4505, 1:50,000), peroxidase-conjugated anti-mouse secondary antibody (A-4416, 1:3,000) and peroxidase-conjugated anti-rabbit secondary antibody (A-4914, 1:3,000) were purchased from Sigma. All primary antibodies were diluted in 5% bovine serum albumin (BSA) in TBST buffer, and secondary antibodies were diluted in 5% non-fat milk in TBST buffer.

### Experimental Methods: Purification of the GST-VHL/Elongin B/C Complex

pGEX-GST-VHL/ElonginB construct was kindly gift from Dr. William Kaelin, Jr. at Dana-Farber Cancer Institute, Harvard Medical School. pGEX-GST-Elongin C plasmid was generated by subcloning Elongin C CDS into the BamHI and Xhol sites of pGEX-4T1-GST vector. The GST-VHL/ElonginB/C complex was purified as described 43. Briefly, BL21(DE3) E. coli was cotransfected with pGEX-GST-VHL/ElonginB and pGEX-GSTElonginC plasmid. Protein expression was induced by 100 μM isopropyl beta-D-thiogalactopyranoside (IPTG, Fisher Scientific) at 18° C.overnight. Then, the bacterial culture was centrifuged and sonicated in PBS buffer with protease inhibitor cocktail. Protein lysates were further purified with glutathione-Sepharose beads (GE17-0756-05) and washed with PBS for 5 times.

### Experimental Methods: In Vivo GST Pulldown Assay

Glutathione-Sepharose beads loaded with GST-VHL E3 ligase complex were incubated at 4° C. with cell lysate derived from HEK293T cells that ectopic transfected with individual transfection factor. After incubation for 3 hours, TF-PROTACs were added into the mixture for another 2 hours, followed by wash with NETN buffer for 3 times. The pulldown products were further separated by 10% SDS-PAGE and blotted for indicated proteins.

### Experimental Methods: Proteomics Sample Preparation

HeLa cells were treated with either vehicle or dNF-κB #16 for 12 hours, washed twice with cold PBS, and then harvested in PBS to pellet down cells. Cell pellets were resuspended in lysis buffer (δ M Urea, 50 mM Tris-HCl, pH 8.0), reduced with dithiothreitol (5 mM final) for 30 min at room temperature, and alkylated with iodoacetamide (15 mM final) for 45 min in the dark at room temperature. Samples were diluted 4-fold with digestion buffer (25 mM Tris-HCl, pH 8.0, 1 mM CaCl2) and digested with trypsin at 1:100 ratio (trypsin/protein, w/w) overnight at room temperature. Peptides were desalted on home-made C₁₈ stagetips. There were two biological samples at each condition.

### Experimental Methods: Mass Spectrometry Analysis

Dried peptides were dissolved in 0.1% formic acid, 2% acetonitrile. Peptide concentration was measured with Pierce™ Quantitative Colorimetric Peptide Assay (Thermofisher). 0.5 ug of peptides were analyzed on a Q-Exactive HF-X coupled with an Easy nanoLC 1200 (Thermo Fisher Scientific, San Jose, CA). Peptides were loaded on to a nanoEase MZ HSS T3 Column (100 Å, 1.8 μm, 75 μm×150 mm, Waters). Analytical separation of all peptides was achieved with 130-min gradient. A linear gradient of 5 to 30% buffer B over 100 min, 30% to 45% buffer B over 20 min was executed at a 250 nL/min flow rate followed a ramp to 100% B in 1 min and 9-min wash with 100% B, where buffer A was aqueous 0.1% formic acid, and buffer B was 80% acetonitrile and 0.1% formic acid. LC-MS experiments were also carried out in a data-dependent mode with full MS (externally calibrated to a resolution of 60,000 at m/z 200) followed by high energy collision-activated dissociation-MS/MS of the top 15 most intense ions with a resolution of 15,000 at m/z 200. High energy collision-activated dissociation-MS/MS was used to dissociate peptides at a normalized collision energy of 27 eV in the presence of nitrogen bath gas atoms. Dynamic exclusion was 30 seconds. Each sample was subjected to two replicate technical LC-MS analyses.

### Experimental Methods: Raw Proteomics Data Processing and Analysis

Mass spectra were processed, and peptide identification was performed using the MaxQuant software version 1.6.10.43 (Max Planck Institute, Germany). Protein database searches were performed against the UniProt human protein sequence database (UP000005640). A false discovery rate (FDR) for both peptidespectrum match (PSM) and protein assignment was set at 1%. Search parameters included up to two missed cleavages at Lys/Arg on the sequence, oxidation of methionine, and protein Nterminal acetylation as a dynamic modification. Carbamidomethylation of cysteine residues was considered as a static modification. Peptide identifications were reported by filtering of reverse and contaminant entries and assigning to their leading razor protein. Data processing and statistical analysis were performed on Perseus (Version 1.6.10.50). Quantitation using iBAQ intensity (Intensity-Based Absolute Quantification) was performed with a p-value of 0.01 to report statistically significant abundance fold-changes.

### Experimental Methods: Cell proliferation and clonal formation assay

For cell growth curve assay, HeLa cells (10, 000/well) in 12-well plate were transfected with individual TF-PROTAC, then the cells were trypsined and the cell number were counted at indicated time. For the clonal formation assay, HeLa cells were transfected with individual TF-PROTAC for 12 hours, followed by further plated into 6-well plate (1,000/well). Three weeks later, the cells were fixed in fixation buffer (acetic acid: methanol=1:7) and stained with 0.4% crystal violet in 20% ethanol. Then clonal number were quantified by Image J software. As various changes can be made in the above-described subject matter without departing from the scope and spirit of the present disclsoure, it is intended that all subject matter contained in the above description, or defined in the appended claims, be interpreted as descriptive and illustrative. Many modifications and variations of the present disclosure are possible in light of the above teachings. Accordingly, the present description is intended to embrace all such alternatives, modifications and variances which fall within the scope of the appended claims.

1. A compound having the structure of formula (I):
ODN—L₁—ULB (I)
wherein ULB is a ubiquitin ligase binding moiety;
L₁ is absent or a linker comprising an azide/alkyne cycloaddition reaction product;
ODN is a DNA oligomer protein comprising a transcription factor binding motif that binds to a transcription factor or subunit thereof;
or pharmaceutically acceptable salts thereof.

2. The compound according to claim 1, wherein L₁ comprises a strained click chemistry reaction product.

3. The compound according to claim 1, wherein L₁ has the structure of formula (IV):
—Y₁—Y₂—Y₃—Y₄—Y₅—Y₆—Y₇—Y₈— (IV)
wherein Y₁—Y₈ are independently selected from absent, a heteroarylene group, a heterocyclene group, —C(O)—, —O—, —OC(O)—, —NR^{a}—, —N(R^{a})C(O)—, —(C(R^{a})(R^{a}))₁₋₁₂, —(C(R^{a})(R^{a})C(R^{a})(R^{a})O)₁₋₁₂—, and —S—S—; and
R^{a} is independently selected at each occurrence from hydrogen and optionally substitued alkyl.

4. The compound according to claim 1, wherein said compound has the structure of formula (IVa), (IVb), (IVc), (IVd), (IVf), (IVg), (IVh), (IVi), (IVj), (IVk), (IVl), (IVm), (IVn), (IVo), (IVp), (IVq), (IVr):
ODN—Y₁—Y₂—Y₃—NH—(CH₂)₁₋₁₂—NH—C(O)—ULB (IVa)
ODN—Y₁—Y₂—Y₃—(CH₂)₁₋₁₂—NH—C(O)—(CH₂)₁₋₁₂—ULB (IVb)
ODN—Y₁—Y₂—Y₃—(CH₂)₁₋₁₂—NH—(CH₂)₁₋₁₂—ULB (IVc)
ODN—Y₁—Y₂—Y₃—NH—(CH₂)₁₋₁₂—NH—C(O)—ULB (IVd)
ODN—Y₁—Y₂—Y₃—C(O)—(CH₂)₁₋₁₂—ULB (IVf)
ODN—Y₁—Y₂—Y₃—NH—(CH₂)₁₋₁₂—ULB (IVg)
ODN—Y₁—Y₂—Y₃—NH—(CH₂CH₂O)₁₋₁₂—NH—C(O)—ULB (IVh)
ODN—Y₁—Y₂—Y₃—(CH₂CH₂O)₁₋₁₂—NH—C(O)—(CH₂CH₂O)₁₋₁₂—ULB (IVi)
ODN—Y₁—Y₂—Y₃—(CH₂CH₂O)₁₋₁₂—NH—(CH₂CH₂O)₁₋₁₂—ULB (IVj)
ODN—Y₁—Y₂—Y₃—NH—(CH₂CH₂O)₁₋₁₂—NH—C(O)—ULB (IVk)
ODN—Y₁—Y₂—Y₃—C(O)—(CH₂CH₂O)₁₋₁₂—ULB (IVl)
ODN—Y₁—Y₂—Y₃—NH—(CH₂CH₂O)₁₋₁₂—ULB (IVm)
ODN—Y₁—Y₂—Y₃—(CH₂)₁₋₁₂—C(O)—NH—(CH₂)₁₋₁₂—ULB (IVn)
ODN—Y₁—Y₂—Y₃—(CH₂)₁₋₁₂—OC(O)—NH—(CH₂)₁₋₁₂—ULB (IVo)
ODN—Y₁—Y₂—Y₃—(CH₂)₁₋₁₂—OC(O)—NH—(CH₂CH₂O)₁₋₁₂—ULB (IVp)
ODN—Y₁—Y₂—Y₃—(CH₂)₁₋₁₂—OC(O)—NH—(CH₂)₁₋₁₂—(CH₂CH₂O)₁₋₁₂—ULB (IVq)
ODN—Y₁—Y₂—Y₃—(CH₂)₁₋₁₂—OC(O)—NH—(CH₂CH₂O)₁₋₁₂—(CH₂)₁₋₁₂—ULB (IVr)
wherein Y₁—Y₃ are independently selected from absent, a multicyclic heteroarylene group, a multicyclic heterocyclene group, —C(O)—, —O—, —OC(O)—, —NR^{a}—, —N(R^{a})C(O)—, (C(R^{a})(R^{a}))₁₋₁₂, —(C(R^{a})(R^{a})C(R^{a})(R^{a})O)₁₋₁₂—, and —S—S—; and
R^{a} is independently selected at each occurrence from hydrogen and alkyl.

5. The compound according to claim 3, wherein Y₂ or Y₃ is a strained click chemistry reaction product having the structure: 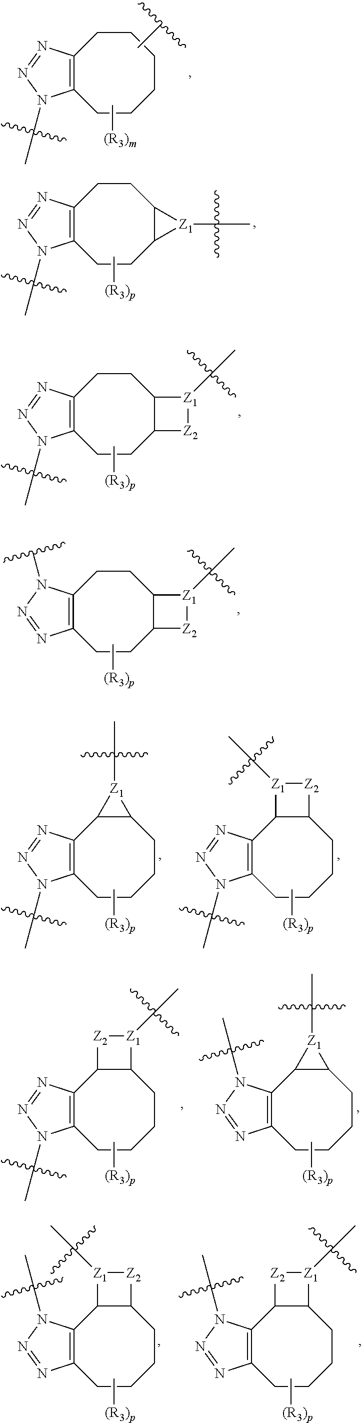 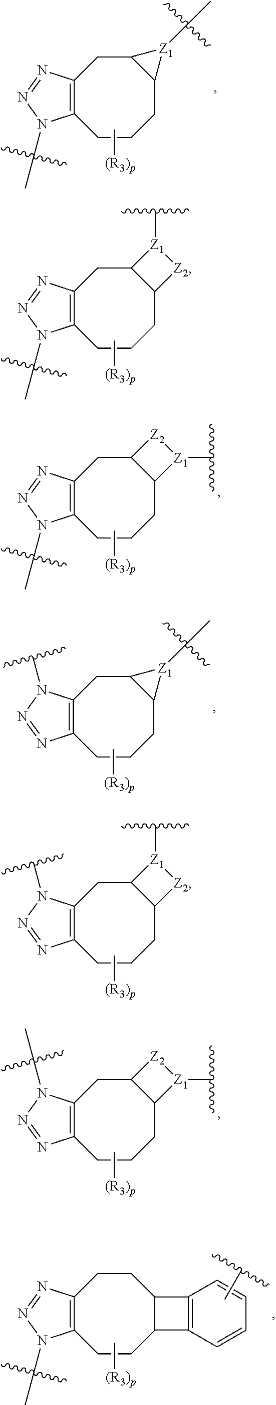 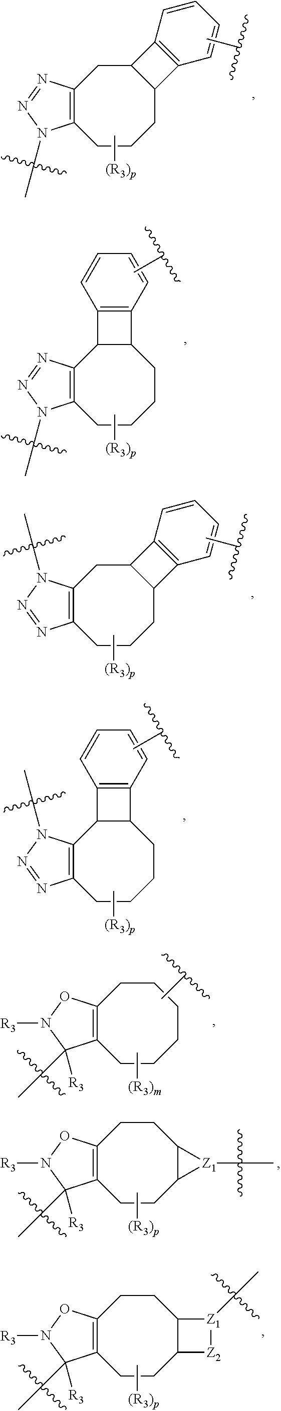 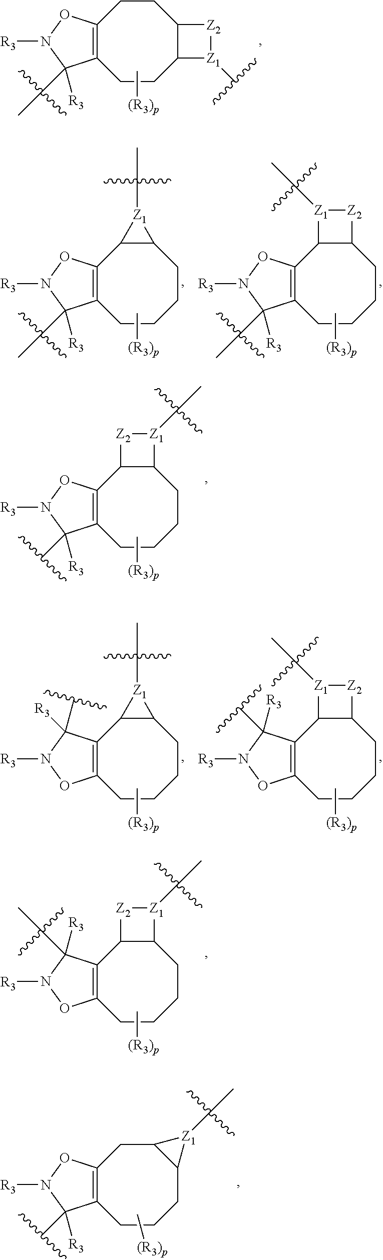 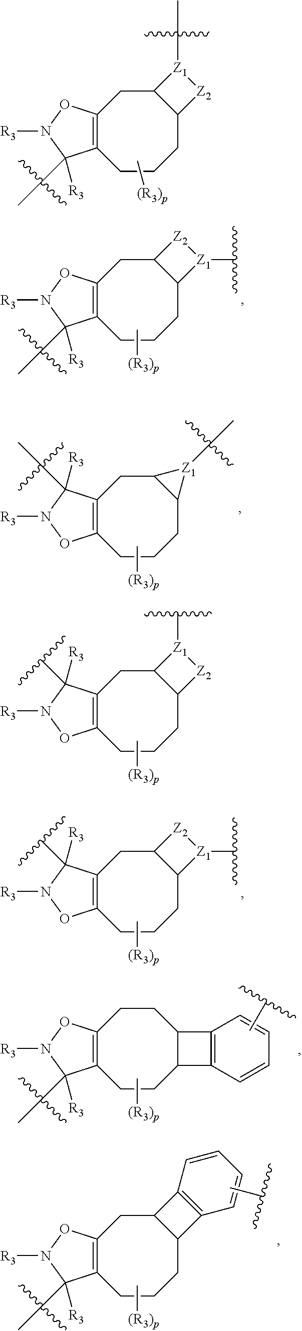 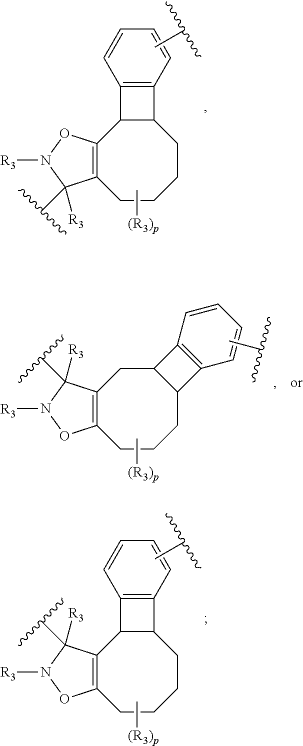
wherein each 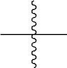
indicates the point of attachment to the neighboring linker group;
m is an integer from 0-12 (0, 1, 2, 3, 4, 5, 6, 7, 8, 9, 10, 11, or 12);
p is an integer from 0-10 (0, 1, 2, 3, 4, 5, 6, 7, 8, 9, or 10);
Z₁ is —N— or —CR₃—;
Z₂ is O, C(O), or C(R₃)₂; and
R₃ is independently selected at each occurrence from hydrogen, —N(R^{a})(R^{a}), alkyl, or alkoxy and wherein any two vicinal R₃ groups of the C₈ ring may together form a five or six membered optionally aromatic ring fused to the C₈ ring.

6. The compound according to claim 3, wherein at least one of Y₁-Y₇ has the structure: 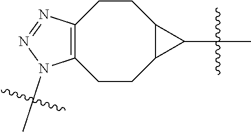 [SMILES: *C1C2CCc3nnn(*)c3CCC12]
wherein each 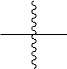
indicates the point of attachment to the neighboring linker group.

7. The compound according to claim 3, wherein said compound has the structure of formula (V):

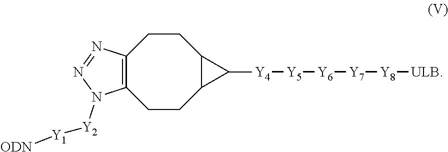

8. The compound according to claim 3, wherein Y₈ is absent and Y₇ is —(CH₂)₁₋₁₂—.

9. The compound according to claim 3, wherein Y₇ is —(CH₂C H₂O)₁₋₁₂— and Y₈ is —(CH₂)₁₋₁₂—.

10. The compound according to claim 1, wherein ODN has a double-band hairpin structure.

11. The compound according to claim 1, wherein ODN is a double-stranded DNA comprising a sense chain and an anti-sense chain.
wherein R is purine, Y is pyrimidine, and N is any base.

12. The compound according to claim 1, wherein ODN comprises the sequence
(SEQ ID NO: 2)
ACGGACCGGAAATCCGGTT,
(SEQ ID NO: 3)
TACAAAGATCAAAGGGTT,
ATCAAA,
(SEQ ID NO: 4)
TGGGGACTTTCCAGTTTCTGGAAAGTCCCCA,
(SEQ ID NO: 5)
CTAGATTTCCCGCG,
or
(SEQ ID NO: 6)
CTAGCGCGGGAAAT.

13. The compound according to claim 1, wherein the ODN oligonucleotide is conjugated to L₁ through the 5′ end of the oligonucleotide.

14. The compound according to claim 1, wherein said compound is Compound 1 (dNF-κB #1), Compound 2 (dNF-κB #2), Compound 3 (dNF-κB #3), Compound 4 (dNF-κB #4), Compound 5 (dNF-κB #5), Compound 6 (dNF-κB #6), Compound 7 (dNF-κB #7), Compound δ (dNF-κB #8), Compound 9 (dNF-κB #9), Compound 10 (dNF-κB #10), Compound 11 (dNF-κB #11), Compound 12 (dNF-κB #12), Compound 13 (dNF-κB #13), Compound 14 (dNF-κB #14), Compound 15 (dNF-κB #15), Compound 16 (dNF-κB #16), Compound 17 (dNF-κB #17), Compound 18 (dNF-κB #18), Compound 19 (dE2F #1), Compound 20 (dE2F #2), Compound 21 (dE2F #3), Compound 22 (dE2F #4), Compound 23 (dE2F #5), Compound 24 (dE2F #6), Compound 25 (dE2F #7), Compound 26 (dE2F #8), Compound 27 (dE2F #9), Compound 28 (dE2F #10), Compound 29 (dE2F #11), Compound 30 (dE2F #12), Compound 31 (dE2F #13), Compound 32 (dE2F #14), Compound 33 (dE2F #15), Compound 34 (dE2F #16), Compound 35 (dE2F #17), or Compound 36 (dE2F #18).

15. A pharmaceutical composition comprising the compound according to claim 1 and one or more pharmaceutically acceptable salts, carriers, or diluents.

16. The pharmaceutical composition according to claim 15 wherein said compound is formulated in a liposome.

17. A method for reducing the proliferation or survival of a neoplastic cell or a virus, the method comprising contacting the cell or virus with a compound according to claim 1 to induce degradation of transciption factors or subunits thereof in the cell or virus.

18. A method for the treatment or prophylaxis of a proliferative disease or virus in a subject in need thereof comprising administering a compound according to claim 1.

19. A compound having the structure of formula (VI):
Y₂—Y₃—Y₄—Y₅—Y₆—Y₇—Y₈—ULB (VI)
wherein ULB is a ubiquitin ligase binding moiety; and
wherein Y₂ is alkynyl, monocyclic or bicyclic cycloalkynyl, heteroalkynyl, or monocyclic or bicyclic heterocycloalkynyl;
Y₃-Y₈ are independently selected from absent, a heteroarylene group, a heterocyclene group, —C(O)—, —O—, —OC(O)—, —NR^{a}—, —N(R^{a})C(O)—, —(C(R^{a})(R^{a}))₁₋₁₂, —(C(R^{a})(R^{a})C(R^{a})(R^{a})O)₁₋₁₂—, and —S—S—; and
R^{a} is independently selected at each occurrence from hydrogen and optionally substitued alkyl.

20. A method of forming the compound according to claim 1 comprising reacting a DNA oligomer protein comprising a transcription factor binding motif that binds to a transcription factor or subunit thereof; where said DNA oligomer protein is conjugated to an azide
with a compound having the structure of formula (VI):
Y₂—Y₃—Y₄—Y₅—Y₆—Y₇—Y₈—ULB (VI)
wherein ULB is a ubiquitin ligase binding moiety; and
wherein Y₂ is alkynyl, monocyclic or bicyclic cycloalkynyl, heteroalkynyl, or monocyclic or bicyclic heterocycloalkynyl;
Y₃-Ys are independently selected from absent, —C(O)—, —O—, —OC(O)—, —NR₂—, —N(R^{a})C(O)—, —C(R^{a})(R^{a}))₁₋₁₂, —(C(R^{a})(R^{a})C(R^{a})(R^{a})O)₁₋₁₂—, and —S—S—; and
R^{a} is independently selected at each occurrence from hydrogen and optionally substitued alkyl.
